(19) United States
(12) Patent Application Publication
(10) Pub. No.: US 2010/0099101 A1
(43) Pub. Date: Apr. 22, 2010

(54) METHODS FOR TREATING, DIAGNOSING, AND MONITORING LUPUS

(75) Inventors: Timothy W. Behrens, Burlingame, CA (US); Robert R. Graham, San Francisco, CA (US); Geoffrey Hom, Seattle, WA (US); Ward A. Ortmann, Walnut Creek, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 12/567,669

(22) Filed: Sep. 25, 2009

Related U.S. Application Data
(60) Provisional application No. 61/100,659, filed on Sep. 26, 2008.

(51) Int. Cl. C12Q 1/68

(52) U.S. Cl. 435/6

(57) ABSTRACT

Methods of identifying, diagnosing, and prognosing lupus, including certain subphenotypes of lupus, are provided, as well as methods of treating lupus, including certain subpopulations of patients. Also provided are methods for identifying effective lupus therapeutic agents and predicting responsiveness to lupus therapeutic agents.

### CROSS REFERENCE TO RELATED APPLICATION

This application claims the benefit of priority of provisional U.S. Application No. 61/100,659 filed Sep. 26, 2008, which is hereby incorporated by reference in its entirety.

### FIELD

Methods of identifying, diagnosing, and prognosing lupus, including certain subphenotypes of lupus, are provided, as well as methods of treating lupus, including certain subpopulations of patients. Also provided are methods for identifying effective lupus therapeutic agents and predicting responsiveness to lupus therapeutic agents.

### BACKGROUND

Lupus is an autoimmune disease that is estimated to affect nearly 1 million Americans, primarily women between the ages of 20-40. Lupus involves antibodies that attack connective tissue. The principal form of lupus is a systemic one (systemic lupus erythematosus; SLE). SLE is a chronic autoimmune disease with strong genetic as well as environmental components (See, e.g., Hochberg M C, Dubois' Lupus Erythematosus. 5th ed., Wallace D J, Hahn B H, eds. Baltimore: Williams and Wilkins (1997); Wakeland E K, et al., Immunity 2001; 15(3):397-408; Nath S K, et al., Curr. Opin. Immunol. 2004; 16(6):794-800; D'Cruz et al., Lancet (2007), 369:587-596). Various additional forms of lupus are known, including, but not limited to, cutaneous lupus erythematosus (CLE), lupus nephritis (LN), and neonatal lupus.

Untreated lupus can be fatal as it progresses from attack of skin and joints to internal organs, including lung, heart, and kidneys (with renal disease being the primary concern), thus making early and accurate diagnosis of and/or assessment of risk of developing lupus particularly critical. Lupus mainly appears as a series of flare-ups, with intervening periods of little or no disease manifestation. Kidney damage, measured by the amount of proteinuria in the urine, is one of the most acute areas of damage associated with pathogenicity in SLE, and accounts for at least 50% of the mortality and morbidity of the disease.

Clinically, SLE is a heterogeneous disorder characterized by high-affinity autoantibodies (autoAbs). AutoAbs play an important role in the pathogenesis of SLE, and the diverse clinical manifestations of the disease are due to the deposition of antibody-containing immune complexes in blood vessels leading to inflammation in the kidney, brain and skin. AutoAbs also have direct pathogenic effects contributing to hemolytic anemia and thrombocytopenia. SLE is associated with the production of antinuclear antibodies, circulating immune complexes, and activation of the complement system. SLE has an incidence of about 1 in 700 women between the ages of 20 and 60. SLE can affect any organ system and can cause severe tissue damage. Numerous autoAbs of differing specificity are present in SLE. SLE patients often produce autoAbs having anti-DNA, anti-Ro, and anti-platelet specificity and that are capable of initiating clinical features of the disease, such as glomerulonephritis, arthritis, serositis, complete heart block in newborns, and hematologic abnormalities. These autoAbs are also possibly related to central nervous system disturbances. Arbuckle et al. described the development of autoAbs before the clinical onset of SLE (Arbuckle et al. N. Engl. J. Med. 349(16): 1526-1533 (2003)).

AutoAbs recognizing RNA-binding proteins (RBPs; also referred to as extractable nuclear antigens) were first characterized in SLE over 40 years ago (Holman, Ann N Y Acad. Sci. 124(2):800-6 (1965)). Such RBPs comprise a group of proteins—SSA (Ro52/TRIM21 and Ro60/TROVE2), SSB (La), ribonucleoprotein (RNP/U1 small nuclear RNP complex) and Smith autoantigen complex (Sm)—with roles in RNA processing and biochemistry. Anti-SSA—and anti-SSB IgG autoAbs are found not only in SLE, but also rheumatoid arthritis and Sjôgren's syndrome. Anti-SSA autoAbs are associated with subacute cutaneous lupus erythematosus, and with congenital heart block and neonatal lupus in children of anti-SSA positive women. Anti-SSB autoAbs are nearly always found together with anti-SSA autoAbs, and both autoantigens associate with cytoplasmic hYRNA (Lerner et al., Science 211(4480):400-2 (1981)). Anti-Sm autoAbs are highly specific for SLE and are generally found together with anti-RNP autoAbs. Both Sm and RNP proteins associate with common snRNA species in the nuclear RNA spliceosome (Lerner et al., Proc Natl Acad Sci USA 76(11):5495-9 (1979)). Anti-RNP autoAbs are also found in patients with mixed connective tissue disease. It has been suggested that the presence of anti-RBP autoAbs may identify SLE cases that show less durable responses following B cell depletion therapy (Cambridge et al., Ann Rheum Dis 67:1011-16 (2008))

Recent reports show, in certain instances, that the type I interferon (IFN) pathway plays an important role in SLE disease pathogenesis. Type I IFN is present in serum of SLE cases, and production of IFN is linked to the presence of Ab and nucleic acid containing immune complexes (reviewed in Ronnblom et al., J Exp Med 194:F59 (2001)). The majority of SLE cases exhibit a prominent type I IFN gene expression ‘signature’ in blood cells (Baechler et al., Proc Natl Acad Sci USA 100:2610 (2003); Bennett et al., J Exp Med 197:711 (2003)) and have elevated levels of IFN-inducible cytokines and chemokines in serum (Bauer et al., PLoS Med 3:e491 (2006)). Immune complexes containing native DNA and RNA stimulate toll-like receptors (TLRs) 7 and 9 expressed by dendritic cells and B cells to produce type I interferon which further stimulates immune complex formation (reviewed in (Marshak-Rothstein et al., Annu Rev Immunol 25, 419 (2007)).

One of the most difficult challenges in clinical management of complex autoimmune diseases such as lupus is the accurate and early identification of the disease in a patient. In addition, no reliable diagnostic markers, e.g., biomarkers, have been identified that enable clinicians or others to accurately define pathophysiological aspects of SLE, clinical activity, response to therapy, or prognosis, although a number of candidate genes and alleles (variants) have been identified that are thought to contribute to SLE susceptibility. For example, at least 13 common alleles that contribute risk for SLE in individuals of European ancestry have been reported (Kyogoku et al., Am J Hum Genet. 75(3):504-7 (2004); Sigurdsson et al., Am J Hum Genet. 76(3):528-37 (2005); Graham et al., Nat Genet. 38(5):550-55 (2006); Graham et al., Proc Natl Acad Sci USA 104(16):6758-63 (2007); Remmers et al., N Engl J Med 357(10):977-86 (2007); Cunninghame Graham et al., Nat Genet. 40(1):83-89 (2008); Harley et al., Nat Genet. 40(2):204-10 (2008); Hom et al., N Engl J Med 358(9):900-9 (2008); Kozyrev et al., Nat Genet. 40(2):211-6 (2008); Nath et al., Nat Genet. 40(2):152-4 (2008); Sawalha et al., PLoS ONE 3(3):e1727 (2008)). The putative causal alleles are known for HLA-DR3, HLA-DR2, FCGR2A, PTPN22, ITGAM and BANK1 (Kyogoku et al., Am J Hum Genet. 75(3):504-7 (2004); Kozyrev et al., Nat Genet. 40(2):211-6 (2008); Nath et al., Nat Genet. 40(2):152-4 (2008)), while the risk haplotypes for IRF5, TNFSF4 and BLK likely contribute to SLE by influencing mRNA and protein expression levels (Sigurdsson et al., Am J Hum Genet. 76(3):528-37 (2005); Graham et al., Nat Genet. 38(5):550-55 (2006); Graham et al., Proc Natl Acad Sci USA 104(16):6758-63 (2007); Cunninghame Graham et al., Nat Genet. 40(1):83-89 (2008); Horn et al., N Engl J Med 358(9):900-9 (2008)). The causal alleles for STAT4, KIAA1542, IRAK1 and PXK have not been determined (Remmers et al., N Engl J Med 357(10):977-86 (2007); Harley et al., Nat Genet 40(2):204-10 (2008); Hom et al., N Engl J Med 358(9):900-9 (2008); Sawalha et al., PLoS ONE 3(3):e1727 (2008)). The contribution of such genetic variation to the significant clinical heterogeneity of SLE remains unknown.

It would therefore be highly advantageous to have molecular-based diagnostic methods that can be used to objectively identify the presence of and/or classify the disease in a patient, define pathophysiologic aspects of lupus, clinical activity, response to therapy, or prognosis. In addition, it would be advantageous to have molecular-based diagnostic markers associated with various clinical and/or pathophysiological and/or other biological indicators of disease, such as, but not limited to, the presence or absence of autoAbs. Such associations would greatly benefit the identification of the presence of lupus in patients or the determination of susceptibility to develop the disease. Such associations would also benefit the identification of pathophysiologic aspects of lupus, clinical activity, response to therapy, or prognosis. In addition, statistically and biologically significant and reproducible information regarding such associations could be utilized as an integral component in efforts to identify specific subsets of patients who would be expected to significantly benefit from treatment with a particular therapeutic agent, for example where the therapeutic agent is or has been shown in clinical studies to be of therapeutic benefit in such specific lupus patient subpopulation.

The invention described herein meets the above-described needs and provides other benefits.

All references cited herein, including patent applications and publications, are incorporated by reference in their entirety for any purpose.

### SUMMARY

The methods of the invention are based, at least in part, on the discovery of a set of loci that are associated with SLE and that contribute disease risk (SLE risk loci). In addition, the invention includes a set of alleles associated with the SLE risk loci. A further aspect of the invention is the discovery of the association of certain SLE risk loci with a subphenotype of SLE involving autoantibodies to RNA binding proteins, induction of expression of genes in the type I interferon pathway and/or early onset of disease.

In one aspect, a method of identifying lupus in a subject is provided, the method comprising detecting in a biological sample derived from the subject the presence of a variation in each of at least three SLE risk loci as set forth in Table 2, wherein the variation at each locus occurs at a nucleotide position corresponding to the position of a single nucleotide polymorphism (SNP) for each of the loci as set forth in Table 2, and wherein the subject is suspected of suffering from lupus. In certain embodiments, a variation is detected in at least four loci, or at least five loci, or at least seven loci, or at least ten loci, or at least 12 loci. In one embodiment, a variation is detected in 16 loci. In one embodiment, the three SLE risk loci are PTTG1, ATG5, and UBE2L3. In one embodiment, the variation at each locus is a genetic variation. In one embodiment, each variation comprises a SNP as set forth in Table 2. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In another aspect, a method for predicting responsiveness of a subject with lupus to a lupus therapeutic agent is provided, the method comprising determining whether the subject comprises a variation in each of at least three SLE risk loci as set forth in Table 2, wherein the variation at each locus occurs at a nucleotide position corresponding to the position of a single nucleotide polymorphism (SNP) for each of the loci as set forth in Table 2, wherein the presence of a variation at each locus indicates the responsiveness of the subject to the therapeutic agent. In certain embodiments, the subject comprises a variation in at least four loci, or at least five loci, or at least seven loci, or at least ten loci, or at least 12 loci. In one embodiment, the subject comprises a variation in 16 loci. In one embodiment, the three SLE risk loci are PTTG1, ATG5, and UBE2L3. In one embodiment, the variation at each locus is a genetic variation. In one embodiment, each variation comprises a SNP as set forth in Table 2.

In yet another aspect, a method of diagnosing or prognosing lupus in a subject is provided, the method comprising detecting in a biological sample derived from the subject the presence of a variation in each of at least three SLE risk loci as set forth in Table 2, wherein: the biological sample is known to comprise, or suspected of comprising, nucleic acid comprising at least three SLE risk loci as set forth in Table 2, each locus comprising a variation; the variation at each locus comprises, or is located at a nucleotide position corresponding to, a SNP as set forth in Table 2; and the presence of the variation at each locus is a diagnosis or prognosis of lupus in the subject. In certain embodiments, a variation is detected in at least four loci, or at least five loci, or at least seven loci, or at least ten loci, or at least 12 loci. In one embodiment, a variation is detected in 16 loci. In one embodiment, the three SLE risk loci are PTTG1, ATG5, and UBE2L3.

In a still further aspect, a method of aiding in the diagnosis or prognosis of lupus in a subject is provided, the method comprising detecting in a biological sample derived from the subject the presence of a variation in each of at least three SLE risk loci as set forth in Table 2, wherein: the biological sample is known to comprise, or suspected of comprising, nucleic acid comprising at least three SLE risk loci as set forth in Table 2, each locus comprising a variation; the variation at each locus comprises, or is located at a nucleotide position corresponding to, a SNP as set forth in Table 2; and the presence of the variation at each locus is a diagnosis or prognosis of lupus in the subject. In certain embodiments, a variation is detected in at least four loci, or at least five loci, or at least seven loci, or at least ten loci, or at least 12 loci. In one embodiment, a variation is detected in 16 loci. In one embodiment, the three SLE risk loci are PTTG1, ATG5, and UBE2L3.

In one aspect, a method of treating a lupus condition in a subject in whom a genetic variation is known to be present at a nucleotide position corresponding to a single nucleotide polymorphism (SNP) as set forth in Table 2 in each of at least three SLE risk loci as set forth in Table 2 is provided, the method comprising administering to the subject a therapeutic agent effective to treat the condition. In one embodiment, the three SLE risk loci are PTTG1, ATG5, and UBE2L3.

In another aspect, a method of treating a subject having a lupus condition is provided, the method comprising administering to the subject a therapeutic agent effective to treat the condition in a subject who has a genetic variation at a nucleotide position corresponding to a single nucleotide polymorphism (SNP) as set forth in Table 2 in each of at least three SLE risk loci as set forth in Table 2. In one embodiment, the three SLE risk loci are PTTG1, ATG5, and UBE2L3.

In yet another aspect, a method of treating a subject having a lupus condition is provided, the method comprising administering to the subject a therapeutic agent shown to be effective to treat said condition in at least one clinical study wherein the agent was administered to at least five human subjects who each had a genetic variation at a nucleotide position corresponding to a single nucleotide polymorphism (SNP) as set forth in Table 2 in each of at least three SLE risk loci as set forth in Table 2. In one embodiment, the three SLE risk loci are PTTG1, ATG5, and UBE2L3.

In one aspect, a method of identifying a subphenotype of lupus in a subject is provided, the method comprising detecting in a biological sample derived from the subject the presence of a variation in each of at least three SLE risk loci selected from HLA-DR3, HLA-DR2, TNFSF4, IRAK1, STAT4, UBE2L3, and IRF5, wherein the variation at each locus occurs at a nucleotide position corresponding to the position of a single nucleotide polymorphism (SNP) for each of the loci as set forth in Table 2, and wherein the subject is suspected of suffering from lupus and is suspected of having a subphenotype of lupus. In certain embodiments, a variation is detected in at least four loci or at least five loci. In one embodiment, a variation is detected in 7 loci. In one embodiment, the variation at each locus is a genetic variation. In one embodiment, each variation comprises a SNP as set forth in Table 2. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP and Sm. In one embodiment, the biological sample is serum. In one embodiment, the subphenotype of lupus is characterized at least in part by higher levels of interferon inducible gene expression in a biological sample derived from the subject as compared to one or more control subjects. In one embodiment, the subphenotype of lupus is characterized at least in part by the presence of autoantibodies in a biological sample derived from the subject to one or more RNA binding proteins and by higher levels of interferon inducible gene expression in a biological sample derived from the subject as compared to one or more control subjects.

In another aspect, a method for predicting responsiveness of a subject with an identified lupus subphenotype to a lupus therapeutic agent is provided, the method comprising determining whether the subject comprises a variation in each of at least three SLE risk loci selected from HLA-DR3, HLA-DR2, TNFSF4, IRAK1, STAT4, UBE2L3, and IRF5, wherein the variation at each locus occurs at a nucleotide position corresponding to the position of a single nucleotide polymorphism (SNP) for each of the loci as set forth in Table 2, wherein the presence of a variation at each locus indicates the responsiveness of the subject to the therapeutic agent. In certain embodiments, the subject comprises a variation in at least four loci or at least five loci. In one embodiment, the subject comprises a variation in 7 loci. In one embodiment, the variation at each locus is a genetic variation. In one embodiment, each variation comprises a SNP as set forth in Table 2.

In yet another aspect, a method of diagnosing or prognosing a subphenotype of lupus in a subject, the method comprising detecting in a biological sample derived from the subject the presence of a variation in each of at least three SLE risk loci, wherein: the biological sample is known to comprise, or suspected of comprising, nucleic acid comprising at least three SLE risk loci selected from HLA-DR3, HLA-DR2, TNFSF4, IRAK1, STAT4, UBE2L3, and IRF5, each locus comprising a variation; the variation at each locus comprises, or is located at a nucleotide position corresponding to, a SNP as set forth in Table 2; and the presence of the variation at each locus is a diagnosis or prognosis of the subphenotype of lupus in the subject. In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP, and Sm. In one embodiment, the biological sample is serum. In one embodiment, the subphenotype of lupus is characterized at least in part by higher levels of interferon inducible gene expression in a biological sample derived from the subject as compared to one or more control subjects. In one embodiment, the subphenotype of lupus is characterized at least in part by the presence of autoantibodies in a biological sample derived from the subject to one or more RNA binding proteins and by higher levels of interferon inducible gene expression in a biological sample derived from the subject as compared to one or more control subjects.

In a still further aspect, a method of aiding in the diagnosis or prognosis of lupus in a subject, the method comprising detecting in a biological sample derived from the subject the presence of a variation in each of at least three SLE risk loci, wherein: the biological sample is known to comprise, or suspected of comprising, nucleic acid comprising at least three SLE risk loci selected from HLA-DR3, HLA-DR2, TNFSF4, IRAK1, STAT4, UBE2L3, and IRF5, each locus comprising a variation; the variation at each locus comprises, or is located at a nucleotide position corresponding to, a SNP as set forth in Table 2; and the presence of the variation at each locus is a diagnosis or prognosis of the subphenotype of lupus in the subject. In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP, and Sm. In one embodiment, the biological sample is serum. In one embodiment, the subphenotype of lupus is characterized at least in part by higher levels of interferon inducible gene expression in a biological sample derived from the subject as compared to one or more control subjects. In one embodiment, the subphenotype of lupus is characterized at least in part by higher levels of interferon inducible gene expression in a biological sample derived from the subject as compared to one or more control subjects. In one embodiment, the subphenotype of lupus is characterized at least in part by the presence of autoantibodies in a biological sample derived from the subject to one or more RNA binding proteins and by higher levels of interferon inducible gene expression in a biological sample derived from the subject as compared to one or more control subjects.

In one aspect, a method of treating a lupus condition in a subject in whom a genetic variation is known to be present at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5 is provided, wherein the lupus condition is characterized at least in part by the presence of autoantibodies in a biological sample derived from the subject to one or more RNA binding proteins and/or by higher levels of interferon inducible gene expression in a biological sample derived from the subject as compared to one or more control subjects, the method comprising administering to the subject a therapeutic agent effective to treat the condition.

In another aspect, a method of treating a subject having a lupus condition is provided, the method comprising administering to the subject a therapeutic agent effective to treat the condition in a subject who has a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5, wherein the lupus condition is characterized at least in part by the presence of autoantibodies in a biological sample derived from the subject to one or more RNA binding proteins and/or by higher levels of interferon inducible gene expression in a biological sample derived from the subject as compared to one or more control subjects.

In yet another aspect, a method of treating a subject having a lupus condition is provided, the method comprising administering to the subject a therapeutic agent shown to be effective to treat said condition in at least one clinical study wherein the agent was administered to at least five human subjects who each had a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5, wherein the lupus condition is characterized at least in part by the presence of autoantibodies in a biological sample derived from the subject to one or more RNA binding proteins and/or by higher levels of interferon inducible gene expression in a biological sample derived from the subject as compared to one or more control subjects.

In a still further aspect, a method of identifying a therapeutic agent effective to treat lupus in a patient subpopulation, the method comprising correlating efficacy of the agent with the presence of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5 in the patient subpopulation thereby identifying the agent as effective to treat lupus in said patient subpopulation. In one embodiment, the efficacy of the agent is correlated with the presence of a genetic variation at a nucleotide position corresponding to a SNP as set forth in Table 2 in each of at least four loci, or at least five loci, or in seven loci.

In one aspect, a method of treating a lupus subject of a specific lupus patient subpopulation is provided, wherein the subpopulation is characterized at least in part by association with genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5, and wherein the method comprises administering to the subject an effective amount of a therapeutic agent that is approved as a therapeutic agent for said subpopulation. In one embodiment, the subpopulation is characterized at least in part by the presence of autoantibodies to one or more RNA binding proteins, wherein the autoantibodies are capable of being detected in a biological sample. In one embodiment, the RNA binding protein is selected from SSA, SSB, RNP and Sm. In one embodiment, the subpopulation is characterized at least in part by higher levels of interferon inducible gene expression as compared to one or more control subjects, wherein the interferon inducible gene expression is capable of being detected in a biological sample and quantified. In one embodiment, the subpopulation is female. In one embodiment, the subpopulation is of European ancestry.

In another aspect, a method comprising manufacturing a lupus therapeutic agent is provided, which includes packaging the agent with instructions to administer the agent to a subject who has or is believed to have lupus and who has a genetic variation at a position corresponding to a single nucleotide polymorphism (SNP) as set forth in Table 2 in each of at least three SLE risk loci as set forth in Table 2.

In a further aspect, a method of specifying a therapeutic agent for use in a lupus patient subpopulation is provided, the method comprising providing instructions to administer the therapeutic agent to a patient subpopulation characterized at least in part by a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5.

In a still further aspect, a method for marketing a therapeutic agent for use in a lupus patient subpopulation is provided, the method comprising informing a target audience about the use of the therapeutic agent for treating the patient subpopulation as characterized at least in part by the presence, in patients of such subpopulation, of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5.

In yet a further aspect, a method for modulating signaling through the type I interferon pathway in a subject in whom a genetic variation is known to be present at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5 is provided, the method comprising administering to the subject a therapeutic agent effective to modulate gene expression of one or more interferon inducible genes.

In one aspect, a method for selecting a patient suffering from lupus for treatment with a lupus therapeutic agent is provided, the method comprising detecting the presence of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5. In certain embodiments, a variation is detected in at least four loci or at least five loci. In one embodiment, a variation is detected in 7 loci. In one embodiment, the variation at each locus is a genetic variation. In one embodiment, each variation comprises a SNP as set forth Table 2. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay. In one embodiment, the lupus is a subphenotype of lupus characterized at least in part by the presence of autoantibodies in a biological sample derived from the patient to one or more RNA binding proteins for treatment and/or by a higher level of interferon inducible gene expression as compared to one or more control subjects. In one embodiment, the RNA binding protein is selected from SSA, SSB, RNP, and Sm.

In another aspect, a method of assessing whether a subject is at risk of developing lupus is provided, the method comprising detecting in a biological sample obtained from the subject, the presence of a genetic signature indicative of risk of developing lupus, wherein said genetic signature comprises a set of at least three single nucleotide polymorphisms (SNPs), each SNP occurring in a SLE risk locus as set forth in Table 2. In certain embodiments, the genetic signature comprises a set of at least four SNPs, or at least five SNPs, or at least seven SNPs, or at least ten SNPs, or at least 12 SNPs. In one embodiment, the genetic signature comprises a set of 16 SNPs. In one embodiment, the SLE risk loci are selected from HLA-DR3, HLA-DR2, TNFSF4, IRAK1, STAT4, UBE2L3, and IRF5. In one embodiment, the SLE risk loci are PTTG1, ATG5, and UBE2L3.

In a further aspect, a method of diagnosing lupus in a subject is provided, the method comprising detecting in a biological sample obtained from said subject, the presence of a genetic signature indicative of lupus, wherein said genetic signature comprises a set of at least three single nucleotide polymorphisms (SNPs), each SNP occurring in a SLE risk locus as set forth in Table 2. In certain embodiments, the genetic signature comprises a set of at least four SNPs, or at least five SNPs, or at least seven SNPs, or at least ten SNPs, or at least 12 SNPs. In one embodiment, the genetic signature comprises a set of 16 SNPs. In one embodiment, the SLE risk loci are selected from HLA-DR3, HLA-DR2, TNFSF4, IRAK1, STAT4, UBE2L3, and IRF5. In one embodiment, the SLE risk loci are PTTG1, ATG5, and UBE2L3.

In yet a further aspect, a method of assessing whether a subject is at risk of developing lupus characterized by the presence of autoantibodies to one or more RNA binding proteins is provided, the method comprising detecting in a biological sample obtained from the subject, the presence of a genetic signature indicative of the risk, wherein said genetic signature comprises a set of at least three single nucleotide polymorphisms (SNPs), each SNP occurring in a SLE risk locus, wherein each SLE risk locus is selected from HLA-DR3, HLA-DR2, TNFSF4, IRAK1, STAT4, UBE2L3, and IRF5. In one embodiment, the RNA binding proteins are selected from SSA, SSB, RNP and Sm.

In another aspect, a method of assessing whether a subject is at risk of developing lupus characterized by the higher levels of interferon inducible gene expression compared to control subjects is provided, the method comprising detecting in a biological sample obtained from the subject, the presence of a genetic signature indicative of the risk, wherein said genetic signature comprises a set of at least three single nucleotide polymorphisms (SNPs), each SNP occurring in a SLE risk locus, wherein each SLE risk locus is selected from HLA-DR3, HLA-DR2, TNFSF4, IRAK1, STAT4, UBE2L3, and IRF5.

In yet another aspect, a method of identifying lupus in a subject is provided, the method comprising detecting in a biological sample derived from the subject the presence of a variation in at least one SLE-associated locus as set forth in Table 12, wherein the variation at the at least one locus occurs at a nucleotide position corresponding to the position of a single nucleotide polymorphism (SNP) for the at least one locus as set forth in Table 12, and wherein the subject is suspected of suffering from lupus. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826. In one embodiment, the variation at each locus is a genetic variation. In one embodiment, the variation at the at least one locus comprises a SNP as set forth in Table 12. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In another aspect, a method for predicting responsiveness of a subject with lupus to a lupus therapeutic agent is provided, the method comprising determining whether the subject comprises a variation in at least one SLE-associated locus as set forth in Table 12, wherein the variation at the at least one locus occurs at a nucleotide position corresponding to the position of a single nucleotide polymorphism (SNP) for the at least one locus as set forth in Table 12, wherein the presence of a variation at each locus indicates the responsiveness of the subject to the therapeutic agent. In certain embodiments, the subject comprises a variation in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826. In one embodiment, the variation at each locus is a genetic variation. In one embodiment, the variation at the at least one locus comprises a SNP as set forth in Table 12.

In yet another aspect, a method of diagnosing or prognosing lupus in a subject is provided, the method comprising detecting in a biological sample derived from the subject the presence of a variation in at least one SLE-associated locus as set forth in Table 12, wherein: the biological sample is known to comprise, or suspected of comprising, nucleic acid comprising at least one SLE-associated locus as set forth in Table 12, each locus comprising a variation; the variation at the at least one locus comprises, or is located at a nucleotide position corresponding to, a SNP as set forth in Table 12; and the presence of the variation at the at least one locus is a diagnosis or prognosis of lupus in the subject. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826.

In a still further aspect, a method of aiding in the diagnosis or prognosis of lupus in a subject is provided, the method comprising detecting in a biological sample derived from the subject the presence of a variation in at least one SLE-associated locus as set forth in Table 12, wherein: the biological sample is known to comprise, or suspected of comprising, nucleic acid comprising at least one SLE-associated locus as set forth in Table 12, the at least one locus comprising a variation; the variation at the at least one locus comprises, or is located at a nucleotide position corresponding to, a SNP as set forth in Table 12; and the presence of the variation at the at least one locus is a diagnosis or prognosis of lupus in the subject. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826.

In one aspect, a method of treating a lupus condition in a subject in whom a genetic variation is known to be present at a nucleotide position corresponding to a single nucleotide polymorphism (SNP) as set forth in Table 12 in at least one SLE-associated locus as set forth in Table 12 is provided, the method comprising administering to the subject a therapeutic agent effective to treat the condition.

In another aspect, a method of treating a subject having a lupus condition is provided, the method comprising administering to the subject a therapeutic agent effective to treat the condition in a subject who has a genetic variation at a nucleotide position corresponding to a single nucleotide polymorphism (SNP) as set forth in Table 12 in at least one SLE-associated locus as set forth in Table 12.

In yet another aspect, a method of treating a subject having a lupus condition is provided, the method comprising administering to the subject a therapeutic agent shown to be effective to treat said condition in at least one clinical study wherein the agent was administered to at least five human subjects who each had a genetic variation at a nucleotide position corresponding to a single nucleotide polymorphism (SNP) as set forth in Table 12 in at least one SLE-associated locus as set forth in Table 12.

In one aspect, a method of identifying a subphenotype of lupus in a subject is provided, the method comprising detecting in a biological sample derived from the subject the presence of a variation in at least one SLE-associated locus, wherein the variation at the at least one locus occurs at a nucleotide position corresponding to the position of a single nucleotide polymorphism (SNP) for the at least one locus as set forth in Table 12, and wherein the subject is suspected of suffering from lupus and is suspected of having a subphenotype of lupus. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826. In one embodiment, the variation at the at least one locus is a genetic variation. In one embodiment, the variation at the at least one locus comprises a SNP as set forth in Table 12. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP and Sm. In one embodiment, the biological sample is serum.

In another aspect, a method for predicting responsiveness of a subject with an identified lupus subphenotype to a lupus therapeutic agent is provided, the method comprising determining whether the subject comprises a variation in at least one SLE-associated locus, wherein the variation at the at least one locus occurs at a nucleotide position corresponding to the position of a single nucleotide polymorphism (SNP) for the at least one locus as set forth in Table 12, wherein the presence of a variation at the at least one locus indicates the responsiveness of the subject to the therapeutic agent. In certain embodiments, the subject comprises a variation in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826. In one embodiment, the variation at each locus is a genetic variation. In one embodiment, the variation in the at least one locus comprises a SNP as set forth in Table 12.

In yet another aspect, a method of diagnosing or prognosing a subphenotype of lupus in a subject is provided, the method comprising detecting in a biological sample derived from the subject the presence of a variation in at least one SLE-associated locus as set forth in Table 12, wherein: the biological sample is known to comprise, or suspected of comprising, nucleic acid comprising at least one SLE-associated locus as set forth in Table 12, each locus comprising a variation; the variation at the at least one locus comprises, or is located at a nucleotide position corresponding to, a SNP as set forth in Table 12; and the presence of the variation at the at least one locus is a diagnosis or prognosis of a subphenotype of lupus in the subject. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826. In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP and Sm. In one embodiment, the biological sample is serum.

In a still further aspect, a method of aiding in the diagnosis or prognosis of lupus in a subject, the method comprising detecting in a biological sample derived from the subject the presence of a variation in at least one SLE-associated locus, wherein: the biological sample is known to comprise, or suspected of comprising, nucleic acid comprising at one SLE-associated locus, the at least one locus comprising a variation; the variation at the at least one locus comprises, or is located at a nucleotide position corresponding to, a SNP as set forth in Table 12; and the presence of the variation at the at least one locus is a diagnosis or prognosis of the subphenotype of lupus in the subject. In one embodiment, the subphenotype of lupus is characterized at least in part by the presence of autoantibodies in a biological sample derived from the subject to one or more RNA binding proteins. In one embodiment, the RNA binding protein is selected from SSA, SSB, RNP and Sm. In one embodiment, the biological sample is serum.

In one aspect, a method of treating a lupus condition in a subject in whom a genetic variation is known to be present at a nucleotide position corresponding to a single nucleotide polymorphism (SNP) as set forth in Table 12 in at least one SLE-associated locus as set forth in Table 12, wherein the lupus condition is characterized at least in part by the presence of autoantibodies in a biological sample derived from the subject to one or more RNA binding proteins, the method comprising administering to the subject a therapeutic agent effective to treat the condition.

In another aspect, a method of treating a subject having a lupus condition is provided, the method comprising administering to the subject a therapeutic agent effective to treat the condition in a subject who has a genetic variation at a nucleotide position corresponding to a single nucleotide polymorphism (SNP) as set forth in Table 12 in at least one SLE-associated locus as set forth in Table 12, wherein the lupus condition is characterized at least in part by the presence of autoantibodies in a biological sample derived from the subject to one or more RNA binding proteins.

In yet another aspect, a method of treating a subject having a lupus condition is provided, the method comprising administering to the subject a therapeutic agent shown to be effective to treat said condition in at least one clinical study wherein the agent was administered to at least five human subjects who each had a genetic variation at a nucleotide position corresponding to a single nucleotide polymorphism (SNP) as set forth in Table 12 in at least one SLE-associated locus as set forth in Table 12, wherein the lupus condition is characterized at least in part by the presence of autoantibodies in a biological sample derived from the subject to one or more RNA binding proteins in a biological sample derived from the subject as compared to one or more control subjects.

In a still further aspect, a method of identifying a therapeutic agent effective to treat lupus in a patient subpopulation, the method comprising correlating efficacy of the agent with the presence of a genetic variation at a nucleotide position corresponding to a single nucleotide polymorphism (SNP) as set forth in Table 12 in at least one SLE-associated locus as set forth in Table 12 in the patient subpopulation thereby identifying the agent as effective to treat lupus in said patient subpopulation. In one embodiment, the efficacy of the agent is correlated with the presence of a genetic variation at a nucleotide position corresponding to a SNP as set forth in Table 12 in each of at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826.

In one aspect, a method of treating a lupus subject of a specific lupus patient subpopulation is provided, wherein the subpopulation is characterized at least in part by association with genetic variation at a nucleotide position corresponding to a single nucleotide polymorphism (SNP) as set forth in Table 12 in at least one SLE-associated locus as set forth in Table 12, and wherein the method comprises administering to the subject an effective amount of a therapeutic agent that is approved as a therapeutic agent for said subpopulation. In one embodiment, the subpopulation is characterized at least in part by the presence of autoantibodies to one or more RNA binding proteins, wherein the autoantibodies are capable of being detected in a biological sample. In one embodiment, the RNA binding protein is selected from SSA, SSB, RNP and Sm.

In another aspect, a method comprising manufacturing a lupus therapeutic agent is provided, which includes packaging the agent with instructions to administer the agent to a subject who has or is believed to have lupus and who has a genetic variation at a position corresponding to a single nucleotide polymorphism (SNP) as set forth in Table 12 in at least one SLE-associated locus as set forth in Table 12.

In a further aspect, a method of specifying a therapeutic agent for use in a lupus patient subpopulation is provided, the method comprising providing instructions to administer the therapeutic agent to a patient subpopulation characterized at least in part by a genetic variation at a nucleotide position corresponding to a single nucleotide polymorphism (SNP) as set forth in Table 12 in at least one SLE-associated locus as set forth in Table 12.

In a still further aspect, a method for marketing a therapeutic agent for use in a lupus patient subpopulation is provided, the method comprising informing a target audience about the use of the therapeutic agent for treating the patient subpopulation as characterized at least in part by the presence, in patients of such subpopulation, of a genetic variation at a nucleotide position corresponding to a single nucleotide polymorphism (SNP) as set forth in Table 12 in at least one SLE-associated locus as set forth in Table 12.

In one aspect, a method for selecting a patient suffering from lupus for treatment with a lupus therapeutic agent is provided, the method comprising detecting the presence of a genetic variation at a nucleotide position corresponding to a single nucleotide polymorphism (SNP) as set forth in Table 12 in at least one SLE-associated locus as set forth in Table 12. In certain embodiments, a variation is detected in at least two loci, or at least three loci, or at least four loci, or at least five loci, or at least ten loci, or at 19 loci. In certain embodiments, the at least one SLE-associated locus is selected from GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, Cl₉orf6, and LOC729826. In one embodiment, the variation at the at least one locus is a genetic variation. In one embodiment, the variation at the at least one locus comprises a SNP as set forth Table 12. In one embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay. In one embodiment, the lupus is a subphenotype of lupus characterized at least in part by the presence of autoantibodies in a biological sample derived from the patient to one or more RNA binding proteins for treatment as compared to one or more control subjects. In one embodiment, the RNA binding protein is selected from SSA, SSB, RNP, and Sm.

### BRIEF DESCRIPTION OF THE DRAWINGS

FIG. 1 shows a subset of SLE risk loci associated with anti-RNA binding protein autoantibodies. (A) Allele frequency differences between controls (N=7859) and either RBP-pos SLE cases (total N=487 cases, open symbols) or RBP-neg SLE cases (total N=782 cases, black, filled symbols) are shown for 3 independent case series for 16 confirmed SLE risk alleles. Significant differences in allele frequencies were observed for HLA-DR3, HLA-DR2, TNFSF4, IRAK1, STAT4, UBE2L3 and IRF5. (B) Odds ratios for the combined RBP-pos and RBP-neg subsets are shown together with 95% confidence intervals. (C) Frequencies of RBP-pos (open areas) or RBP-neg (hatched areas) SLE cases are plotted based on the total number of anti-RBP autoAb risk alleles.

FIG. 2 shows an association of anti-RBP autoAb alleles with the interferon (IFN) gene expression signature. IFN gene expression scores in peripheral blood cells were measured using microarrays in 23 healthy controls and 274 SLE cases. The distribution of IFN gene expression composite scores was plotted against the number of anti-RBP risk alleles. Open symbols indicate individuals with serum anti-RBP autoAbs; black, filled symbols indicate individuals lacking serum anti-RBP autoAbs; grey triangles indicate healthy controls. Individuals with 0-1, 2-4, or ≧5 anti-RBP autoAb risk alleles were tested for differences in the distribution of IFN gene expression scores using the Student's T test. The P value for each pairwise group comparison is indicated. The dotted line indicates a threshold of 2 standard deviations above the mean control IFN gene expression score.

### DETAILED DESCRIPTION

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

Primers, oligonucleotides and polynucleotides employed in the present invention can be generated using standard techniques known in the art.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

“Lupus” or “lupus condition”, as used herein is an autoimmune disease or disorder that in general involves antibodies that attack connective tissue. The principal form of lupus is a systemic one, systemic lupus erythematosus (SLE), including cutaneous SLE and subacute cutaneous SLE, as well as other types of lupus (including nephritis, extrarenal, cerebritis, pediatric, non-renal, discoid, and alopecia). See, generally, D'Cruz et al., supra.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′—OH group.

The term “genetic variation” or “nucleotide variation” refers to a change in a nucleotide sequence (e.g., an insertion, deletion, inversion, or substitution of one or more nucleotides, such as a single nucleotide polymorphism (SNP)) relative to a reference sequence (e.g., a commonly-found and/or wild-type sequence, and/or the sequence of a major allele). The term also encompasses the corresponding change in the complement of the nucleotide sequence, unless otherwise indicated. In one embodiment, a genetic variation is a somatic polymorphism. In one embodiment, a genetic variation is a germline polymorphism.

A “single nucleotide polymorphism”, or “SNP”, refers to a single base position in DNA at which different alleles, or alternative nucleotides, exist in a population. The SNP position is usually preceded by and followed by highly conserved sequences of the allele (e.g., sequences that vary in less than 1/100 or 1/1000 members of the populations). An individual may be homozygous or heterozygous for an allele at each SNP position.

The term “amino acid variation” refers to a change in an amino acid sequence (e.g., an insertion, substitution, or deletion of one or more amino acids, such as an internal deletion or an N- or C-terminal truncation) relative to a reference sequence.

The term “variation” refers to either a nucleotide variation or an amino acid variation.

The term “a genetic variation at a nucleotide position corresponding to a SNP,” “a nucleotide variation at a nucleotide position corresponding to a SNP,” and grammatical variants thereof refer to a nucleotide variation in a polynucleotide sequence at the relative corresponding DNA position occupied by said SNP in the genome. The term also encompasses the corresponding variation in the complement of the nucleotide sequence, unless otherwise indicated.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., PCR). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “allele-specific oligonucleotide” refers to an oligonucleotide that hybridizes to a region of a target nucleic acid that comprises a nucleotide variation (generally a substitution). “Allele-specific hybridization” means that, when an allele-specific oligonucleotide is hybridized to its target nucleic acid, a nucleotide in the allele-specific oligonucleotide specifically base pairs with the nucleotide variation. An allele-specific oligonucleotide capable of allele-specific hybridization with respect to a particular nucleotide variation is said to be “specific for” that variation.

The term “allele-specific primer” refers to an allele-specific oligonucleotide that is a primer.

The term “primer extension assay” refers to an assay in which nucleotides are added to a nucleic acid, resulting in a longer nucleic acid, or “extension product,” that is detected directly or indirectly. The nucleotides can be added to extend the 5′ or 3′ end of the nucleic acid.

The term “allele-specific nucleotide incorporation assay” refers to a primer extension assay in which a primer is (a) hybridized to target nucleic acid at a region that is 3′ or 5′ of a nucleotide variation and (b) extended by a polymerase, thereby incorporating into the extension product a nucleotide that is complementary to the nucleotide variation.

The term “allele-specific primer extension assay” refers to a primer extension assay in which an allele-specific primer is hybridized to a target nucleic acid and extended.

The term “allele-specific oligonucleotide hybridization assay” refers to an assay in which (a) an allele-specific oligonucleotide is hybridized to a target nucleic acid and (b) hybridization is detected directly or indirectly.

The term “5′ nuclease assay” refers to an assay in which hybridization of an allele-specific oligonucleotide to a target nucleic acid allows for nucleolytic cleavage of the hybridized probe, resulting in a detectable signal.

The term “assay employing molecular beacons” refers to an assay in which hybridization of an allele-specific oligonucleotide to a target nucleic acid results in a level of detectable signal that is higher than the level of detectable signal emitted by the free oligonucleotide.

The term “oligonucleotide ligation assay” refers to an assay in which an allele-specific oligonucleotide and a second oligonucleotide are hybridized adjacent to one another on a target nucleic acid and ligated together (either directly or indirectly through intervening nucleotides), and the ligation product is detected directly or indirectly.

The term “target sequence,” “target nucleic acid,” or “target nucleic acid sequence” refers generally to a polynucleotide sequence of interest in which a nucleotide variation is suspected or known to reside, including copies of such target nucleic acid generated by amplification.

The term “detection” includes any means of detecting, including direct and indirect detection.

The term “SLE risk locus” and “confirmed SLE risk locus” refer to the loci indicated in Table 2: HLA-DR3, IRF5, STAT4, ITGAM, BLK, PTTG1, ATG5, TNFSF4, PTPN22, IRAK1, FCGR2A, KIAA1542, UBE2L3, PXK, HLA-DR2, BANK1.

The term “SLE-associated locus” refers to the loci indicated in Table 12: GLG1, MAPKAP1, LOC646841, C6orf103, CPM, NCKAP1L, ASB7, NUMBL, NR3C2, HSPA12A, LOC646187, LOC132817, LOC728073, NCOA4, KIAA1486, FDPSL2B, NDRG3, C19orf6, and LOC729826.

The term “SLE risk allele” and “confirmed SLE risk allele” refer to a variation occurring in a SLE risk locus. Such variations include, but are not limited to, single nucleotide polymorphisms, insertions, and deletions. Certain exemplary SLE risk alleles are indicated in Table 2.

The term “SLE-associated allele” refers to a variation occurring in a SLE-associated locus. Such variations include, but are not limited to, single nucleotide polymorphisms, insertions, and deletions. Certain exemplary SLE-associated alleles are indicated in Table 12.

As used herein, a subject “at risk” of developing lupus may or may not have detectable disease or symptoms of disease, and may or may not have displayed detectable disease or symptoms of disease prior to the treatment methods described herein. “At risk” denotes that a subject has one or more risk factors, which are measurable parameters that correlate with development of lupus, as described herein and known in the art. A subject having one or more of these risk factors has a higher probability of developing lupus than a subject without one or more of these risk factor(s).

The term “diagnosis” is used herein to refer to the identification or classification of a molecular or pathological state, disease or condition. For example, “diagnosis” may refer to identification of a particular type of lupus condition, e.g., SLE. “Diagnosis” may also refer to the classification of a particular sub-type of lupus, e.g., by tissue/organ involvement (e.g., lupus nephritis), by molecular features (e.g., a patient subpopulation characterized by genetic variation(s) in a particular gene or nucleic acid region.)

The term “aiding diagnosis” is used herein to refer to methods that assist in making a clinical determination regarding the presence, or nature, of a particular type of symptom or condition of lupus. For example, a method of aiding diagnosis of lupus can comprise measuring the presence of absence of one or more SLE risk loci or SLE risk alleles in a biological sample from an individual.

The term “prognosis” is used herein to refer to the prediction of the likelihood of autoimmune disorder-attributable disease symptoms, including, for example, recurrence, flaring, and drug resistance, of an autoimmune disease such as lupus. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc., or whether long-term survival of the patient, following a therapeutic regimen is likely. Diagnosis of SLE may be according to current American College of Rheumatology (ACR) criteria. Active disease may be defined by one British Isles Lupus Activity Group's (BILAG) “A” criteria or two BILAG “B” criteria. Some signs, symptoms, or other indicators used to diagnose SLE adapted from: Tan et al. “The Revised Criteria for the Classification of SLE” Arth Rheum 25 (1982) may be malar rash such as rash over the cheeks, discoid rash, or red raised patches, photosensitivity such as reaction to sunlight, resulting in the development of or increase in skin rash, oral ulcers such as ulcers in the nose or mouth, usually painless, arthritis, such as non-erosive arthritis involving two or more peripheral joints (arthritis in which the bones around the joints do not become destroyed), serositis, pleuritis or pericarditis, renal disorder such as excessive protein in the urine (greater than 0.5 gm/day or 3+ on test sticks) and/or cellular casts (abnormal elements derived from the urine and/or white cells and/or kidney tubule cells), neurologic signs, symptoms, or other indicators, seizures (convulsions), and/or psychosis in the absence of drugs or metabolic disturbances that are known to cause such effects, and hematologic signs, symptoms, or other indicators such as hemolytic anemia or leukopenia (white bloodcount below 4,000 cells per cubic millimeter) or lymphopenia (less than 1,500 lymphocytes per cubic millimeter) or thrombocytopenia (less than 100,000 platelets per cubic millimeter). The leukopenia and lymphopenia generally must be detected on two or more occasions. The thrombocytopenia generally must be detected in the absence of drugs known to induce it. The invention is not limited to these signs, symptoms, or other indicators of lupus.

As used herein, “treatment” refers to clinical intervention in an attempt to alter the natural course of the individual or cell being treated, and can be performed before or during the course of clinical pathology. Desirable effects of treatment include preventing the occurrence or recurrence of a disease or a condition or symptom thereof, alleviating a condition or symptom of the disease, diminishing any direct or indirect pathological consequences of the disease, decreasing the rate of disease progression, ameliorating or palliating the disease state, and achieving remission or improved prognosis. In some embodiments, methods and compositions of the invention are useful in attempts to delay development of a disease or disorder.

An “effective amount” refers to an amount effective, at dosages and for periods of time necessary, to achieve the desired therapeutic or prophylactic result. A “therapeutically effective amount” of a therapeutic agent may vary according to factors such as the disease state, age, sex, and weight of the individual, and the ability of the antibody to elicit a desired response in the individual. A therapeutically effective amount is also one in which any toxic or detrimental effects of the therapeutic agent are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

A “patient subpopulation,” and grammatical variations thereof, as used herein, refers to a patient subset characterized as having one or more distinctive measurable and/or identifiable characteristics that distinguishes the patient subset from others in the broader disease category to which it belongs. Such characteristics include disease subcategories (e.g., SLE, lupus nephritis), gender, lifestyle, health history, organs/tissues involved, treatment history, etc. In one embodiment, a patient subpopulation is characterized by genetic signatures, including genetic variations in particular nucleotide positions and/or regions (such as SNPs).

A “control subject” refers to a healthy subject who has not been diagnosed as having lupus or a lupus condition and who does not suffer from any sign or symptom associated with lupus or a lupus condition.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. A “reference sample”, “reference cell”, “reference tissue”, “control sample”, “control cell”, or “control tissue”, as used herein, refers to a sample, cell or tissue obtained from a source known, or believed, not to be afflicted with the disease or condition for which a method or composition of the invention is being used to identify. In one embodiment, a reference sample, reference cell, reference tissue, control sample, control cell, or control tissue is obtained from a healthy part of the body of the same subject or patient in whom a disease or condition is being identified using a composition or method of the invention. In one embodiment, a reference sample, reference cell, reference tissue, control sample, control cell, or control tissue is obtained from a healthy part of the body of an individual who is not the subject or patient in whom a disease or condition is being identified using a composition or method of the invention.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis according to the present invention, provided that it is understood that the present invention comprises a method whereby the same section of tissue sample is analyzed at both morphological and molecular levels, or is analyzed with respect to both protein and nucleic acid.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of gene expression analysis or protocol, one may use the results of the gene expression analysis or protocol to determine whether a specific therapeutic regimen should be performed.

The word “label” when used herein refers to a compound or composition which is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable.

A “medicament” is an active drug to treat a disease, disorder, and/or condition. In one embodiment, the disease, disorder, and/or condition is lupus or its symptoms or side effects.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

A “lupus therapeutic agent”, a “therapeutic agent effective to treat lupus”, and grammatical variations thereof, as used herein, refer to an agent that when provided in an effective amount is known, clinically shown, or expected by clinicians to provide a therapeutic benefit in a subject who has lupus. In one embodiment, the phrase includes any agent that is marketed by a manufacturer, or otherwise used by licensed clinicians, as a clinically-accepted agent that when provided in an effective amount would be expected to provide a therapeutic effect in a subject who has lupus. In one embodiment, a lupus therapeutic agent comprises a non-steroidal anti-inflammatory drug (NSAID), which includes acetylsalicylic acid (e.g., aspirin), ibuprofen (Motrin), naproxen (Naprosyn), indomethacin (Indocin), nabumetone (Relafen), tolmetin (Tolectin), and any other embodiments that comprise a therapeutically equivalent active ingredient(s) and formulation thereof. In one embodiment, a lupus therapeutic agent comprises acetaminophen (e.g., Tylenol), corticosteroids, or anti-malarials3 (e.g., chloroquine, hydroxychloroquine). In one embodiment, a lupus therapeutic agent comprises an immunomodulating drug (e.g., azathioprine, cyclophosphamide, methotrexate, cyclosporine). In one embodiment, a lupus therapeutic agent is an anti-B cell agent (e.g., anti-CD20 (e.g., rituximab), anti-CD22), an anti-cytokine agent (e.g., anti-tumor necrosis factor α, anti-interleukin-1-receptor (e.g., anakinra), anti-interleukin 10, anti-interleukin 6 receptor, anti-interferon alpha, anti-B-lymphocyte stimulator), an inhibitor of costimulation (e.g., anti-CD154, CTLA4-Ig (e.g., abatacept)), a modulator of B-cell anergy (e.g., LW 394 (e.g., abetimus)). In one embodiment, a lupus therapeutic agent comprises hormonal treatment (e.g., DHEA), and anti-hormonal therapy (e.g., the anti-prolactin agent bromocriptine). In one embodiment, a lupus therapeutic agent is an agent that provides immunoadsorption, is an anti-complement factor (e.g., anti-C5a), T cell vaccination, cell transfection with T-cell receptor zeta chain, or peptide therapies (e.g., edratide targeting anti-DNA idiotypes).

A therapeutic agent that has “marketing approval”, or that has been “approved as a therapeutic agent”, or grammatical variations thereof of these phrases, as used herein, refer to an agent (e.g., in the form of a drug formulation, medicament) that is approved, licensed, registered or authorized by a relevant governmental entity (e.g., federal, state or local regulatory agency, department, bureau) to be sold by and/or through and/or on behalf of a commercial entity (e.g., a for-profit entity) for the treatment of a particular disorder (e.g., lupus) or a patient subpopulation (e.g., patients with lupus nephritis, patients of a particular ethnicity, gender, lifestyle, disease risk profile, etc.). A relevant governmental entity includes, for example, the Food and Drug Administration (FDA), European Medicines Evaluation Agency (EMEA), and equivalents thereof.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is a minimum antibody fragment which contains a complete antigen-binding site. In one embodiment, a two-chain Fv species consists of a dimer of one heavy- and one light-chain variable domain in tight, non-covalent association. Collectively, the six CDRs of an Fv confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6855-9855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 55-93 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “affinity matured” antibody is one with one or more alterations in one or more CDRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking antibody” or an “antagonist antibody” is one which inhibits or reduces a biological activity of the antigen it binds. Certain blocking antibodies or antagonist antibodies partially or completely inhibit the biological activity of the antigen.

A “small molecule” or “small organic molecule” is defined herein as an organic molecule having a molecular weight below about 500 Daltons.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At-211, Cu-67, Bi-212, and Pd-109.

An “isolated” biological molecule, such as a nucleic acid, polypeptide, or antibody, is one which has been identified and separated and/or recovered from at least one component of its natural environment.

Reference to “about” a value or parameter herein includes (and describes) embodiments that are directed to that value or parameter per se. For example, description referring to “about X” includes description of “X.”

### General Techniques

Nucleotide variations associated with lupus are provided herein. These variations provide biomarkers for lupus, and/or predispose or contribute to development, persistence and/or progression of lupus. Accordingly, the invention disclosed herein is useful in a variety of settings, e.g., in methods and compositions related to lupus diagnosis and therapy.

### Detection of Genetic Variations

Nucleic acid, according to any of the above methods, may be genomic DNA; RNA transcribed from genomic DNA; or cDNA generated from RNA. Nucleic acid may be derived from a vertebrate, e.g., a mammal. A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. The amplicons may then be subjected to a variation detection method, such as those described below, to determine whether a variation is present in the amplicon.

Variations may be detected by certain methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495); analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from tumor tissue. The nucleic acid sequence of the amplified sequences can then be determined and variations identified therefrom. Amplification techniques are well known in the art, e.g., polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. See, e.g., Wu et al., Genomics 4:560-569 (1989). In addition, a technique known as allele-specific PCR can also be used to detect variations (e.g., substitutions). See, e.g., Ruano and Kidd (1989) Nucleic Acids Research 17:8392; McClay et al. (2002) Analytical Biochem. 301:200-206. In certain embodiments of this technique, an allele-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) allele-specific nucleotide incorporation assays, such as single base extension assays (see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316); (2) allele-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′ nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. One example of a mismatch detection method is the Mismatch Repair Detection (MRD) assay described, e.g., in Faham et al., Proc. Natl. Acad. Sci. USA 102:14717-14722 (2005) and Faham et al., Hum. Mol. Genet. 10:1657-1664 (2001). Another example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. See, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. See, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. See, e.g. Orita et al., Proc. Natl. Acad. Sci. USA 86:2766-2770, 1989, and Genomics, 5:874-879, 1989.

A biological sample may be obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Variations in target nucleic acids (or encoded polypeptides) may be detected from a tumor sample or from other body samples such as urine, sputum or serum. (Cancer cells are sloughed off from tumors and appear in such body samples.) By screening such body samples, a simple early diagnosis can be achieved for diseases such as cancer. In addition, the progress of therapy can be monitored more easily by testing such body samples for variations in target nucleic acids (or encoded polypeptides). Additionally, methods for enriching a tissue preparation for tumor cells are known in the art. For example, the tissue may be isolated from paraffin or cryostat sections. Cancer cells may also be separated from normal cells by flow cytometry or laser capture microdissection.

Subsequent to the determination that a subject, or the tissue or cell sample comprises a genetic variation disclosed herein, it is contemplated that an effective amount of an appropriate lupus therapeutic agent may be administered to the subject to treat the lupus condition in the subject. Diagnosis in mammals of the various pathological conditions described herein can be made by the skilled practitioner. Diagnostic techniques are available in the art which allow, e.g., for the diagnosis or detection of lupus in a mammal.

A lupus therapeutic agent can be administered in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering lupus therapeutic agents may be determined empirically, and making such determinations is within the skill in the art. Single or multiple dosages may be employed. For example, an effective dosage or amount of interferon inhibitor used alone may range from about 1 mg/kg to about 100 mg/kg of body weight or more per day. Interspecies scaling of dosages can be performed in a manner known in the art, e.g., as disclosed in Mordenti et al., Pharmaceut. Res., 8:1351 (1991).

When in vivo administration of a lupus therapeutic agent is employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, may necessitate delivery in a manner different from that to another organ or tissue.

It is contemplated that yet additional therapies may be employed in the methods. The one or more other therapies may include but are not limited to, administration of steroids and other standard of care regimens for the disorder in question. It is contemplated that such other therapies may be employed as an agent separate from, e.g., a targeted lupus therapeutic agent.

Methods of detecting the presence of lupus by detecting a variation in one or more SLE risk loci and/or one or more SLE-associated loci derived from a biological sample are provided. In one embodiment, the biological sample is obtained from a mammal suspected of having lupus.

Methods of determining the genotype of a biological sample is provided by detecting whether a genetic variation is present in one or more SLE risk locus and/or SLE-associated locus derived from the biological sample are provided. In one embodiment, the genetic variation is at a nucleotide position corresponding to the position of a SNP set forth in Table 2. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one embodiment, the genetic variation is at a nucleotide position corresponding to the position of a SNP set forth in Table 12. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In another embodiment, the biological sample is known to comprise, or suspected of comprising, nucleic acid comprising one or more SLE risk loci and/or one or more SLE-associated loci, each locus comprising a variation. In another embodiment, the biological sample is a cell line, e.g., a primary or immortalized cell line. In one such embodiment, the genotyping provides a basis for classifying or sub-classifying disease.

Also provided are methods for diagnosing lupus in a mammal by detecting the presence of one or more variations in nucleic acid comprising one or more SLE risk loci and/or one more SLE-associated loci derived from a biological sample obtained from the mammal, wherein the biological sample is known to comprise, or suspected of comprising, nucleic acid comprising one or more SLE risk loci, or one or more SLE-associated loci, each locus comprising a variation. Also provided are methods for aiding in the diagnosing lupus in a mammal by detecting the presence of one or more variations in nucleic acid comprising one or more SLE risk loci and/or one more SLE-associated loci derived from a biological sample obtained from the mammal, wherein the biological sample is known to comprise, or suspected of comprising, nucleic acid comprising one or more SLE risk loci and/or one more SLE-associated loci, each locus comprising a variation. In one embodiment, the variation is a genetic variation. In one embodiment, the genetic variation is at a nucleotide position corresponding to the position of a SNP set forth in Table 2. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one embodiment, the genetic variation is at a nucleotide position corresponding to the position of a SNP set forth in Table 12. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

In another embodiment, a method is provided for predicting whether a subject with lupus will respond to a therapeutic agent by determining whether the subject comprises a variation in one or more SLE risk loci as set forth in Table 2, and/or one or more SLE-associated loci as set forth in Table 12, wherein the variation at each locus occurs at a nucleotide position corresponding to the position of a single nucleotide polymorphism (SNP) for each of the loci as set forth in Table 2 or in Table 12, respectively, wherein the presence of a variation at each locus indicates that the subject will respond to the therapeutic agent. In one embodiment, the variation is a genetic variation. In one embodiment, the genetic variation is at a nucleotide position corresponding to the position of a SNP set forth in Table 2. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one embodiment, the genetic variation is at a nucleotide position corresponding to the position of a SNP set forth in Table 12. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Also provided are methods for assessing predisposition of a subject to develop lupus by detecting presence or absence in the subject of a variation in one or more SLE risk loci as set forth in Table 2, and/or one or more SLE-associated loci as set forth in Table 12, wherein the variation at each locus occurs at a nucleotide position corresponding to the position of a single nucleotide polymorphism (SNP) for each of the loci as set forth in Table 2 or in Table 12, respectively, wherein the presence of a variation at each locus indicates that the subject is predisposed to develop lupus. In one embodiment, the variation is a genetic variation. In one embodiment, the genetic variation is at a nucleotide position corresponding to the position of a SNP set forth in Table 2. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one embodiment, the genetic variation is at a nucleotide position corresponding to the position of a SNP set forth in Table 12. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Also provided are methods of sub-classifying lupus in a mammal, the method comprising detecting the presence of a variation in one or more SLE risk loci as set forth in Table 2, and/or one or more SLE-associated loci as set forth in Table 12, wherein the variation at each locus occurs at a nucleotide position corresponding to the position of a single nucleotide polymorphism (SNP) for each of the loci as set forth in Table 2 or in Table 12, respectively, in a biological sample derived from the mammal, wherein the biological sample is known to comprise, or suspected of comprising, nucleic acid comprising the variation. In one embodiment, the variation is a genetic variation. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In one embodiment, the subclassification is characterized by tissue/organ involvement (e.g., lupus nephritis), gender, and/or ethnicity.

In one embodiment of the detection methods of the invention, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

Also provided are methods of identifying a therapeutic agent effective to treat lupus in a patient subpopulation, the method comprising correlating efficacy of the agent with the presence of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5 in the patient subpopulation, thereby identifying the agent as effective to treat lupus in said patient subpopulation. In one embodiment, the genetic variation is at a nucleotide position corresponding to the position of a SNP set forth in Table 2. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene.

Also provided are methods of identifying a therapeutic agent effective to treat lupus in a patient subpopulation, the method comprising correlating efficacy of the agent with the presence of a genetic variation at a nucleotide position corresponding to a single nucleotide polymorphism (SNP) as set forth in Table 12 in at least one SLE-associated locus as provided in Table 12 in the patient subpopulation, thereby identifying the agent as effective to treat lupus in said patient subpopulation. In one embodiment, the genetic variation is at a nucleotide position corresponding to the position of a SNP set forth in Table 12. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Additional methods provide information useful for determining appropriate clinical intervention steps, if and as appropriate. Therefore, in one embodiment of a method of the invention, the method further comprises a clinical intervention step based on results of the assessment of the presence or absence of a variation in one or more SLE risk loci and/or SLE-associated loci as disclosed herein. For example, appropriate intervention may involve prophylactic and treatment steps, or adjustment(s) of any then-current prophylactic or treatment steps based on genetic information obtained by a method of the invention.

As would be evident to one skilled in the art, in any method described herein, while detection of presence of a variation would positively indicate a characteristic of a disease (e.g., presence or subtype of a disease), non-detection of a variation would also be informative by providing the reciprocal characterization of the disease.

Also provided are methods of amplifying a nucleic acid comprising a SLE risk locus or fragment thereof, wherein the SLE risk locus or fragment thereof comprises a genetic variation. Also provided are methods of amplifying a nucleic acid comprising a SLE-associated locus or fragment thereof, wherein the SLE-associated locus or fragment thereof comprises a genetic variation. In one embodiment, the method comprises (a) contacting the nucleic acid with a primer that hybridizes to a sequence 5′ or 3′ of the genetic variation, and (b) extending the primer to generate an amplification product comprising the genetic variation. In one embodiment, the method further comprises contacting the amplification product with a second primer that hybridizes to a sequence 5′ or 3′ of the genetic variation, and extending the second primer to generate a second amplification product. In one such embodiment, the method further comprises amplifying the amplification product and second amplification product, e.g., by polymerase chain reaction.

In some embodiments, the genetic variation is at a nucleotide position corresponding to the position of a SNP of the present invention. In one such embodiment, the genetic variation comprises a SNP set forth in Table 2. In one embodiment, the genetic variation is in genomic DNA that encodes a gene (or its regulatory region), wherein the gene (or its regulatory region) comprises a SNP set forth in Table 2. In one such embodiment, the genetic variation comprises a SNP set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Still further methods include methods of treating lupus in a mammal, comprising steps of obtaining tissue or a cell sample from the mammal, examining the tissue or cells for presence or absence of a variation as disclosed herein, and upon determining presence or absence of the variation in said tissue or cell sample, administering an effective amount of an appropriate therapeutic agent to said mammal. Optionally, the methods comprise administering an effective amount of a targeted lupus therapeutic agent, and, optionally, a second therapeutic agent (e.g., steroids, etc.) to said mammal.

Also provided are methods of treating a lupus condition in a subject in whom a genetic variation is known to be present at a nucleotide position corresponding to a single nucleotide polymorphism (SNP) listed in Table 2 in one or more SLE risk loci listed in Table 2, the method comprising administering to the subject a therapeutic agent effective to treat the condition. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene.

Also provided are methods of treating a lupus condition in a subject in whom a genetic variation is known to be present at a nucleotide position corresponding to a single nucleotide polymorphism (SNP) listed in Table 12 in one or more SLE-associated loci listed in Table 12, the method comprising administering to the subject a therapeutic agent effective to treat the condition. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Also provided are methods of treating a subject having a lupus condition, the method comprising administering to the subject a therapeutic agent known to be effective to treat the condition in a subject who has a genetic variation at a nucleotide position corresponding to a single nucleotide polymorphism (SNP) listed in Table 2 in one or more SLE risk loci listed in Table 2. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene.

Also provided are methods of treating a subject having a lupus condition, the method comprising administering to the subject a therapeutic agent known to be effective to treat the condition in a subject who has a genetic variation at a nucleotide position corresponding to a single nucleotide polymorphism (SNP) listed in Table 12 in one or more SLE-associated loci listed in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Also provided are methods of treating a subject having a lupus condition, the method comprising administering to the subject a therapeutic agent previously shown to be effective to treat said condition in at least one clinical study wherein the agent was administered to at least five human subjects who each had a genetic variation at a nucleotide position corresponding to a single nucleotide polymorphism (SNP) listed in Table 2 in one or more SLE risk loci listed in Table 2. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene. In one embodiment, the at least five subjects had two or more different SNPs in total for the group of at least five subjects. In one embodiment, the at least five subjects had the same SNP for the entire group of at least five subjects.

Also provided are methods of treating a subject having a lupus condition, the method comprising administering to the subject a therapeutic agent previously shown to be effective to treat said condition in at least one clinical study wherein the agent was administered to at least five human subjects who each had a genetic variation at a nucleotide position corresponding to a single nucleotide polymorphism (SNP) listed in Table 12 in one or more SLE-associated loci listed in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In one embodiment, the at least five subjects had two or more different SNPs in total for the group of at least five subjects. In one embodiment, the at least five subjects had the same SNP for the entire group of at least five subjects.

Also provided are methods of treating a lupus subject who is of a specific lupus patient subpopulation comprising administering to the subject an effective amount of a therapeutic agent that is approved as a therapeutic agent for said subpopulation, wherein the subpopulation is characterized at least in part by association with genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene. In one embodiment, the subpopulation is of European ancestry. In one embodiment, the invention provides a method comprising manufacturing a lupus therapeutic agent, and packaging the agent with instruction to administer the agent to a subject who has or is believed to have lupus and who has a genetic variation at a position corresponding to a single nucleotide polymorphism (SNP) listed in Table 2. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene.

Also provided are methods of treating a lupus subject who is of a specific lupus patient subpopulation comprising administering to the subject an effective amount of a therapeutic agent that is approved as a therapeutic agent for said subpopulation, wherein the subpopulation is characterized at least in part by association with genetic variation at a nucleotide position corresponding to a single nucleotide polymorphism (SNP) as set forth in Table 12 in at least one SLE-associated locus as provided in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In one embodiment, the invention provides a method comprising manufacturing a lupus therapeutic agent, and packaging the agent with instruction to administer the agent to a subject who has or is believed to have lupus and who has a genetic variation at a position corresponding to a single nucleotide polymorphism (SNP) listed in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

Also provided are methods of specifying a therapeutic agent for use in a lupus patient subpopulation, the method comprising providing instructions to administer the therapeutic agent to a patient subpopulation characterized at least in part by a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene. In one embodiment, the subpopulation is of European ancestry.

Also provided are methods of specifying a therapeutic agent for use in a lupus patient subpopulation, the method comprising providing instructions to administer the therapeutic agent to a patient subpopulation characterized at least in part by a genetic variation at a nucleotide position corresponding to a single nucleotide polymorphism (SNP) as set forth in Table 12 in at least one SLE-associated locus as provided in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In one embodiment, the subpopulation is of European ancestry.

Also provided are methods for marketing a therapeutic agent for use in a lupus patient subpopulation, the method comprising informing a target audience about the use of the therapeutic agent for treating the patient subpopulation as characterized at least in part by the presence, in patients of such subpopulation, of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene. In an embodiment of any of the above methods that comprise the use of a therapeutic agent, such agent comprises a lupus therapeutic agent as disclosed herein.

Also provided are methods for marketing a therapeutic agent for use in a lupus patient subpopulation, the method comprising informing a target audience about the use of the therapeutic agent for treating the patient subpopulation as characterized at least in part by the presence, in patients of such subpopulation, of a genetic variation at a nucleotide position corresponding to a single nucleotide polymorphism (SNP) as set forth in Table 12 in at least one SLE-associated locus as provided in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene. In an embodiment of any of the above methods that comprise the use of a therapeutic agent, such agent comprises a lupus therapeutic agent as disclosed herein.

Also provided are methods for modulating signaling through the type I interferon pathway in a subject in whom a genetic variation is known to be present at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5, the method comprising administering to the subject a therapeutic agent effective to modulate gene expression of one or more interferon inducible genes.

Also provided are methods for selecting a patient suffering from lupus for treatment with a lupus therapeutic agent comprising detecting the presence of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5. In one embodiment, the variation comprises a SNP as set forth in Table 2. In one embodiment, the genetic variation is in genomic DNA that encodes a gene (or its regulatory region), wherein the gene (or its regulatory region) comprises a SNP set forth in Table 2. In one embodiment, the SNP is in a non-coding region of the gene. In one embodiment, the SNP is in a coding region of the gene.

Also provided are methods for selecting a patient suffering from lupus for treatment with a lupus therapeutic agent comprising detecting the presence of a genetic variation at a nucleotide position corresponding to a single nucleotide polymorphism (SNP) as set forth in Table 12 in at least one SLE-associated locus as provided in Table 12. In one embodiment, the variation comprises a SNP as set forth in Table 12. In one embodiment, the genetic variation is in genomic DNA that encodes a gene (or its regulatory region), wherein the gene (or its regulatory region) comprises a SNP set forth in Table 12. In one embodiment, the SNP is in a non-coding region of the gene. In one embodiment, the SNP is in a coding region of the gene.

### Kits

In one embodiment of the invention, kits are provided. In one embodiment, a kit comprises any of the polynucleotides described herein, optionally with an enzyme. In one embodiment, the enzyme is at least one enzyme selected from a nuclease, a ligase, and a polymerase.

In one embodiment, the invention provides a kit comprising a composition of the invention, and instructions for using the composition to detect lupus by determining whether a subject's genome comprises a genetic variation as disclosed herein. In one embodiment, the composition of the invention comprises a plurality of polynucleotides capable of specifically hybridizing to one more SLE risk loci as set forth in Table 2, each SLE risk locus comprising a genetic variation at a nucleotide position corresponding to the position of a SNP set forth in Table 2, or complements thereof. In one embodiment, the composition of the invention comprises nucleic acid primers capable of binding to and effecting polymerization (e.g., amplification) of at least a portion of SLE risk locus. In one embodiment, the composition of the invention comprises a binding agent (e.g., primer, probe) that specifically detects a polynucleotide comprising a SLE risk locus (or complement thereof). In one embodiment, the invention provides an article of manufacture comprising a therapeutic agent, combined with instructions to use the agent to treat a lupus patient who has a variation in one or more SLE risk loci as disclosed herein.

Also provided are kits comprising a composition of the invention, and instructions for using the composition to detect lupus by determining whether a subject's genome comprises a genetic variation as disclosed herein. In one embodiment, the composition of the invention comprises a plurality of polynucleotides capable of specifically hybridizing to one more SLE-associated loci as set forth in Table 12, each SLE-associated locus comprising a genetic variation at a nucleotide position corresponding to the position of a SNP set forth in Table 12, or complements thereof. In one embodiment, the composition of the invention comprises nucleic acid primers capable of binding to and effecting polymerization (e.g., amplification) of at least a portion of a SLE-associated locus. In one embodiment, the composition of the invention comprises a binding agent (e.g., primer, probe) that specifically detects a polynucleotide comprising a SLE-associated locus (or complement thereof). In one embodiment, the invention provides an article of manufacture comprising a therapeutic agent, combined with instructions to use the agent to treat a lupus patient who has a variation in one or more SLE-associated loci as disclosed herein.

For use in the applications described or suggested above, kits or articles of manufacture are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a polynucleotide comprising a SLE risk locus or a SLE-associated locus. Where the kit utilizes nucleic acid hybridization to detect the target nucleic acid, the kit may also have containers containing nucleotide(s) for amplification of the target nucleic acid sequence and/or a container comprising a reporter means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The kits of the invention have a number of embodiments. A typical embodiment is a kit comprising a container, a label on said container, and a composition contained within said container; wherein the composition includes detecting agent for a polynucleotide comprising a SLE risk locus and/or SLE-associated locus, the label on said container indicates that the composition can be used to evaluate the presence of the polynucleotide comprising a SLE risk locus and/or SLE-associated locus in at least one type of mammalian cell, and instructions for using the detecting agent for evaluating the presence of the polynucleotide comprising a SLE risk locus and/or SLE-associated locus in at least one type of mammalian cell. The kit can further comprise a set of instructions and materials for preparing a tissue sample and applying antibody and probe to the same section of a tissue sample. For example, a kit may comprise a container, a label on said container, and a composition contained within said container; wherein the composition includes a polynucleotide that hybridizes to a complement of a polynucleotide comprising a SLE risk locus or SLE-associated locus under stringent conditions, the label on said container indicates that the composition can be used to evaluate the presence of a polynucleotide comprising a SLE risk locus or SLE-associated locus in at least one type of mammalian cell, and instructions for using the polynucleotide for evaluating the presence of a polynucleotide comprising a SLE risk locus or SLE-associated locus in at least one type of mammalian cell.

Other optional components in the kit include one or more buffers (e.g., block buffer, wash buffer, substrate buffer, etc), other reagents such as substrate (e.g., chromogen) which is chemically altered by an enzymatic label, epitope retrieval solution, control samples (positive and/or negative controls), control slide(s) etc.

### Methods of Marketing

The invention herein also encompasses a method for marketing a lupus therapeutic agent or a pharmaceutically acceptable composition thereof comprising promoting to, instructing, and/or specifying to a target audience, the use of the agent or pharmaceutical composition thereof for treating a patient or patient population with lupus from which a sample has been obtained showing the presence of a genetic variation as disclosed herein.

Marketing is generally paid communication through a non-personal medium in which the sponsor is identified and the message is controlled. Marketing for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The marketing of the diagnostic method herein may be accomplished by any means. Examples of marketing media used to deliver these messages include television, radio, movies, magazines, newspapers, the internet, and billboards, including commercials, which are messages appearing in the broadcast media.

The type of marketing used will depend on many factors, for example, on the nature of the target audience to be reached, e.g., hospitals, insurance companies, clinics, doctors, nurses, and patients, as well as cost considerations and the relevant jurisdictional laws and regulations governing marketing of medicaments and diagnostics. The marketing may be individualized or customized based on user characterizations defined by service interaction and/or other data such as user demographics and geographical location.

The following are examples of the methods and compositions of the invention. It is understood that various other embodiments may be practiced, given the general description provided above.

### EXAMPLES

Throughout the Examples, references to certain publications are denoted by numbers, which have complete bibliography information at the end of the Examples section.

### Example 1

### Identification of Confirmed SLE Risk Loci and SLE Risk Alleles

The selection and genotyping of SLE cases, as well as controls from the New York Health Project (NYHP) collection (Mitchell et al., J Urban Health 81(2):301-10 (2004)), were described previously (Horn et al., N Engl J Med 358(9):900-9 (2008)). As detailed below, the SLE cases consisted of three case series: a) 338 cases from the Autoimmune Biomarkers Collaborative Network (ABCoN) (Bauer et al., PLoS medicine 3(12):e491 (2006)), an NIH/NIAMS-funded repository, and 141 cases from the Multiple Autoimmune Disease Genetics Consortium (MADGC) (Criswell et al., Am J Hum Genet. 76(4):561-71 (2005)); b) 613 cases from the University of California San Francisco (UCSF) Lupus Genetics Project (Seligman et al., Arthritis Rheum 44(3):618-25 (2001); Remmers et al., N Engl J Med 357(10):977-86 (2007)); and c) 335 cases from the University of Pittsburgh Medical Center (UPMC) (Demirci et al., Ann Hum Genet. 71(Pt 3):308-11 (2007)) and 8 cases from The Feinstein Institute for Medical Research. The controls were 1861 samples from the NYHP collection, 1722 samples from the publicly available iControlDB database (available at Illumina Inc.), and 4564 samples from the publicly available National Cancer Institute Cancer Genetic Markers of Susceptibility (CGEMS) project (available on the world-wide web at cgems.cancer.gov).

### Genomewide Data Set of 1310 SLE Cases and 7859 Controls

We previously described the selection and genotyping of SLE case samples (Horn et al., N Engl J Med 358(9):900-9 (2008)). All SLE cases were North Americans of European descent, as determined by self-report and confirmed by genotyping. The diagnosis of SLE (fulfillment of four or more of the American College of Rheumatology [ACR] defined criteria [Hochberg et al., Arthritis Rheum 40(9):1725[1997]]) was confirmed in all cases by medical record review (94%) or through written documentation of criteria by treating rheumatologists (6%). Clinical data for these case series are presented elsewhere (Seligman et al., Arthritis Rheum 44(3):618-25 (2001); Criswell et al., Am J Hum Genet. 76(4):561-71 (2005); Bauer et al., PLoS medicine 3(12):e491 (2006); Demirci et al., Ann Hum Genet. 71(Pt 3):308-11 (2007); Remmers et al., N Engl J Med 357(10):977-86 (2007)). Genotyping and selection of the NYHP samples was described previously (Hom et al., N Engl J Med 358(9):900-9 (2008)).

Sample and SNP filtering was conducted using analytical modules within the software programs PLINK and EIGENSTRAT as described below (see also Purcell et al., Am J Hum Genet. 81(3):559-75 (2007); Price et al., Nat Genet. 38(8):904-09 (2006)). The genomewide SNP data were used in this study to facilitate close matching of cases and controls, and to provide genotypes at the confirmed and suspected SLE loci.

a) SLE Cases, NYHP Samples, and iControlDB Samples

The Illumina 550K SNP array, version 1 (HH550v1) was used to genotype 464 cases and 1962 controls, and the Illumina 550K SNP array, version 3 (HH550v3) was used to genotype 971 cases and 1621 controls as described previously (Horn et al., N Engl J Med 358(9):900-9 (2008)). Samples where the reported sex did not match the observed sex (HH550v1:10, HH550v3:11) and samples with >5% missing genotypes (HH550v1:25, HH550v3:21) were excluded from the analysis. Cryptic relatedness between the SLE cases and controls was determined by the estimation of the identity-by-state (IBS) across the genome for all possible pair-wise sample combinations. A sample from each pair estimated to be duplicates or 1st-3rd degree relatives were excluded (Pi_hat≧0.10 and Z1≧0.15; HH550v1:88, HH550v3:73).

SNPs with HWE P≦1×10⁻⁶ in controls (HH550v1:3176, HH550v3:2240) and SNPs with >5% missing data (HH550v1:12605, HH550v3:7137) were removed. The SNPs were tested for a significant difference in the frequency of missing data between cases and controls, and SNPs with P≦1×10⁻⁵ in the differential missingness test implemented in PLINK were removed (HH550v1:5027, HH550v3:2804). The SNPs were also tested for a significant allele frequency difference between genders; all SNPs had P≧1×10⁻⁹ in controls. The data was examined for the presence of batch effects (for example, between ABCoN samples and all other cases), and SNPs with an allele frequency difference with a P≦1×10−9 were excluded (HH550v1:18, HH550v3:10). Variants with heterozygous haploid genotypes were set to missing (HH550v1:2305, HH550v3:875). In addition, variants with a minor allele frequency <0.0001 were removed (HH550v1:97, HH550v3:57).

### b) CGEMS Samples

For the 2277 prostate cancer samples and, separately, 2287 breast cancer samples, heterozygous haploid genotypes were set to missing (prostate: 2717, breast: 0). Samples where the reported gender did not match the observed gender (prostate: 0, breast: 2) and samples with >5% missing data (prostate: 15, breast: 1) were excluded. Samples were tested for cryptic relatedness, as described above, and we removed one sample from each pair estimated to be duplicates or 1st-3rd degree relatives (Pi_hat≧0.10 and Z1≧0.15; prostate: 12, breast: 7). SNPs with a MAF<0.0001 (prostate: 3254, breast: 2166) were removed.

### c) All Samples

Additional data quality filters were applied to the merged dataset consisting of all SLE cases and controls. SNPs with >5% missing data (N=65,421) and samples with >5% missing data (N=0) were removed. A test for duplicate samples was conducted using 957 independent SNPs with MAF≧0.45, and no duplicate samples were found. SNPs with HWE P≦1×10⁻⁶ in controls (N=2174) and SNPs with >2% missing data (N=5522) were removed. We tested the SNPs for a significant difference in the proportion of missing data between cases and controls and removed SNPs with excess missing data differential (P≦1×10⁻⁵, N=16080). SNPs were tested for a significant difference between genders and all SNPs had P≧1×10⁻⁹ in controls. SNPs were also examined for the presence of batch effects; in particular, between CGEMS breast cancer samples and all other controls, and between CGEMS prostate cancer samples and all other controls and removed SNPs with P<1×10⁻⁹ (N=73). After application of the above quality filters, 480,831 SNPs remained.

The cases and controls were tested for the presence of population outliers using EIGENSTRAT. SNPs with MAF<2% in cases (N=16068), HWE P≦1×10⁻⁴ in controls (N=977), or >1% missing data (N=17029); SNPs in regions of abnormal LD patterns due to structural variation on chromosomes 6 (from 24-36 Mb), 8 (8-12 Mb), 11 (42-58 Mb), and 17 (40-43 Mb); and SNPs in the pseudoautosomal region of chromosome X (N=12) were excluded for the purpose of determining the principal components (EIGENSTRAT) of variation to detect population outliers. Samples with greater than 6 standard deviations from the mean along any of the top 10 principal components were removed (N=148).

The final data set had 1310 cases, 7859 controls, and 480,831 SNPs. The final genomic control inflation factor (λ_{gc})¹⁰ was 1.06, indicating excellent matching of cases and controls.

### Identification of Confirmed SLE Risk Loci and SLE Risk Alleles

We examined the literature relating to SLE risk loci and alleles and applied statistical methods as described herein to identify confirmed SLE risk loci and confirmed SLE risk alleles. In brief, we identified loci with 2 independent published reports in non-overlapping SLE cohorts, each with a P≦1×10⁻⁵. A total of 7 loci fulfilled the requirements (see Table 1). Thus, each of the loci listed in Table 1 is a confirmed SLE risk locus. Table 1 also lists alleles for each of the confirmed SLE risk loci, and accordingly, those are confirmed SLE risk alleles. An additional 18 loci were identified in which a single publication reported an association with a P≦1×10⁻⁵. For 14 of those 18 loci, we found the identical variant or a near-perfect proxy (r²>0.75) in our genomewide data set (described above) of 1310 SLE cases and 7859 matched controls. For those 14 loci, a meta-analysis was performed to combine the reported association and the association in our data set; 9 of the loci achieved a P≦5×10⁻⁸ and thus, we also identified as confirmed SLE risk loci (Table 3). Further details of these analyses are presented below.

SLE Risk Loci and SLE Risk Alleles with 2 Independent Published Reports

We identified loci with 2 independent published reports in non-overlapping SLE cohorts, each with a P≦1×10⁻⁵ (corresponding to a P value of 2.4×10⁻⁹ using Fisher's combined probability test) (Table 1). The identical variant (or proxy with r²>0.3) showing association to SLE with the same direction of effect was required. A total of 7 loci fulfilled the requirements, including the allele HLA-DRB1*0301 (for locus HLA-DR3) (Hartung et al., J Clin Invest 90:1346-51 (1992); Yao et al., Eur J Immunogenet 20(4):259-66 (1993)), the allele HLA-DRB1*1501 (for locus HLA-DR2) (Hartung et al., J Clin Invest 90:1346-51 (1992); Yao et al., Eur J Immunogenet 20(4):259-66 (1993)), and the following loci: Protein Tyrosine Phosphatase Non-receptor type 22 (PTPN22) (Lee et al., Rheumatology (Oxford, England) 46(1):49-56 (2007); Harley et al., Nat Genet. 40(2):204-10 2008)), Interferon Regulatory Factor 5 (IRF5) (Sigurdsson et al., Am J Hum Genet. 76(3):528-37 (2005); Graham et al., Nat Genet. 38(5):550-55 (2006)), Signal Transducer and Activator of Transcription 4 (STAT4) (Remmers et al., N Engl J Med 357(10):977-86 (2007); Harley et al., Nat Genet. 40(2):204-10 (2008)), B Lymphoid tyrosine Kinase (BLK) (Horn et al., N Engl J Med 358(9):900-9 (2008); Harley et al., Nat Genet. 40(2):204-10 (2008) and Integrin Alpha M (ITGAM) (Horn et al., N Engl J Med 358(9):900-9 (2008); Nath et al., Nat Genet. 40(2):152-4 (2008)). The identical allele or best proxy (r²>0.85) in our genomewide data set of 1310 SLE cases and 7859 controls was advanced into the analysis (Table 1).

SLE Risk Loci and SLE Risk Alleles with 1 Published Report

An additional 18 loci were identified in which there was a single publication reporting an association with a P≦1×10⁻⁵ (Prokunina et al., Nat Genet. 32(4):666-9 (2002); Sigurdsson et al., Am J Hum Genet. 76(3):528-37 (2005); Jacob et al., Arthritis Rheum 56(12):4164-73 (2007); Cunninghame Graham et al., Nat Genet. 40(1):83-89 (2008); Edberg et al., Hum Mol Genet. 17(8):1147-55 (2008); Harley et al., Nat Genet. 40(2):204-10 (2008); Kozyrev et al., Nat Genet. 40(2):211-6 (2008); Oishi et al., Journal of human genetics 53(2):151-62 (2008); Sawalha et al., PLoS ONE 3(3):e1727 (2008)). In 14 of the loci, the identical variant or a near-perfect proxy (r²>0.75) was genotyped in our genomewide data set of 1310 SLE cases and 7859 controls (Table 3). A meta-analysis using the methodology described below was performed for the 14 loci, and 9 of the loci achieved a P≦5×10⁻⁸. The loci (labeled by a single gene within the locus) achieving genomewide significance include; Pituitary Tumor-Transforming Protein 1 (PTTG1), APG5 autophagy 5-like (ATG5), CTD-binding SR-like protein rA9 (KIAA1542), Ubiquitin-conjugating enzyme E2L3 (UBE2L3), PX domain containing serine/threonine kinase (PXK), Fc fragment of IgG, low affinity IIa, Receptor (FCGR2A), Tumor Necrosis Factor (ligand) Superfamily 4 (TNFSF4), interleukin-1 receptor-associated kinase 1 (IRAK1), and B-cell scaffold protein with Ankyrin repeats 1 (BANK1). The variant reaching genomewide significance in the meta-analysis was advanced into the analysis (Table 2, Table 3). In the remaining 4 loci, the reported variant or near-perfect proxy (r²>0.75) was not genotyped in our genomewide data set of 1310 SLE cases and 7859 controls (Table 4).

The corrected meta-analysis association statistic was determined by the summing of the Z-scores weighted for cohort size for the current case series and the reports from Kozyrev et al., Nat Genet. 40(2):211-6 (2008), Oishi et al., Journal of Human Genetics 53(2):151-62 (2008), and Sawalha et al., PLoS ONE 3(3):e1727 (2008). The meta-analysis between the current cases series and the association scan described by Harley et al., Nat Genet. 40(2):204-10 (2008) had considerable overlap in the control samples used. The meta-analysis for these alleles was therefore conducted by merging the SLE cases from the Harley et al. report and the current cases series and calculating the association statistic relative to the 7859 controls described above. For the family-based study described by Cunninghame Graham et al., Nat Genet. 40(1):83-89 (2008) the meta-analysis was conducted using Fisher's combined probability test.

TABLE 1
Confirmed SLE risk loci and SLE risk alleles based on presence of two published reports with P ≦ 1 × 10⁻⁵.

| | | | | | Report 2 | | | | | Current cases series* | |
|---|---|---|---|---|---|---|---|---|---|---|---|
| | Chro- | Report 1 | | | | r² to allele | | | Additional | SNP | r² to allele |
| Locus | mosome | Allele | P value | Ref. | Allele | in Report 1 | P value | Ref. | references | (allele) | in Report 1 |
| PTPN22 | 1p13.2 | rs2476601 | 1.0 × 10⁻⁵ | 13 | rs2476601 | 1.00 | 5.2 × 10⁻⁶ | 14 | 26 | rs2476601 | 1.00 |
| | | (SEQ ID | | | (SEQ ID | | | | | (SEQ ID NOS | |
| | | NOS | | | NOS | | | | | 1 and 2) | |
| | | 1 and 2) | | | 1 and 2) | | | | | | |
| STAT4 | 2q32.2 | rs7574865 | 1.9 × 10⁻⁹ | 7 | rs7574865 | 1.00 | 2.8 × 10⁻⁹ | 14 | 1 | rs7574865 | 1.00 |
| | | (SEQ ID | | | (SEQ ID | | | | | (SEQ ID NOS | |
| | | NOS | | | NOS | | | | | 3 and 4) | |
| | | 3 and 4) | | | 3 and 4) | | | | | | |
| HLA-DR2 | 6p21.32 | DRB1*1501 | 1.0 × 10⁻⁵ | 11 | DRB1*1501 | 1.00 | 1.0 × 10⁻⁷ | 12 | 27 | rs3129860 | 0.97 |
| | | | | | | | | | | (SEQ ID NOS | |
| | | | | | | | | | | 15 and 16) | |
| HLA-DR3 | 6p21.32 | DRB1*0301 | 10 × 10⁻⁶ | 11 | DRB1*0301 | 1.00 | 1.0 × 10⁻⁵ | 12 | 1, 14, 27, | rs2187668 | 0.87 |
| | | | | | | | | | 28 | (SEQ ID NOS | |
| | | | | | | | | | | 17 and 18) | |
| IRF5 | 7q32.1 | rs2004640 | 5.2 × 10⁻⁸ | 15 | rs2004640 | 1.00 | 4.4 × 10⁻¹⁶ | 16 | 1, 14, 29, | rs10488631 | — |
| | | (SEQ ID | | | (SEQ ID | | | | 30 | (SEQ ID NOS | |
| | | NOS | | | NOS | | | | | 19 and 20) | |
| | | 5 and 6) | | | 5 and 6) | | | | | | |
| BLK | 8p23.1 | rs13277113 | 10 × 10⁻¹⁰ | 1 | rs6985109 | 0.33 | 2.5 × 10⁻¹¹ | 14 | | rs13277113 | 1.00 |
| | | (SEQ ID | | | (SEQ ID | | | | | (SEQ ID NOS | |
| | | NOS | | | NOS | | | | | 7 and 8) | |
| | | 7 and 8) | | | 11 and 12) | | | | | | |
| ITGAM | 16p11.2 | rs1143679 | 6.9 × 10⁻²² | 17 | rs11574637 | — | 3.0 × 10⁻¹¹ | 1 | 14 | rs9888739 | 0.86 |
| | | (SEQ ID | | | (SEQ ID | | | | | (SEQ ID NOS | |
| | | NOS | | | NOS | | | | | 21 and 22) | |
| | | 9 and 10) | | | 13 and 14) | | | | | | |

*1310 SLE cases and 7859 controls

TABLE 2
Association statistics for 16 confirmed SLE risk loci and 16 confirmed SLE risk alleles in a
genomewide association scan of 1310 SLE cases and 7859 controls. The alleles are ordered by P value.

| | | | | | Allele | | | |
|---|---|---|---|---|---|---|---|---|
| | | SNP | Position* | Minor | frequency | | | Odds ratio |
| Locus | Chromosome | (allele) | (Mb) | allele | Case | Control | P value | (95% CI) |
| HLA-DR3 | 6p21.32 | rs2187668 | 32.714 | T | 0.190 | 0.117 | 9.5 × 10⁻²⁵ | 1.76 (1.58-1.97) |
| | | (SEQ ID NOS | | | | | | |
| | | 17 and 18) | | | | | | |
| IRF5 | 7q32.1 | rs10488631 | 128.188 | C | 0.170 | 0.109 | 1.4 × 10⁻¹⁹ | 1.68 (1.50-1.89) |
| | | (SEQ ID NOS | | | | | | |
| | | 19 and 20) | | | | | | |
| STAT4 | 2q32.2 | rs7574865 | 191.790 | T | 0.312 | 0.235 | 2.5 × 10⁻¹⁴ | 1.48 (1.34-1.64) |
| | | (SEQ ID NOS | | | | | | |
| | | 3 and 4) | | | | | | |
| ITGAM | 16p11.2 | rs9888739 | 31.221 | T | 0.175 | 0.127 | 2.3 × 10⁻¹¹ | 1.46 (1.31-1.63) |
| | | (SEQ ID NOS | | | | | | |
| | | 21 and 22) | | | | | | |
| BLK | 8p23.1 | rs13277113 | 11.387 | A | 0.294 | 0.242 | 1.7 × 10⁻⁸ | 1.30 (1.19-1.43) |
| | | (SEQ ID NOS | | | | | | |
| | | 7 and 8) | | | | | | |
| PTTG1 | 5q33.3 | rs2431697 | 159.813 | C | 0.389 | 0.438 | 3.3 × 10⁻⁶ | 0.82 (0.75-0.89) |
| | | (SEQ ID NOS | | | | | | |
| | | 23 and 24) | | | | | | |
| ATG5 | 6q21 | rs6568431 | 106.695 | A | 0.423 | 0.376 | 5.5 × 10⁻⁶ | 1.22 (1.12-1.32) |
| | | (SEQ ID NOS | | | | | | |
| | | 25 and 26) | | | | | | |
| TNFSF4 | 1q25.1 | rs10489265 | 169.968 | C | 0.278 | 0.238 | 8.7 × 10⁻⁶ | 1.24 (1.09-1.30) |
| | | (SEQ ID NOS | | | | | | |
| | | 27 and 28) | | | | | | |
| PTPN22 | 1p13.2 | rs2476601 | 114.090 | A | 0.116 | 0.089 | 8.9 × 10⁻⁶ | 1.35 (1.18-1.54) |
| | | (SEQ ID NOS | | | | | | |
| | | 1 and 2) | | | | | | |
| IRAK1 | Xq28 | rs2269368 | 152.711 | T | 0.175 | 0.141 | 1.1 × 10⁻⁵ | 1.29 (1.15-1.45) |
| | | (SEQ ID NOS | | | | | | |
| | | 29 and 30) | | | | | | |
| FCGR2A | 1q23.3 | rs1801274 | 158.293 | A | 0.463 | 0.500 | 4.1 × 10⁻⁴ | 0.86 (0.79-0.94) |
| | | (SEQ ID NOS | | | | | | |
| | | 31 and 32) | | | | | | |
| KIAA1542 | 11p15.5 | rs4963128 | 0.580 | T | 0.303 | 0.333 | 3.1 × 10⁻³ | 0.87 (0.80-0.96) |
| | | (SEQ ID NOS | | | | | | |
| | | 33 and 34) | | | | | | |
| UBE2L3 | 22q11.21 | rs5754217 | 20.264 | T | 0.215 | 0.192 | 6.4 × 10⁻³ | 1.15 (1.04-1.27) |
| | | (SEQ ID NOS | | | | | | |
| | | 35 and 36) | | | | | | |
| PXK | 3p14.3 | rs6445975 | 58.345 | G | 0.305 | 0.281 | 0.010 | 1.13 (1.03-1.23) |
| | | (SEQ ID NOS | | | | | | |
| | | 37 and 38) | | | | | | |
| HLA-DR2 | 6p21.32 | rs3129860 | 32.509 | A | 0.160 | 0.147 | 0.092 | 1.10 (0.98-1.24) |
| | | (SEQ ID NOS | | | | | | |
| | | 15 and 16) | | | | | | |
| BANK1 | 4q24 | rs10516487 | 103.108 | A | 0.288 | 0.304 | 0.096 | 0.93 (0.85-1.01) |
| | | (SEQ ID NOS | | | | | | |
| | | 39 and 40) | | | | | | |

*Positions are from NCBI Build 35.

TABLE 3
SLE risk loci and SLE risk alleles with one published report with P ≦ 1 × 10⁻⁵.
Loci with a Meta P ≦ 5 × 10⁻⁸ were considered confirmed and advanced further in the analysis (See Table 2).

| | | Report | | | Current cases series* | | | |
|---|---|---|---|---|---|---|---|---|
| | | SNP | | | SNP | r² to allele | | |
| Locus | Chromosome | (allele) | P value | Reference | (allele) | in Report | P value | Meta P |
| PTTG1 | 5q33.3 | rs2431697 | 1.0 × 10⁻¹⁰ | 14 | rs2431697 | 1.00 | 3.3 × 10⁻⁶ | 5.3 × 10⁻¹⁴ |
| | | (SEQ ID NOS | | | (SEQ ID NOS | | | |
| | | 23 and 24) | | | 23 and 24) | | | |
| ATG5 | 6q21 | rs6568431 | 1.7 × 10⁻⁸ | 14 | rs6568431 | 1.00 | 5.5 × 10⁻⁶ | 2.7 × 10⁻¹² |
| | | (SEQ ID NOS | | | (SEQ ID NOS | | | |
| | | 25 and 26) | | | 25 and 26) | | | |
| IRAK1 | Xq28 | rs2075596 | 2.8 × 10⁻⁷ | 24 | rs2269368 | 0.79 | 1.1 × 10⁻⁵ | 1.4 × 10⁻¹¹ |
| | | (SEQ ID NOS | | | (SEQ ID NOS | | | |
| | | 41 and 42) | | | 29 and 30) | | | |
| TNFSF4 | 1q25.1 | rs12039904 | 4.3 × 10⁻⁷ | 20 | rs10489265 | 0.91 | 8.7 × 10⁻⁶ | 1.0 × 10⁻¹⁰ |
| | | (SEQ ID NOS | | | (SEQ ID NOS | | | |
| | | 43 and 44) | | | 27 and 28) | | | |
| KIAA1542 | 11p15.5 | rs4963128 | 3.0 × 10⁻¹⁰ | 14 | rs4963128 | 1.00 | 3.1 × 10⁻³ | 1.0 × 10⁻⁹ |
| | | (SEQ ID NOS | | | (SEQ ID NOS | | | |
| | | 33 and 34) | | | 33 and 34) | | | |
| UBE2L3 | 22q11.21 | rs5754217 | 7.5 × 10⁻⁸ | 14 | rs5754217 | 1.00 | 6.4 × 10⁻³ | 7.3 × 10⁻⁹ |
| | | (SEQ ID NOS | | | (SEQ ID NOS | | | |
| | | 35 and 36) | | | 35 and 36) | | | |
| BANK1 | 4q24 | rs10516487 | 3.7 × 10⁻¹⁰ | 22 | rs10516487 | 1.00 | 0.096 | 1.0 × 10⁻⁸ |
| | | (SEQ ID NOS | | | (SEQ ID NOS | | | |
| | | 39 and 40) | | | 39 and 40) | | | |
| PXK | 3p14.3 | rs6445975 | 7.1 × 10⁻⁹ | 14 | rs6445975 | 1.00 | 0.010 | 1.0 × 10⁻⁸ |
| | | (SEQ ID NOS | | | (SEQ ID NOS | | | |
| | | 37 and 38) | | | 37 and 38) | | | |
| FCGR2A | 1q23.3 | rs1801274 | 6.8 × 10⁻⁷ | 14 | rs1801274 | 1.00 | 4.1 × 10⁻⁴ | 3.9 × 10⁻⁸ |
| | | (SEQ ID NOS | | | (SEQ ID NOS | | | |
| | | 31 and 32) | | | 31 and 32) | | | |
| NMNAT2 | 1q25.3 | rs2022013 | 1.1 × 10⁻⁷ | 14 | rs2022013 | 1.00 | 0.15 | 5.1 × 10⁻⁶ |
| | | (SEQ ID NOS | | | (SEQ ID NOS | | | |
| | | 45 and 46) | | | 45 and 46) | | | |
| ICA1 | 7p21.3 | rs10156091 | 1.9 × 10⁻⁷ | 14 | rs10156091 | 1.00 | 0.095 | 2.0 × 10⁻⁵ |
| | | (SEQ ID NOS | | | (SEQ ID NOS | | | |
| | | 47 and 48) | | | 47 and 48) | | | |
| LYN | 8q12.1 | rs7829816 | 5.4 × 10⁻⁹ | 14 | rs7829816 | 1.00 | 0.48 | 3.6 × 10⁻³ |
| | | (SEQ ID NOS | | | (SEQ ID NOS | | | |
| | | 49 and 50) | | | 49 and 50) | | | |
| SCUBE1 | 22q13.2 | rs2071725 | 1.2 × 10⁻⁷ | 14 | rs2071725 | 1.00 | 0.63 | 8.3 × 10⁻³ |
| | | (SEQ ID NOS | | | (SEQ ID NOS | | | |
| | | 51 and 52) | | | 51 and 52) | | | |
| ITPR3 | 6p21.31 | rs3748079 | 2.9 × 10⁻⁸ | 23 | rs3748079 | 1.00 | 0.95 | — |
| | | (SEQ ID NOS | | | (SEQ ID NOS | | | |
| | | 53 and 54) | | | 53 and 54) | | | |

*1310 SLE cases and 7859 controls.

TABLE 4
SLE risk loci and SLE risk alleles with one published report with
P ≦ 1 × 10⁻⁵ but lacking a proxy (r² > 0.75) in the 1310 SLE
case/7859 control genomewide association scan. These loci and
alleles are unable to be confirmed with the available data.

| | | Report | | |
|---|---|---|---|---|
| | | SNP | | |
| Locus | Chromosome | (allele) | P value | Reference |
| CRP | 1q23.2 | rs3093061 | 6.4 × 10⁻⁷ | 21 |
| | | (SEQ ID NOS | | |
| | | 55 and 56) | | |
| SELP | 1q24.2 | rs3917815 | 5.7 × 10⁻⁶ | 19 |
| | | (SEQ ID NOS | | |
| | | 57 and 58) | | |
| PDCD1 | 2q37.3 | rs11568821 | 1.0 × 10⁻⁵ | 18 |
| | | (SEQ ID NOS | | |
| | | 59 and 60) | | |
| TYK2 | 19p13.2 | rs2304256 | 2.2 × 10⁻⁸ | 15 |
| | | (SEQ ID NOS | | |
| | | 61 and 62) | | |

### Example 2

### Association of Confirmed SLE Risk Loci and Confirmed SLE Risk Alleles with Autoantibodies to RNA Binding Proteins

### Measurement of Autoantibodies to RNA-Binding Proteins

A total of 1269 serum samples were available from the 1310 SLE cases included in the genomewide association scan. QUANTA Plex ENA Profile 5 Luminex fluorescent immunoassay kits (Inova Diagnostics, San Diego, Calif.) were used to measure IgG autoAbs directed against the RNA-binding proteins SSA (SSA60 and SSA52), SSB, RNP, and Sm in SLE cases from ABCoN, MADGC, and Pittsburgh. QUANTA Plex SLE Profile 8 Luminex fluorescent immunoassay kits (Inova Diagnostics, San Diego, Calif.) were used to measure titers of SSA60, SSA52, SSB, RNP in serum samples from the UCSF SLE cases. Samples positive for autoAbs against either SSA60 or SSA52 were considered SSA positive. SLE cases positive for one or more anti-RBP autoAbs were classified as RBP-pos, and cases lacking anti-RBP autoAbs were classified as RBP-neg.

Serum samples were diluted and run on a Luminex 100 IS system following the manufacturer's protocol. Results were calculated by dividing the median fluorescence intensity (MFI) of the samples by the MFI of the calibrator for each antigen, then multiplying the result by the number of LU (Luminex Units) assigned to the calibrator for that antigen as specified by the manufacturer's protocol. The cutoff values used were: Negative <20 LU; Positive ≧20 LU. Duplicate serum samples were analyzed, and discordant results were resolved by additional testing. The frequency of anti-RBP autoAbs in the SLE cases is presented in Tables 5 and 6.

TABLE 5
Prevalence of autoantibodies to RNA binding proteins (RBPs) in
three independent SLE case series.

| | Case | Case | Case | |
|---|---|---|---|---|
| Autoantibody | Series 1^{†} | Series 2 | Series 3 | All SLE |
| Specificity* | (N = 401) | (N = 572) | (N = 296) | (N = 1269) |
| SSA | 23.2% | 24.5% | 30.1% | 25.4% |
| SSB | 9.5% | 8.9% | 13.9% | 10.2% |
| SSA and/or SSB | 23.7% | 25.0% | 31.4% | 26.1% |
| RNP | 18.5% | 14.0% | 21.6% | 17.2% |
| Sm | 9.5% | 7.7% | 12.8% | 9.5% |
| RNP and/or Sm | 19.7% | 14.9% | 23.3% | 18.4% |
| ≧1 anti-RBP | 37.7% | 35.0% | 45.9% | 38.4% |
| autoantibody | | | | |

*Autoantibodies to SSA, SSB, RNP, and Sm were measured in the serum of 1269 SLE cases using bead-based ELISA assays.
^{†}Case series 1 - Autoimmune Biomarkers Consortium (ABCoN) cases from Johns Hopkins School of Medicine, with additional cases from the Multiple Autoimmune Genetics Consortium (MADGC); case series 2 - University of California, San Francisco; case series 3 - University of Pittsburgh.

TABLE 6
The prevalence of anti-RNA binding protein (anti-RBP)
autoantibodies in three independent SLE case series.

| | Case | | | |
|---|---|---|---|---|
| | series 1 | Case | Case | |
| Number of | (ABCoN + | series 2 | series 3 | |
| anti-RBP | MADGC; | (UCSF; | (Pittsburgh; | All SLE |
| autoAbs | N = 401) | N = 572) | N = 296) | (N = 1269) |
| ≧1 | 37.7% | 35.0% | 45.9% | 38.4% |
| 0 | 62.3% | 65.0% | 54.1% | 61.6% |
| 1 | 18.7% | 18.9% | 22.0% | 19.5% |
| 2 | 15.7% | 12.9% | 17.2% | 14.8% |
| 3 | 2.5% | 2.3% | 5.1% | 3.0% |
| 4 | 0.7% | 0.9% | 1.7% | 1.0% |

The results from these assays were compared to data available from medical records, where available. For the ABCON cohort, concordance between medical records and the INOVA testing was 84% for SSA, 91% for SSB, 85% for RNP and 85% for Sm. For the UCSF cohort, concordance between medical records and the INOVA results was 92% for SSA, 91% for SSB, 91% for RNP and 90% for Sm. Thus, overall there was excellent correlation of these measured anti-RBP autoantibody data with the information available from medical charts. The Luminex technology used here was more sensitive for the detection of anti-RBP autoantibodies than previous methods (Delpech et al., Journal of Clin Lab Analysis 7(4):197-202 (1993), thus the Luminex results were used for all analyses.

Association of Confirmed SLE Risk Loci and Confirmed SLE Risk Alleles with Autoantibodies to RNA Binding Proteins

Each case series was grouped into RBP-pos (SLE cases positive for one or more anti-RBP autoAbs) and RPB-neg (cases lacking anti-RBP autoAbs) subsets and allele frequencies at each of the 16 confirmed SLE risk alleles were determined as follows. Allele frequencies of the 16 confirmed SLE risk alleles were calculated for the 487 SLE cases positive for at least one anti-RNA binding protein autoantibody (SSA, SSB, RNP or Sm), the 782 SLE cases negative for anti-RBP autoantibodies, and the 7859 control samples. Allele frequencies for each case series are shown in Table 7. The SLE risk alleles were tested for significant enrichment in RBP-positive SLE cases vs. the RBP-negative cases using 2×2 contingency tables. In addition to the nominal P value, empiric P values were calculated for each allele by 1 million random permutations of the RBP status of the SLE cases using PLINK (Purcell et al., Am J Hum Genet. 81(3):559-75 (2007) (Table 8). The allele frequencies and association statistics for cases positive for SSA and/or SSB autoAbs, or positive for RNP and/or Sm autoAbs, were calculated and are shown in Table 8.

TABLE 7
Allele frequencies for 16 confirmed SLE risk loci and 16 confirmed SLE risk alleles in RBP-pos cases, RBP-neg cases, and
controls for each of the case series.

| | | | | Case series 2 | | Case series 3 | | | | |
|---|---|---|---|---|---|---|---|---|---|---|
| | | Case series 1 | | UCSF | | Pittsburgh | | All SLE | | |
| | | ABCoN + MADGC | | (N = 572) | | (N = 296) | | (N = 1269) | | |
| | | (N = 401) | | RBP- | RBP- | RBP- | RBP- | RBP- | | |
| | SNP | RBP-pos | | pos | neg | pos | neg | pos | RBP-neg | Controls |
| Locus | (allele) | (N = 151) | RBP-neg (N = 250) | (N = 200) | (N = 372) | (N = 136) | (N = 160) | (N = 487) | (N = 782) | (N = 7859) |
| HLA-DR3 | rs2187668 | 0.235 | 0.144 | 0.264 | 0.144 | 0.257 | 0.172 | 0.253 | 0.150 | 0.117 |
| | (SEQ ID NOS | | | | | | | | | |
| | 17 and 18) | | | | | | | | | |
| HLA-DR2 | rs3129860 | 0.198 | 0.145 | 0.215 | 0.132 | 0.177 | 0.129 | 0.199 | 0.136 | 0.147 |
| | (SEQ ID NOS | | | | | | | | | |
| | 15 and 16) | | | | | | | | | |
| TNFSF4 | rs10489265 | 0.311 | 0.256 | 0.351 | 0.250 | 0.294 | 0.225 | 0.323 | 0.247 | 0.238 |
| | (SEQ ID NOS | | | | | | | | | |
| | 27 and 28) | | | | | | | | | |
| IRAK1 | rs2269368 | 0.183 | 0.120 | 0.225 | 0.179 | 0.200 | 0.134 | 0.205 | 0.151 | 0.141 |
| | (SEQ ID NOS | | | | | | | | | |
| | 29 and 30) | | | | | | | | | |
| STAT4 | rs7574865 | 0.350 | 0.283 | 0.358 | 0.279 | 0.343 | 0.317 | 0.351 | 0.288 | 0.235 |
| | (SEQ ID NOS | | | | | | | | | |
| | 3 and 4) | | | | | | | | | |
| UBE2L3 | rs5754217 | 0.255 | 0.196 | 0.235 | 0.207 | 0.265 | 0.166 | 0.250 | 0.195 | 0.192 |
| | (SEQ ID NOS | | | | | | | | | |
| | 35 and 36) | | | | | | | | | |
| IRF5 | rs10488631 | 0.199 | 0.150 | 0.198 | 0.159 | 0.206 | 0.138 | 0.200 | 0.152 | 0.109 |
| | (SEQ ID NOS | | | | | | | | | |
| | 19 and 20) | | | | | | | | | |
| BANK1 | rs10516487 | 0.255 | 0.300 | 0.250 | 0.290 | 0.302 | 0.309 | 0.266 | 0.297 | 0.304 |
| | (SEQ ID NOS | | | | | | | | | |
| | 39 and 40) | | | | | | | | | |
| KIAA1542 | rs4963128 | 0.268 | 0.329 | 0.302 | 0.312 | 0.282 | 0.286 | 0.286 | 0.312 | 0.333 |
| | (SEQ ID NOS | | | | | | | | | |
| | 33 and 34) | | | | | | | | | |
| ATG5 | rs6568431 | 0.440 | 0.436 | 0.468 | 0.410 | 0.397 | 0.381 | 0.439 | 0.412 | 0.376 |
| | (SEQ ID NOS | | | | | | | | | |
| | 25 and 26) | | | | | | | | | |
| BLK | rs13277113 | 0.305 | 0.288 | 0.305 | 0.273 | 0.313 | 0.316 | 0.307 | 0.286 | 0.242 |
| | SEQ ID NOS | | | | | | | | | |
| | 7 and 8) | | | | | | | | | |
| PTTG1 | rs2431697 | 0.364 | 0.358 | 0.378 | 0.399 | 0.397 | 0.459 | 0.379 | 0.398 | 0.438 |
| | (SEQ ID NOS | | | | | | | | | |
| | 23 and 24) | | | | | | | | | |
| PXK | rs6445975 | 0.331 | 0.280 | 0.310 | 0.319 | 0.302 | 0.284 | 0.314 | 0.299 | 0.281 |
| | (SEQ ID NOS | | | | | | | | | |
| | 37 and 38) | | | | | | | | | |
| PTPN22 | rs2476601 | 0.147 | 0.102 | 0.110 | 0.118 | 0.096 | 0.113 | 0.118 | 0.112 | 0.089 |
| | (SEQ ID NOS | | | | | | | | | |
| | 1 and 2) | | | | | | | | | |
| FCGR2A | rs1801274 | 0.480 | 0.464 | 0.440 | 0.462 | 0.456 | 0.469 | 0.457 | 0.464 | 0.500 |
| | (SEQ ID NOS | | | | | | | | | |
| | 31 and 32) | | | | | | | | | |
| ITGAM | rs9888739 | 0.146 | 0.162 | 0.198 | 0.185 | 0.188 | 0.169 | 0.179 | 0.174 | 0.127 |
| | (SEQ ID NOS | | | | | | | | | |
| | 21 and 22) | | | | | | | | | |

TABLE 8
Allele frequencies and association statistics for 16 confirmed SLE risk loci and 16 confirmed SLE risk alleles in anti-RNA
binding protein (RBP) autoantibody subgroups. The allele frequencies of SLE cases positive for autoAbs to at least one of four RBPs
(SSA, SSB, RNP or Sm), positive for autoAbs to SSA or SSB, and positive for autoAbs to RNP or Sm were compared to SLE cases
negative for the respective autoantibodies. The P values from a permutation analysis randomizing anti-RBP autoAb status are
provided.

| | | | Anti-RBP | | | | | | | | | |
|---|---|---|---|---|---|---|---|---|---|---|---|---|
| | | | (SSA, SSB, RNP or Sm) | | | | | | | | | |
| | | | Allele | | | | Anti-SSA and/or SSB | | | Anti-RNP and/or Sm | | |
| | | | frequency | | | | Allele frequency | | | Allele frequency | | |
| | | Allele | Pos | Neg | | | | Neg | Pos vs. | | Neg | Pos vs. |
| | SNP | frequency | (N = | (N = | Pos vs. neg | | Pos | (N = | neg | Pos | (N = | neg |
| Locus | (allele) | Controls | 487) | 782) | P value | Permuted P | (N = 331) | 938) | P value | (N = 233) | 1036) | P value |
| HLA-DR3 | rs2187668 | 0.117 | 0.253 | 0.150 | 1.2 × 10⁻¹⁰ | <1 × 10⁻⁶ | 0.317 | 0.145 | 2.9 × 10⁻²² | 0.155 | 0.197 | 0.034 |
| | (SEQ ID NOS | | | | | | | | | | | |
| | 17 and 18) | | | | | | | | | | | |
| HLA-DR2 | rs3129860 | 0.147 | 0.199 | 0.136 | 2.4 × 10⁻⁵ | 3.1 × 10⁻⁵ | 0.196 | 0.147 | 3.8 × 10⁻³ | 0.220 | 0.147 | 9.8 × 10⁻⁵ |
| | (SEQ ID NOS | | | | | | | | | | | |
| | 15 and 16) | | | | | | | | | | | |
| TNFSF4 | rs10489265 | 0.238 | 0.323 | 0.247 | 3.3 × 10⁻⁵ | 3.7 × 10⁻⁵ | 0.327 | 0.258 | 5.9 × 10⁻⁴ | 0.310 | 0.268 | 0.066 |
| | (SEQ ID NOS | | | | | | | | | | | |
| | 27 and 28) | | | | | | | | | | | |
| IRAK1 | rs2269368 | 0.141 | 0.205 | 0.151 | 5.9 × 10⁻⁴ | 5.9 × 10⁻⁴ | 0.198 | 0.162 | 0.036 | 0.207 | 0.164 | 0.027 |
| | (SEQ ID NOS | | | | | | | | | | | |
| | 29 and 30) | | | | | | | | | | | |
| STAT4 | rs7574865 | 0.235 | 0.351 | 0.288 | 9.5 × 10⁻⁴ | 1.0 × 10⁻³ | 0.341 | 0.302 | 0.066 | 0.364 | 0.301 | 8.1 × 10⁻³ |
| | (SEQ ID NOS | | | | | | | | | | | |
| | 3 and 4) | | | | | | | | | | | |
| UBE2L3 | rs5754217 | 0.192 | 0.250 | 0.195 | 1.2 × 10⁻³ | 8.5 × 10⁻⁴ | 0.243 | 0.206 | 0.047 | 0.253 | 0.208 | 0.030 |
| | (SEQ ID NOS | | | | | | | | | | | |
| | 35 and 36) | | | | | | | | | | | |
| IRF5 | rs10488631 | 0.109 | 0.200 | 0.152 | 1.6 × 10⁻³ | 1.4 × 10⁻³ | 0.215 | 0.155 | 4.4 × 10⁻⁴ | 0.193 | 0.165 | 0.15 |
| | (SEQ ID NOS | | | | | | | | | | | |
| | 19 and 20) | | | | | | | | | | | |
| BANK1 | rs10516487 | 0.304 | 0.266 | 0.297 | 0.088 | | 0.280 | 0.287 | 0.70 | 0.253 | 0.293 | 0.090 |
| | (SEQ ID NOS | | | | | | | | | | | |
| | 39 and 40) | | | | | | | | | | | |
| KIAA1542 | rs4963128 | 0.333 | 0.286 | 0.312 | 0.16 | | 0.287 | 0.307 | 0.32 | 0.295 | 0.303 | 0.73 |
| | (SEQ ID NOS | | | | | | | | | | | |
| | 33 and 34) | | | | | | | | | | | |
| ATG5 | rs6568431 | 0.376 | 0.439 | 0.412 | 0.18 | | 0.443 | 0.416 | 0.23 | 0.442 | 0.418 | 0.35 |
| | (SEQ ID NOS | | | | | | | | | | | |
| | 25 and 26) | | | | | | | | | | | |
| BLK | rs13277113 | 0.242 | 0.307 | 0.286 | 0.27 | | 0.314 | 0.287 | 0.19 | 0.326 | 0.287 | 0.095 |
| | (SEQ ID NOS | | | | | | | | | | | |
| | 7 and 8) | | | | | | | | | | | |
| PTTG1 | rs2431697 | 0.438 | 0.379 | 0.398 | 0.33 | | 0.378 | 0.396 | 0.42 | 0.369 | 0.396 | 0.29 |
| | (SEQ ID NOS | | | | | | | | | | | |
| | 23 and 24) | | | | | | | | | | | |
| PXK | rs6445975 | 0.281 | 0.314 | 0.299 | 0.43 | | 0.323 | 0.299 | 0.23 | 0.290 | 0.308 | 0.43 |
| | (SEQ ID NOS | | | | | | | | | | | |
| | 37 and 38) | | | | | | | | | | | |
| PTPN22 | rs2476601 | 0.089 | 0.118 | 0.112 | 0.66 | | 0.111 | 0.115 | 0.75 | 0.132 | 0.110 | 0.19 |
| | (SEQ ID NOS | | | | | | | | | | | |
| | 1 and 2) | | | | | | | | | | | |
| FCGR2A | rs1801274 | 0.500 | 0.457 | 0.464 | 0.72 | | 0.455 | 0.464 | 0.69 | 0.451 | 0.464 | 0.61 |
| | (SEQ ID NOS | | | | | | | | | | | |
| | 31 and 32) | | | | | | | | | | | |
| ITGAM | rs9888739 | 0.127 | 0.179 | 0.174 | 0.77 | | 0.189 | 0.171 | 0.31 | 0.163 | 0.179 | 0.42 |
| | (SEQ ID NOS | | | | | | | | | | | |
| | 21 and 22) | | | | | | | | | | | |

TABLE 10
Association of anti-RBP autoAbs with the 11 ACR SLE clinical criteria.

| | | ACR criteria | | | | | | | | | | |
|---|---|---|---|---|---|---|---|---|---|---|---|---|
| | | | | Photo- | Oral | | | Neu- | | | | |
| Autoantibody | | Malar | Discoid | sensitivity | ulcers | Arthritis | Serositis | rologic | Hematologic | Immunologic | Renal | ANA* |
| SSA | Pearson | −0.044 | 0.049 | 0.017 | −0.008 | 0.004 | 0.011 | −0.0622 | 0.11 | 0.076 | 0.029 | 0.041 |
| | correlation | | | | | | | | | | | |
| | coefficient | | | | | | | | | | | |
| | P | 0.106 | 0.076 | 0.529 | 0.757 | 0.868 | 0.672 | 0.0231 | <0.001 | 0.004 | 0.276 | 0.129 |
| SSB | Pearson | −0.011 | 0.013 | 0.06 | −0.012 | 0 | 0.012 | −0.0388 | 0.056 | 0.01 | 0.002 | 0.054 |
| | correlation | | | | | | | | | | | |
| | coefficient | | | | | | | | | | | |
| | P | 0.69 | 0.629 | 0.028 | 0.646 | 0.996 | 0.637 | 0.1567 | 0.04 | 0.713 | 0.933 | 0.046 |
| Sm | Pearson | 0.055 | 0.018 | −0.015 | 0.024 | 0.051 | 0.072 | −0.0022 | 0.122 | 0.174 | 0.106 | 0.025 |
| | correlation | | | | | | | | | | | |
| | coefficient | | | | | | | | | | | |
| | P | 0.045 | 0.502 | 0.572 | 0.379 | 0.061 | 0.008 | 0.9359 | <0.001 | <0.001 | <0.001 | 0.357 |
| RNP | Pearson | 0.046 | 0.027 | 0.029 | 0.019 | 0.075 | 0.031 | −0.017 | 0.129 | 0.15 | 0.099 | 0.078 |
| | correlation | | | | | | | | | | | |
| | coefficient | | | | | | | | | | | |
| | P | 0.092 | 0.319 | 0.289 | 0.477 | 0.005 | 0.248 | 0.5348 | <0.001 | <0.001 | <0.001 | 0.004 |
| SSA, SSB, RNP | Pearson | −0.009 | 0.0756 | 0.0208 | −0.0019 | 0.0591 | 0.0375 | −0.0602 | 0.1433 | 0.1369 | 0.0926 | 0.1046 |
| or Sm | correlation | | | | | | | | | | | |
| | coefficient | | | | | | | | | | | |
| | P | 0.7458 | 0.0064 | 0.4479 | 0.9445 | 0.0307 | 0.1717 | 0.0278 | <0.001 | <0.001 | 0.0007 | 0.0001 |

*Anti-Nuclear Autoantibodies.

We assessed the probability of observing 5 of 14 alleles significantly enriched in RBP-pos SLE cases as compared to RBP-neg SLE cases. (While 7 of 16 alleles were enriched, the 2 HLA alleles were previously reported to be associated with anti-RBP autoAbs, so were excluded from the present analysis.) The probability of observing 5 of 14 alleles at their observed P values is (πPᵢ)×(14 choose 5)=7.1×10⁻¹⁴, in which Pᵢ is the observed P value for each of the 5 alleles and “14 choose 5” is the number of unordered combinations of 5 of 14 alleles.

As discussed above, of the 25 loci examined in this study, a total of 16 met our criteria for confirmed SLE risk loci. At each of those 16 loci, we identified one allele that met our criteria for confirmed SLE risk alleles. These are listed in Table 2. By our methodology's definition, all 16 loci and all 16 alleles had individually been identified previously as SLE risk loci or SLE risk alleles, respectively. However, previous reports for three of the loci either showed inconsistent evidence for association across several cohorts or failed to reach a genomewide level of statistical significance (P≦5×10⁻⁸). Those three loci are PTTG1, ATG5, and UBE2L3. Our results now show that they are, according to the methodology described here, confirmed SLE risk loci.

Anti-RBP autoAbs aggregate in SLE-prone families and are found at low frequency in clinically unaffected family members, suggesting a genetic basis for this phenotype (Ramos et al., Genes Immun 7(5):417-32 (2006)). The HLA Class II alleles DR3 (DRB1*0301) and DR2 (DRB1*1501) were initially identified as SLE risk alleles by their enrichment in cases and were subsequently found to be more strongly associated with specific anti-RBP autoAbs than with the global SLE phenotype (reviewed in Harley et al., Curr Opin Immunol 10(6):690-96 (1998)). We therefore tested, as described below, whether the 16 confirmed SLE risk alleles were preferentially associated with anti-RBP autoAbs across the three case series.

The sera of 1,269 SLE cases were tested for anti-RBP autoAbs. Overall, 26.1% of cases were positive for anti-SSA and/or anti-SSB autoAbs, and 18.4% were positive for anti-RNP and/or anti-Sm autoAbs (Table 5). In total, 38.4% of cases were positive for one or more anti-RBP autoAbs. The frequency of anti-RBP autoAbs was higher in case series 3 (P=0.0065); however, a Breslow-Day test of heterogeneity between the three series was not significant for any of the 16 alleles studied, and no significant population stratification was observed for the 1310 cases and 7859 controls (uncorrected λ_{gc}=1.06).

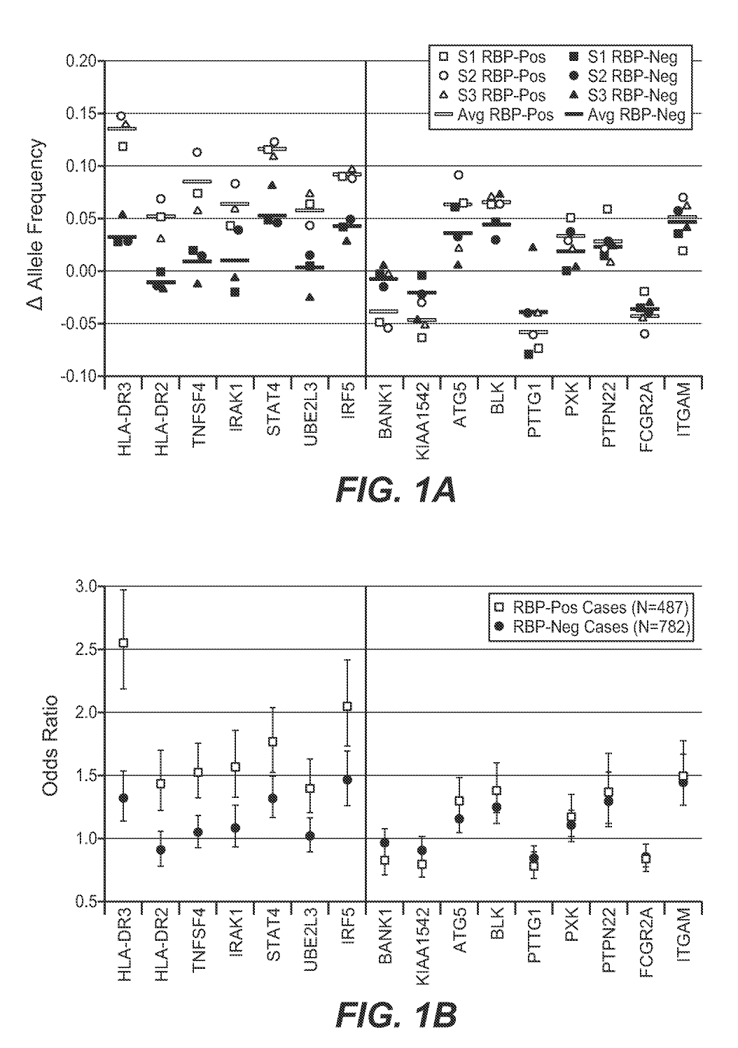

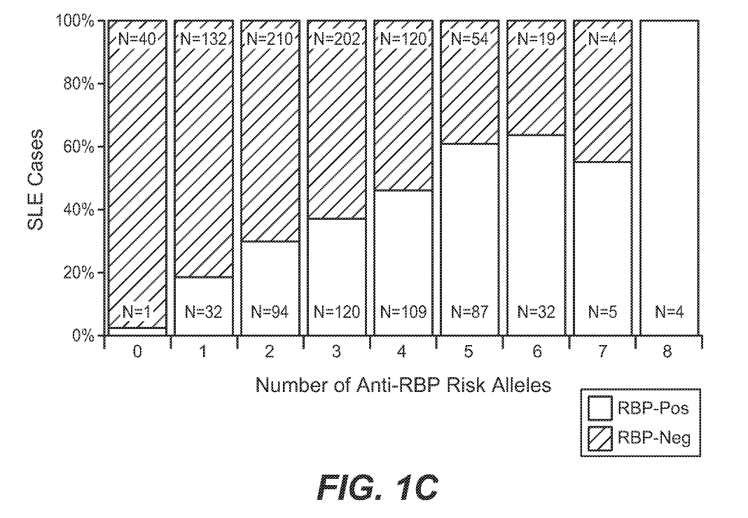

The frequency of 16 confirmed SLE risk alleles was compared in 487 cases positive (RBP-pos) for at least one anti-RBP autoAb (SSA, SSB, RNP, or Sm), 782 cases negative (RBP-neg) for anti-RBP autoAbs, and 7859 controls. The data is presented in Table 9. Allele frequencies differed between the RBP-pos and RBP-neg subset at 7 of the loci: HLA-DR3, P=1.2×10⁻¹⁰; HLA-DR2, P=2.4×10⁻⁵; TNFSF4, P=3.3×10⁻⁵; IRAK1, P=5.9×10⁻⁴; STAT4, P=9.5×10⁻⁴; UBE2L3, P=1.2×10⁻³; and IRF5, P=1.6×10⁻³ (FIG. 1A and Table 9). Given the similar allele frequency trends in each of the three case series, we combined the case series into a single sample (RBP-pos, N=487; RBP-neg, N=782) for subsequent analyses. The odds ratios for association in the combined RBP-pos and RBP-neg subsets are shown in FIG. 1B. Of interest, 4 of the 7 anti-RBP associated loci—HLA-DR2, TNFSF4, IRAK1 and UBE2L3—showed no significant differences in allele frequency between the RBP-neg subset and controls. For the remaining 9 confirmed SLE risk loci—BANK1, K1AA1542, BLK, PTTG1, PXK, PTPN22, FCGR2A, ATG5, and ITGAM—allele frequencies between the RBP-pos and RBP-neg subsets were not significantly different (FIGS. 1A and 1B and Table 9). We conclude that 7 of the 16 genetic loci identified initially by their association with the global SLE phenotype show strong association with the RBP-pos subset of SLE, and lower levels or no association with the RBP-neg subset. These loci are referred to as anti-RBP-associated SLE risk loci. Alleles for each of those anti-RBP-associated SLE risk loci, as identified herein, are referred to as anti-RBP-associated risk alleles.

We next asked whether the number of anti-RBP-associated SLE risk alleles correlated with the presence of anti-RBP autoAbs in serum (FIG. 1C). For the 41 subjects carrying no such risk alleles, only one exhibited anti-RBP autoAbs. For the remaining cases, the overall risk for anti-RBP autoAbs increased with the number of anti-RBP-associated SLE risk alleles in a graded fashion (FIG. 1C), with the odds of having anti-RBP autoAbs increasing by 50% (95% CI=36-66%) for each additional anti-RBP-associated SLE risk allele. The probability of the observed distribution is P<5.2×10⁻²¹.

TABLE 9
Anti-RBP-associated SLE risk loci and anti-RBP-associated SLE risk alleles. A
subset of 7 confirmed SLE risk loci and 7 confirmed SLE risk alleles are associated with
autoantibodies to RNA binding proteins (bold type).

| | | RBP-pos | RBP-neg | | RBP-pos ν | | |
|---|---|---|---|---|---|---|---|
| | | SLE | SLE | Controls | RBP-neg | RBP-pos SLE ν | RBP-neg SLE ν |
| | SNP | (N = 487) | (N = 782) | (N = 7859) | SLE | Controls | Controls |
| Locus | (allele) | Allele Frequencies | | | P | P | P |
| HLA-DR3 | rs2187668 | 0.253 | 0.150 | 0.117 | 1.2 × 10⁻¹⁰ | 3.6 × 10⁻³⁵ | 1.8 × 10⁻⁴ |
| | (SEQ ID NOS | | | | | | |
| | 17 and 18) | | | | | | |
| HL4-DR2 | rs3129860 | 0.199 | 0.136 | 0.147 | 2.4 × 10⁻⁵ | 1.0 × 10⁻⁵ | 0.23 |
| | (SEQ ID NOS | | | | | | |
| | 15 and 16) | | | | | | |
| TNFSF4 | rs10489265 | 0.323 | 0.247 | 0.238 | 3.3 × 10⁻⁵ | 2.0 × 10⁻⁹ | 0.41 |
| | (SEQ ID NOS | | | | | | |
| | 27 and 28) | | | | | | |
| IRAK1 | rs2269368 | 0.205 | 0.151 | 0.141 | 5.9 × 10⁻⁴ | 7.6 × 10⁻⁸ | 0.29 |
| | (SEQ ID NOS | | | | | | |
| | 29 and 30) | | | | | | |
| STAT4 | rs7574865 | 0.351 | 0.288 | 0.235 | 9.5 × 10⁻⁴ | 9.0 × 10⁻¹⁵ | 1.1 × 10⁻⁵ |
| | (SEQ ID NOS | | | | | | |
| | 3 and 4) | | | | | | |
| UBE2L3 | rs5754217 | 0.250 | 0.195 | 0.192 | 1.2 × 10⁻³ | 1.0 × 10⁻⁵ | 0.75 |
| | (SEQ ID NOS | | | | | | |
| | 35 and 36) | | | | | | |
| IRF5 | rs10488631 | 0.200 | 0.152 | 0.109 | 1.6 × 10⁻³ | 3.1 × 10⁻¹⁸ | 3.1 × 10⁻⁷ |
| | (SEQ ID NOS | | | | | | |
| | 19 and 20) | | | | | | |
| BANK1 | rs10516487 | 0.266 | 0.297 | 0.304 | 0.088 | 0.011 | 0.57 |
| | (SEQ ID NOS | | | | | | |
| | 39 and 40) | | | | | | |
| KIAA1542 | rs4963128 | 0.286 | 0.312 | 0.333 | 0.17 | 2.7 × 10⁻³ | 0.098 |
| | (SEQ ID NOS | | | | | | |
| | 33 and 34) | | | | | | |
| ATG5 | rs6568431 | 0.439 | 0.412 | 0.376 | 0.18 | 7.4 × 10⁻⁵ | 4.6 × 10⁻³ |
| | (SEQ ID NOS | | | | | | |
| | 25 and 26) | | | | | | |
| BLK | rs13277113 | 0.307 | 0.286 | 0.242 | 0.27 | 4.8 × 10⁻⁶ | 9.9 × 10⁻⁵ |
| | (SEQ ID NOS | | | | | | |
| | 7 and 8) | | | | | | |
| PTTG1 | rs2431697 | 0.379 | 0.398 | 0.438 | 0.33 | 3.1 × 10⁻⁴ | 2.6 × 10⁻³ |
| | (SEQ ID NOS | | | | | | |
| | 23 and 24) | | | | | | |
| PXK | rs6445975 | 0.314 | 0.299 | 0.281 | 0.43 | 0.025 | 0.12 |
| | (SEQ ID NOS | | | | | | |
| | 37 and 38) | | | | | | |
| PTPN22 | rs2476601 | 0.118 | 0.112 | 0.089 | 0.67 | 2.3 × 10⁻³ | 2.3 × 10⁻³ |
| | (SEQ ID NOS | | | | | | |
| | 1 and 2) | | | | | | |
| FCGR2A | rs1801274 | 0.457 | 0.464 | 0.500 | 0.72 | 8.6 × 10⁻³ | 6.5 × 10⁻³ |
| | (SEQ ID NOS | | | | | | |
| | 31 and 32) | | | | | | |
| ITGAM | rs9888739 | 0.179 | 0.174 | 0.127 | 0.77 | 3.2 × 10⁻⁶ | 1.3 × 10⁻⁷ |
| | (SEQ ID NOS | | | | | | |
| | 21 and 22) | | | | | | |

*Nominal P values for allele frequency differences between RBP-pos and RBP-neg SLE cases were calculated; permutation analysis showed essentially identical statistical significance (See Table 8).
^{†}The HLA-DR3 (DRB1*0301) and DR2 (DRB1*1501) alleles have an r² of 0.87 and 0.97, respectively, to the indicated SNP.

### Example 3

### Association of Confirmed SLE Risk Loci and Confirmed SLE Risk Alleles with Clinical and Pathophysiological Indicators

Association of Anti-RBP Autoabs with Clinical Features ACR Criteria

The presence of the anti-RBP autoAbs (SSA, SSB, RNP and Sm) measured in serum as described above in 1269 SLE cases was examined for a correlation with the 11 ACR clinical criteria (Hochberg et al., Arthritis Rheum 40(9):1725 (1997) (Table 10). The anti-RBPs were significantly associated with the Hematologic, Immunologic and Anti-Nuclear Antibody (ANA) clinical criteria (Hochberg et al., Arthritis Rheum 40(9):1725 (1997). In addition, RNP and Sm were associated with renal involvement. However, when the 7 anti-RBP-associated SLE risk loci were tested in a linear regression model that incorporated sex and recruitment center, no robust associations of the anti-RBP-associated SLE risk loci were observed with the ACR clinical criteria.

### Age at Diagnosis

The 7 anti-RBP-associated SLE risk alleles were tested in a linear regression model that incorporated sex and recruitment center. In this test, an association of the anti-RBP-associated SLE risk alleles with the age at diagnosis was observed. The overall risk for anti-RBP Abs increased with the number of anti-RBP-associated SLE risk alleles in a graded fashion, as discussed above, with the odds of having anti-RBP autoAbs increasing by 50% (95% CI=36-66%) for each additional anti-RBP-associated SLE risk allele. Individuals with 6 anti-RBP-associated SLE risk alleles were, on average, 32.4 years old at diagnosis, while those with 0 anti-RBP-associated SLE risk alleles were, on average, 37.0 years old. Overall, the mean age of diagnosis decreased by 0.72 years (95% CI=0.23-1.21 years, P=0.004) for each additional anti-RBP-associated SLE risk allele (Table 11). These data suggest a dosage effect of anti-RBP-associated SLE risk loci (or alleles) on the anti-RBP autoAb subphenotype and on age of diagnosis of disease.

TABLE 11
Average age at diagnosis in SLE cases stratified by the
number of anti-RBP-associated SLE risk alleles in a linear
regression model that incorporates sex and recruitment center.

| Number | | | |
|---|---|---|---|
| of anti- | Number | | |
| RBP risk | of SLE | Average age | Standard |
| alleles | cases | at diagnosis | deviation |
| 0 | 41 | 36.95 | 1.03 |
| 1 | 165 | 36.32 | 1.14 |
| 2 | 310 | 35.63 | 1.15 |
| 3 | 328 | 34.85 | 1.04 |
| 4 | 233 | 34.15 | 1.10 |
| 5 | 143 | 33.37 | 0.90 |
| 6 | 50 | 32.82 | 1.24 |
| 7 | 10 | 32.74 | 1.69 |
| 8 | 4 | 30.91 | 0.19 |

In certain instances, the type I interferon (IFN) pathway has been implicated in disease pathogenesis. Therefore, we examined a subset of cases to determine whether the anti-RBP-associated SLE risk alleles were correlated with levels of type I IFN regulated gene expression in blood. Gene expression for 274 ABCoN SLE cases and 23 healthy controls was measured in whole blood (PAXgene) RNA using lumina HumanWG-6v2 BeadChips. Raw expression data was normalized in BeadStudio (Illumina) using quantile normalization. An interferon (IFN) signature consisting of 82 IFN-regulated genes was previously identified in an Affymetrix dataset (81 SLE and 42 healthy controls) (Baechler et al., Proc Natl Acad Sci USA 100(5):2610-15 (2003)). Of these 82 genes, 73 genes were measured on the Illumina BeadChip. The expression data for these 73 genes were normalized so that each gene had a maximum value of 1.0. The normalized values of these 73 genes were summed to obtain the IFN gene expression score for each patient. We grouped the SLE cases by the number of anti-RBP-associated SLE risk alleles in each case. The mean IFN gene expression score was then calculated for each group. The significance of the difference in IFN gene expression score distributions between SLE cases with varying numbers of anti-RBP-associated SLE risk alleles was determined by a Student's T-test using a 2 tailed P-value distribution and unequal sample variance.

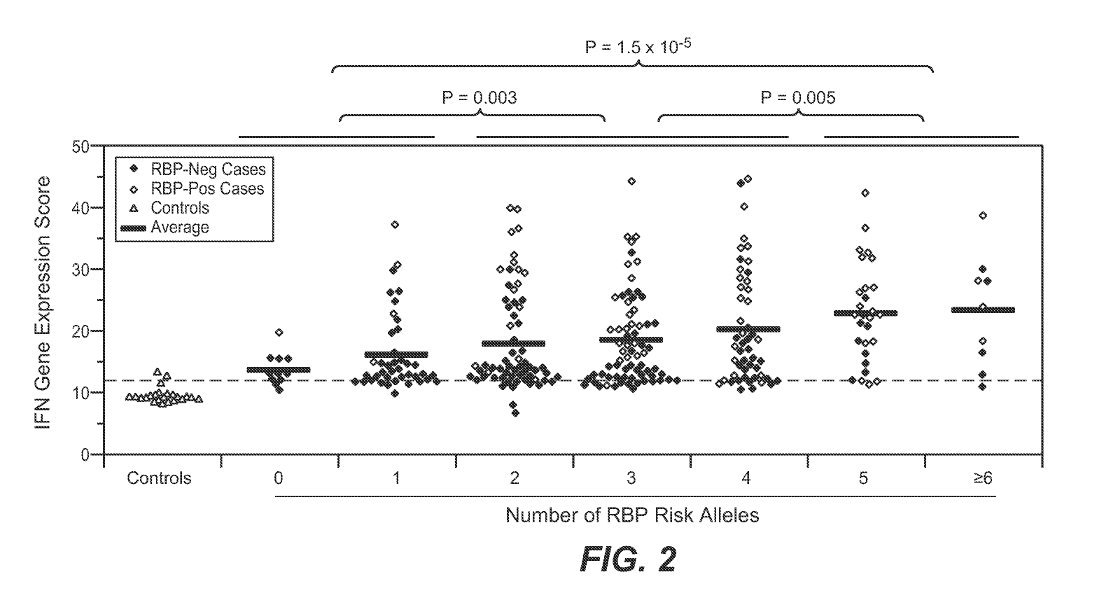

As shown in FIG. 2, SLE cases had elevated levels of IFN inducible gene expression as compared to controls, consistent with previously described results (Baechler et al., Proc Natl Acad Sci USA 100(5):2610-15 (2003); Kirou et al., Arthritis Rheum 50(12):3958-67 (2004)). FIG. 2 also shows that individuals carrying 2, 3 or 4 anti-RBP-associated SLE risk alleles showed, on average, significantly higher IFN gene expression scores than individuals carrying either 0 or 1 anti-RBP-associated SLE risk alleles. Cases with 5 or more risk alleles showed even higher average IFN gene expression scores (FIG. 2). The IFN gene expression score was also strongly associated with the presence of anti-RBP autoAbs (Niewold et al., Genes Immun 8(6):492-502 (2007)). We conclude that anti-RBP autoAb risk alleles are significantly associated in a dose-dependent manner with activation of the type I IFN pathway as measured by IFN-regulated gene expression in blood.

### Example 4

### Genome-Wide Association Scan for Variants Associated with SLE Cases Positive for Antibodies to RNA-Binding Proteins

### Samples and Methodology

Autoantibodies to the RNA-binding proteins SSA, SSB, RNP and Sm were measured as described above in Example 2 in the serum of (i) 1269 SLE cases used in a genome-wide association scan (see Example 2 above); (ii) 342 independent SLE case from the United States (U.S.) (see Gateva et al., Nature Genetics, manuscript accepted for publication, 2009); and (iii) 748 SLE cases collected in Sweden (SWE) (see Gateva et al., Nature Genetics, manuscript accepted for publication, 2009). Genotype data for the 1269 SLE cases from the genome-wide association scan was examined by comparing the allele frequency of the 487 RBP-positive (RBP+) SLE cases (see Example 2 above) to the frequency in the 782 RBP-negative (RBP−) cases. Variants with a P<0.001 for the RBP+ as compared to the RBP− cases were advanced into a replication dataset from the U.S. and Sweden. Genotypes in the replication dataset were measured using a custom Illumina 12K bead array (see Gateva et al., Nature Genetics, manuscript accepted for publication, 2009). The frequency of the variants were measured in the RBP+ and RBP− cases of the replication dataset from U.S. and Sweden by labeling RBP+samples as cases and RBP− samples as controls. Case-control analysis was performed using PLINK (Purcell et al., Am J Hum Genet. 81(3):559-75 (2007)) and one degree of freedom allelic test for association was performed. Meta-analysis combining the three data sets was carried out using the freely available METAL software package (available at the URL www(dot)sph(dot)umich(dot)edu(slash)csg(slash)abecasis(slash)Metal) and total sample sizes were used for weights. Nineteen variants were identified that had a significant P value (P<0.05) in the replication samples. These are shown in Table 12.

### Discussion

An emerging story in human SLE is the important role of the type I interferon (IFN) pathway in disease pathogenesis. Type I IFN is present in serum of SLE cases and can induce macrophages to differentiate into dendritic cells (Blanco et al., Science 294(5546):1540-43 (2001)). The production of IFN is linked to the presence of Ab and nucleic acid containing immune complexes (reviewed in Ronnblom et al., Arthritis Rheum 54(2):408-20 (2006)). The majority of SLE cases exhibit a prominent type I IFN gene expression ‘signature’ in blood cells (Baechler et al., Proc Natl Acad Sci USA 100(5):2610-15 (2003)) and have elevated levels of IFN-inducible cytokines and chemokines in serum (Bauer et al., PLoS medicine 3(12):e491 (2006)). Immune complexes containing native DNA and RNA stimulate toll-like receptors (TLRs) 7 and 9 expressed by dendritic cells and B cells to produce type I IFN which further stimulates immune complex formation (reviewed in Marshak-Rothstein et al., Annu Rev Immunol 25:419-41 (2007)).

Strikingly, all of the anti-RBP-associated SLE risk loci identified in this study have known roles in biochemical and immunologic events initiated by TLR7 and TLR9 signaling. IRF5 is a transcription factor that mediates signaling downstream of TLR7/9 and is important for transactivation of type I IFN and other cytokines (Takaoka et al., Nature 434(7030):243-9 (2005)). The IRF5 risk haplotype drives elevated expression of unique IRF5 protein isoforms and is hypothesized to enhance IFN signaling downstream of TLRs (Graham et al., Proc Natl Acad Sci USA 104(16):6758-63 (2007)). The tyrosine kinase TRAM mediates signaling downstream of TLR4, 7 and 9, and is required for the production of TLR7/9-induced IFN-alpha. Class II antigen-presenting HLA-DR alleles are expressed on the surface of macrophages, dendritic cells and B cells, and are upregulated by TLR7/9 signaling. TNFSF4 (OX40L) is also upregulated following TLR9 ligation and is a potent co-stimulator of CD4+ TH2 T cells that drive autoAb production (Liu et al., J Clin Invest 118(3):1165-75 (2008)). The SLE risk allele for TNFSF4 is associated with prolonged and enhanced TNFSF4 protein expression following B cell stimulation (Cunninghame Graham et al., Nature Genet. 40(1):83-89 (2008)). STAT4 has a role in Ti helper T cell differentiation and, in addition, mediates type I IFN receptor signaling in human T cells and natural killer cells (Miyagi et al., J Exp Med 204(10):2383-96 (2007)). UBE2L3 (also called UbCH₇) is an E2 ubiquitin-conjugating enzyme (Moynihan et al., Mamm Genome;7(7):520-5 (1996)) with many targets, notably TRAF6, a protein that activates IRF5 and is required for the induction of type I IFN following TLR ligation (Takaoka et al., Nature 434(7030):243-9 (2005)). SSA/Ro itself is an IFN-inducible E3 ubiquitin ligase that is ubiquitinated by UBE2L3 (Espinosa et al., J Immunol 176(10):6277-85 (2006)). Thus, the various anti-RBP associated alleles identified here all map to TLR7/9 signaling and downstream immunologic pathways.

In summary, we have confirmed 16 SLE risk loci and 16 SLE risk alleles that are associated with the global SLE phenotype. Significantly, we have further determined that 7 of those SLE risk loci and SLE risk alleles contribute to the anti-RBP autoAb subphenotype of SLE and are referred to anti-RBP-associated SLE risk loci and anti-RBP-associated SLE risk alleles. The known functions of these anti-RBP-associated SLE risk loci suggest a discrete genetic pathway contributing to induction of type I IFN and production of anti-RBP autoAbs. Our results indicate that anti-RBP-associated genetic markers, including the anti-RBP-associated SLE risk loci and anti-RBP-associated SLE risk alleles described herein, may ultimately be useful in objectively identifying the presence of and/or classifying the disease in a patient, in identifying subpopulations of lupus patients, including patients having the anti-RBP autoAb subphenotype, as well as in defining pathophysiological aspects of lupus, clinical activity, response to therapy, and/or prognosis.

### REFERENCES

1. Horn G, Graham R R, Modrek B, et al. Association of systemic lupus erythematosus with C8orf13-BLK and ITGAM-ITGAX. N Engl J Med 2008; 358(9):900-9.
2. Hochberg M C. Updating the American College of Rheumatology revised criteria for the classification of systemic lupus erythematosus. Arthritis Rheum 1997; 40(9):1725.
3. Seligman V A, Suarez C, Lum R, et al. The Fcgamma receptor DIA-158F allele is a major risk factor for the development of lupus nephritis among Caucasians but not non-Caucasians. Arthritis Rheum 2001; 44(3):618-25.
4. Criswell L A, Pfeiffer K A, Lum R F, et al. Analysis of families in the multiple autoimmune disease genetics consortium (MADGC) collection: the PTPN22 620W allele associates with multiple autoimmune phenotypes. Am J Hum Genet. 2005; 76(4):561-71.
5. Bauer J W, Baechler E C, Petri M, et al. Elevated serum levels of interferon-regulated chemokines are biomarkers for active human systemic lupus erythematosus. PLoS medicine 2006; 3(12):e491.
6. Demirci F Y, Manzi S, Ramsey-Goldman R, et al. Association of a common interferon regulatory factor 5 (IRF5) variant with increased risk of systemic lupus erythematosus (SLE). Ann Hum Genet. 2007; 71(Pt 3):308-11.
7. Remmers E F, Plenge R M, Lee A T, et al. STAT4 and the risk of rheumatoid arthritis and systemic lupus erythematosus. N Engl J Med 2007; 357(10):977-86.
8. Purcell S, Neale B, Todd-Brown K, et al. PLINK: a tool set for whole-genome association and population-based linkage analyses. Am J Hum Genet. 2007; 81(3):559-75.
9. Price A L, Patterson N J, Plenge R M, Weinblatt M E, Shadick N A, Reich D. Principal components analysis corrects for stratification in genome-wide association studies. Nat Genet. 2006; 38(8):904-09.
10. Devlin B, Roeder K, Wasserman L. Genomic control for association studies: a semiparametric test to detect excess-haplotype sharing. Biostatistics 2000; 1(4):369-87.
11. Hartung K, Baur, M. P., Coldewey, R., Fricke, M., Kalden, J. R., Lakomek, H. J., Peter, H. H., Schendel, D., Schneider, P. M., Seuchter, S. A., Stangel, W., Deicher, H. R. G. Major histocompatibility complex haplotypes and complement C4 alleles in systemic lupus erythematosus. J Clin Invest 1992; 90:1346-51.
12. Yao Z, Hartung K, Deicher H G, et al. DNA typing for HLA-DPB1-alleles in German patients with systemic lupus erythematosus using the polymerase chain reaction and DIG-ddUTP-labelled oligonucleotide probes. Members of SLE Study Group. Eur J Immunogenet 1993; 20(4):259-66.
13. Lee Y H, Rho Y H, Choi S J, et al. The PTPN22 C1858T functional polymorphism and autoimmune diseases—a meta-analysis. Rheumatology (Oxford, England) 2007; 46(1):49-56.
14. Harley J B, Alarcon-Riquelme M E, Criswell L A, et al. Genome-wide association scan in women with systemic lupus erythematosus identifies susceptibility variants in ITGAM, PXK, KIAA1542 and other loci. Nat Genet. 2008; 40(2):204-10.
15. Sigurdsson S, Nordmark G, Goring H H, et al. Polymorphisms in the tyrosine kinase 2 and interferon regulatory factor 5 genes are associated with systemic lupus erythematosus. Am J Hum Genet. 2005; 76(3):528-37.
16. Graham R R, Kozyrev S V, Baechler E C, et al. A common haplotype of interferon regulatory factor 5 (IRF5) regulates splicing and expression and is associated with increased risk of systemic lupus erythematosus. Nat Genet. 2006; 38(5):550-55.
17. Nath S K, Han S, Kim-Howard X, et al. A nonsynonymous functional variant in integrin-alpha(M) (encoded by ITGAM) is associated with systemic lupus erythematosus. Nat Genet. 2008; 40(2):152-4.
18. Prokunina L, Castillejo-Lopez C, Oberg F, et al. A regulatory polymorphism in PDCD 1 is associated with susceptibility to systemic lupus erythematosus in humans. Nat Genet. 2002; 32(4):666-9.
19. Jacob C O, Reiff A, Armstrong D L, et al. Identification of novel susceptibility genes in childhood-onset systemic lupus erythematosus using a uniquely designed candidate gene pathway platform. Arthritis Rheum 2007; 56(12):4164-73.
20. Cunninghame Graham D S, Graham R R, Manku H, et al. Polymorphism at the TNF Superfamily Gene OX40L Confers Susceptibility to Systemic Lupus Erythematosus. Nature Genetics 2008; 40(1):83-89.
21. Edberg J C, Wu J, Langefeld C D, et al. Genetic variation in the CRP promoter: association with systemic lupus erythematosus. Hum Mol Genet. 2008; 17(8):1147-55.
22. Kozyrev S V, Abelson A K, Wojcik J, et al. Functional variants in the B-cell gene BANK1 are associated with systemic lupus erythematosus. Nat Genet. 2008; 40(2):211-6.
23. Oishi T, Lida A, Otsubo S, et al. A functional SNP in the NKX2.5-binding site of ITPR3 promoter is associated with susceptibility to systemic lupus erythematosus in Japanese population. Journal of human genetics 2008; 53(2):151-62.
24. Sawalha A H, Webb R, Han S, et al. Common variants within MECP2 confer risk of systemic lupus erythematosus. PLoS ONE 2008; 3(3):e1727.
25. Delpech A, Gilbert D, Daliphard S, Le Loet X, Godin M, Tron F. Antibodies to Sm, RNP and SSB detected by solid-phase ELISAs using recombinant antigens: a comparison study with counter immunoelectrophoresis and immunoblotting. Journal of clinical laboratory analysis 1993; 7(4):197-202.
26. Kyogoku C, Langefeld C D, Ortmann W A, et al. Genetic association of the R620W polymorphism of protein tyrosine phosphatase PTPN22 with human SLE. Am J Hum Genet. 2004; 75(3):504-7.
27. Harley J B, Moser K L, Gaffney P M, Behrens T W. The genetics of human systemic lupus erythematosus. Curr Opin Immunol 1998; 10(6):690-96.
28. Fernando M M, Stevens C R, Sabeti P C, et al. Identification of two independent risk factors for lupus within the MHC in United Kingdom families. PLoS Genet. 2007; 3(11):e192.
29. Graham R R, Kyogoku C, Sigurdsson S, et al. Three functional variants of IFN regulatory factor 5 (IRF5) define risk and protective haplotypes for human lupus. Proc Natl Acad Sci USA 2007; 104(16):6758-63.
30. Sigurdsson S, Goring H H, Kristjansdottir G, et al. Comprehensive evaluation of the genetic variants of interferon regulatory factor 5 (IRF5) reveals a novel 5 bp length polymorphism as strong risk factor for systemic lupus erythematosus. Hum Mol Genet. 2008; 17(6):872-81.

TABLE 12
SLE-associated loci and SLE-associated alleles.
Variants associated with RBP + SLE cases compared to RBP− SLE cases in three independent datasets.
All variants displayed a significant replication P value (<0.05) in the US and Swedish samples; n.a. = not available.

| | | | | | | | | US Replication Sample | | | |
|---|---|---|---|---|---|---|---|---|---|---|---|
| | | | | GWAS | | | | (157 RBP+ and 185 RBP− SLE cases) | | | |
| | | | | (487 RBP+ and 782 RBP− SLE cases) | | | | | Allele | Allele | |
| | | | | | Allele | Allele | | | fre- | fre- | |
| | | | | | frequency | frequency | | | quency | quency | |
| SNP | Chr. | Position | Locus | Allele | RBP+ | RBP− | P value | Allele | RBP+ | RBP− | P value |
| rs1005715 | 16 | 73071533 | GLG1 | T | 0.821 | 0.851 | 0.0427134 | C | 0.210 | 0.151 | 0.04516 |
| (SEQ ID NO: 63) | | | (SEQ ID NO: 64) | | | | | | | | |
| rs4838288 | 9 | 127510162 | MAPKAP1 | T | 0.108 | 0.074 | 0.0026669 | T | 0.086 | 0.038 | 0.008204 |
| (SEQ ID NO: 65) | | | (SEQ ID NO: 66) | | | | | | | | |
| rs1419617 | 7 | 125076596 | LOC646841 | T | 0.166 | 0.141 | 0.0418452 | T | 0.124 | 0.089 | 0.137 |
| (SEQ ID NO: 67) | | | (SEQ ID NO: 68) | | | | | | | | |
| rs7775840 | 6 | 146942309 | C6orf103 | G | 0.929 | 0.948 | 0.0445796 | A | 0.073 | 0.030 | 0.009065 |
| (SEQ ID NO: 69) | | | (SEQ ID NO: 70) | | | | | | | | |
| rs17105987 | 12 | 67652474 | CPM | T | 0.974 | 0.989 | 0.00274107 | C | 0.025 | 0.014 | 0.2535 |
| (SEQ ID NO: 71) | | | (SEQ ID NO: 72) | | | | | | | | |
| rs12310897 | 12 | 53213084 | NCKAP1L | T | 0.885 | 0.920 | 0.00280375 | G | 0.121 | 0.062 | 0.007115 |
| (SEQ ID NO: 73) | | | (SEQ ID NO: 74) | | | | | | | | |
| rs7166489 | 15 | 99037892 | ASB7 | T | 0.786 | 0.826 | 0.0091569 | C | 0.172 | 0.149 | 0.4062 |
| (SEQ ID NO: 75) | | | (SEQ ID NO: 76) | | | | | | | | |
| rs2561540 | 19 | 45881373 | NUMBL | T | 0.070 | 0.047 | 0.0157695 | T | 0.080 | 0.035 | 0.01138 |
| (SEQ ID NO: 77) | | | (SEQ ID NO: 78) | | | | | | | | |
| rs3857079 | 4 | 149313918 | NR3C2 | T | 0.629 | 0.681 | 0.00670933 | n.a. | n.a. | n.a. | n.a. |
| (SEQ ID NO: 79) | | | (SEQ ID NO: 80) | | | | | | | | |
| rs1630816 | 10 | 118518583 | HSPA12A | G | 0.192 | 0.236 | 0.00322308 | G | 0.220 | 0.270 | 0.1268 |
| (SEQ ID NO: 81) | | | (SEQ ID NO: 82) | | | | | | | | |
| rs17051171 | 4 | 132412783 | LOC646187 | G | 0.039 | 0.022 | 0.0139119 | G | 0.038 | 0.030 | 0.5395 |
| (SEQ ID NO: 83) | | | (SEQ ID NO: 84) | | | | | | | | |
| rs17011412 | 4 | 127826471 | LOC132817 | G | 0.890 | 0.853 | 0.00663146 | A | 0.127 | 0.143 | 0.5466 |
| (SEQ ID NO: 85) | | | (SEQ ID NO: 86) | | | | | | | | |
| rs8071556 | 17 | 69016542 | LOC728073 | G | 0.680 | 0.736 | 0.00249514 | C | 0.334 | 0.257 | 0.02612 |
| (SEQ ID NO: 87) | | | (SEQ ID NO: 88) | | | | | | | | |
| rs10761618 | 10 | 51244612 | NCOA4 | T | 0.666 | 0.724 | 0.00172485 | C | 0.277 | 0.287 | 0.7851 |
| (SEQ ID NO: 89) | | | (SEQ ID NO: 90) | | | | | | | | |
| rs1431079 | 2 | 226123709 | KIAA1486 | T | 0.677 | 0.714 | 0.0367737 | A | 0.334 | 0.289 | 0.2027 |
| (SEQ ID NO: 91) | | | (SEQ ID NO: 92) | | | | | | | | |
| rs38619 | 7 | 76279172 | FDPSL2B | G | 0.007 | 0.015 | 0.0373197 | G | 0.035 | 0.046 | 0.4728 |
| (SEQ ID NO: 93) | | | (SEQ ID NO: 94) | | | | | | | | |
| rs6129628 | 20 | 34803775 | NDRG3 | T | 0.936 | 0.953 | 0.0458609 | G | 0.042 | 0.016 | 0.04422 |
| (SEQ ID NO: 95) | | | (SEQ ID NO: 96) | | | | | | | | |
| rs2240164 | 19 | 965712 | C19orf6 | G | 0.797 | 0.760 | 0.00601433 | n.a. | n.a. | n.a. | n.a. |
| (SEQ ID NO: 97) | | | (SEQ ID NO: 98) | | | | | | | | |
| rs12653596 | 5 | 24955849 | LOC729826 | C | 0.961 | 0.981 | 0.000820889 | A | 0.067 | 0.035 | 0.05697 |
| (SEQ ID NO: 99) | | | (SEQ ID NO: 100) | | | | | | | | |

| | | | | SWE Replication Sample | | | | | |
|---|---|---|---|---|---|---|---|---|---|
| | | | | (451 RBP+ and 297 RBP− SLE cases) | | | | | |
| | | | | | | Allele | | Replication P | Meta P value |
| | | | | | Allele frequency | frequency | | value | (GWAS, US and |
| SNP | Chr. | Position | Locuse | Allele | RBP+ | RBP− | P value | (US and SWE) | SWE) |
| rs1005715 | 16 | 73071533 | GLG1 | C | 0.210 | 0.165 | 0.03225 | 0.003781 | 0.0005507 |
| (SEQ ID NO: 63) | | | (SEQ ID NO: 64) | | | | | | |
| rs4838288 | 9 | 127510162 | MAPKAP1 | T | 0.090 | 0.069 | 0.1508 | 0.007564 | 5.85E−05 |
| (SEQ ID NO: 65) | | | (SEQ ID NO: 66) | | | | | | |
| rs1419617 | 7 | 125076596 | LOC646841 | T | 0.138 | 0.103 | 0.04076 | 0.01148 | 0.001323 |
| (SEQ ID NO: 67) | | | (SEQ ID NO: 68) | | | | | | |
| rs7775840 | 6 | 146942309 | C6orf103 | A | 0.048 | 0.037 | 0.3235 | 0.02263 | 0.002505 |
| (SEQ ID NO: 69) | | | (SEQ ID NO: 70) | | | | | | |
| rs17105987 | 12 | 67652474 | CPM | C | 0.027 | 0.012 | 0.04893 | 0.02315 | 0.0001836 |
| (SEQ ID NO: 71) | | | (SEQ ID NO: 72) | | | | | | |
| rs12310897 | 12 | 53213084 | NCKAP1L | G | 0.112 | 0.098 | 0.3789 | 0.02532 | 0.0002055 |
| (SEQ ID NO: 73) | | | (SEQ ID NO: 74) | | | | | | |
| rs7166489 | 15 | 99037892 | ASB7 | C | 0.185 | 0.143 | 0.0335 | 0.02598 | 0.000615 |
| (SEQ ID NO: 75) | | | (SEQ ID NO: 76) | | | | | | |
| rs2561540 | 19 | 45881373 | NUMBL | T | 0.068 | 0.056 | 0.3465 | 0.02799 | 0.001097 |
| (SEQ ID NO: 77) | | | (SEQ ID NO: 78) | | | | | | |
| rs3857079 | 4 | 149313918 | NR3C2 | G | 0.393 | 0.337 | 0.02887 | 0.02887 | 0.0004994 |
| (SEQ ID NO: 79) | | | (SEQ ID NO: 80) | | | | | | |
| rs1630816 | 10 | 118518583 | HSPA12A | G | 0.230 | 0.264 | 0.1246 | 0.03338 | 0.0003103 |
| (SEQ ID NO: 81) | | | (SEQ ID NO: 82) | | | | | | |
| rs17051171 | 4 | 132412783 | LOC646187 | G | 0.051 | 0.029 | 0.03498 | 0.03658 | 0.00126 |
| (SEQ ID NO: 83) | | | (SEQ ID NO: 84) | | | | | | |
| rs17011412 | 4 | 127826471 | LOC132817 | A | 0.109 | 0.145 | 0.03733 | 0.03916 | 0.0006909 |
| (SEQ ID NO: 85) | | | (SEQ ID NO: 86) | | | | | | |
| rs8071556 | 17 | 69016542 | LOC728073 | C | 0.305 | 0.281 | 0.3249 | 0.03926 | 0.0002955 |
| (SEQ ID NO: 87) | | | (SEQ ID NO: 88) | | | | | | |
| rs10761618 | 10 | 51244612 | NCOA4 | C | 0.298 | 0.235 | 0.007682 | 0.03983 | 0.0002192 |
| (SEQ ID NO: 89) | | | (SEQ ID NO: 90) | | | | | | |
| rs1431079 | 2 | 226123709 | KIAA1486 | A | 0.299 | 0.261 | 0.1072 | 0.04056 | 0.003458 |
| (SEQ ID NO: 91) | | | (SEQ ID NO: 92) | | | | | | |
| rs38619 | 7 | 76279172 | FDPSL2B | G | 0.019 | 0.035 | 0.0471 | 0.04067 | 0.003517 |
| (SEQ ID NO: 93) | | | (SEQ ID NO: 94) | | | | | | |
| rs6129628 | 20 | 34803775 | NDRG3 | n.a. | n.a. | n.a. | n.a. | 0.04422 | 0.006952 |
| (SEQ ID NO: 95) | | | (SEQ ID NO: 96) | | | | | | |
| rs2240164 | 19 | 965712 | C19orf6 | A | 0.174 | 0.216 | 0.04425 | 0.04425 | 0.0006641 |
| (SEQ ID NO: 97) | | | (SEQ ID NO: 98) | | | | | | |
| rs12653596 | 5 | 24955849 | LOC729826 | A | 0.057 | 0.044 | 0.2741 | 0.04858 | 0.000148 |
| (SEQ ID NO: 99) | | | (SEQ ID NO: 100) | | | | | | |

1-11. (canceled)

12. A method of diagnosing or prognosing lupus in a subject, the method comprising detecting in a biological sample derived from the subject the presence of a variation in each of at least three SLE risk loci as set forth in Table 2, wherein:
(a) the biological sample is known to comprise, or suspected of comprising, nucleic acid comprising at least three SLE risk loci as set forth in Table 2, each locus comprising a variation;
(b) the variation at each locus comprises, or is located at a nucleotide position corresponding to, a SNP as set forth in Table 2; and
(c) the presence of the variation at each locus is a diagnosis or prognosis of lupus in the subject.

13. A method of aiding in the diagnosis or prognosis of lupus in a subject, the method comprising detecting in a biological sample derived from the subject the presence of a variation in each of at least three SLE risk loci as set forth in Table 2, wherein:
(a) the biological sample is known to comprise, or suspected of comprising, nucleic acid comprising at least three SLE risk loci as set forth in Table 2, each locus comprising a variation;
(b) the variation at each locus comprises, or is located at a nucleotide position corresponding to, a SNP as set forth in Table 2; and
(c) the presence of the variation at each locus is a diagnosis or prognosis of lupus in the subject.

14. The method of claim 12 or 13, wherein a variation is detected in at least four loci, or at least five loci, or at least seven loci, or at least ten loci, or at least 12 loci, or in 16 loci.

15-18. (canceled)

19. The method of any one of claims 1, 7, 12, or 13, wherein the three SLE risk loci are PTTG1, ATG5, and UBE2L3.

20-35. (canceled)

36. A method of diagnosing or prognosing a subphenotype of lupus in a subject, the method comprising detecting in a biological sample derived from the subject the presence of a variation in each of at least three SLE risk loci, wherein:
(a) the biological sample is known to comprise, or suspected of comprising, nucleic acid comprising at least three SLE risk loci selected from HLA-DR3, HLA-DR2, TNFSF4, IRAK1, STAT4, UBE2L3, and IRF5, each locus comprising a variation;
(b) the variation at each locus comprises, or is located at a nucleotide position corresponding to, a SNP as set forth in Table 2; and
(c) the presence of the variation at each locus is a diagnosis or prognosis of the subphenotype of lupus in the subject.

37. A method of aiding in the diagnosis or prognosis of a subphenotype of lupus in a subject, the method comprising detecting in a biological sample derived from the subject the presence of a variation in each of at least three SLE risk loci, wherein:
(a) the biological sample is known to comprise, or suspected of comprising, nucleic acid comprising at least three SLE risk loci selected from HLA-DR3, HLA-DR2, TNFSF4, IRAK1, STAT4, UBE2L3, and IRF5, each locus comprising a variation;
(b) the variation at each locus comprises, or is located at a nucleotide position corresponding to, a SNP as set forth in Table 2; and
(c) the presence of the variation at each locus is a diagnosis or prognosis of the subphenotype of lupus in the subject.

38-40. (canceled)

41. The method of claim 36 or 37, wherein the subphenotype of lupus is characterized at least in part by higher levels of interferon inducible gene expression in a biological sample derived from the subject as compared to one or more control subjects.

42-57. (canceled)

58. A method for selecting a patient suffering from lupus for treatment with a lupus therapeutic agent comprising detecting the presence of a genetic variation at a nucleotide position corresponding to a single nucleotide polymorphism (SNP) as set forth in Table 2 in each of at least three SLE risk loci selected from HLA-DR3, HLA-DR2, TNFSF4, IRAK1, STAT4, UBE2L3, and IRF5.

59. The method of claim 58, wherein a variation is detected in at least four loci or at least five loci, or in 7 loci.

60-61. (canceled)

62. The method of claim 59, wherein each variation comprises a SNP as set forth Table 2.

63. The method of claim 62, wherein the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

64. The method of claim 58, wherein the lupus is a subphenotype of lupus characterized at least in part by the presence of autoantibodies in a biological sample derived from the patient to one or more RNA binding proteins for treatment and/or by a higher level of interferon inducible gene expression as compared to one or more control subjects.

65. (canceled)

66. A method of assessing whether a subject is at risk of developing lupus, the method comprising detecting in a biological sample obtained from the subject, the presence of a genetic signature indicative of risk of developing lupus, wherein said genetic signature comprises a set of at least three single nucleotide polymorphisms (SNPs), each SNP occurring in a SLE risk locus as set forth in Table 2.

67. The method of claim 66, wherein the genetic signature comprises a set of at least four SNPs, or at least five SNPs, or at least, seven SNPs, or at least ten SNPs, or at least 12 SNPs, or 16 SNPs.

68. (canceled)

69. The method of claim 66, wherein the SLE risk loci are selected from HLA-DR3, HLA-DR2, TNFSF4, IRAK1, STAT4, UBE2L3, and IRF5.

70. The method of claim 66, wherein the SLE risk loci are PTTG1, ATG5, and UBE2L3.

71. A method of diagnosing lupus in a subject, the method comprising detecting in a biological sample obtained from said subject, the presence of a genetic signature indicative of lupus, wherein said genetic signature comprises a set of at least three single nucleotide polymorphisms (SNPs), each SNP occurring in a SLE risk locus as set forth in Table 2.

72. The method of claim 71, wherein the genetic signature comprises a set of at least four SNPs, or at least five SNPs, or at least seven SNPs, or at least ten SNPs, or at least 12 SNPs, or 16 SNPs.

73. (canceled)

74. The method of claim 71, wherein the SLE risk loci are selected from HLA-DR3, HLA-DR2, TNFSF4, IRAK1, STAT4, UBE2L3, and IRF5.

75. The method of claim 71, wherein the SLE risk loci are PTTG1, ATG5, and UBE2L3.

76-77. (canceled)

78. A method of assessing whether a subject is at risk of developing lupus characterized by the higher levels of interferon inducible gene expression compared to control subjects, the method comprising detecting in a biological sample obtained from the subject, the presence of a genetic signature indicative of the risk, wherein said genetic signature comprises a set of at least three single nucleotide polymorphisms (SNPs), each SNP occurring in a SLE risk locus, wherein each SLE risk locus is selected from HLA-DR3, HLA-DR2, TNFSF4, IRAK1, STAT4, UBE2L3, and IRF5.

79-128. (canceled)

129. A method of prognosing lupus in a subject, the method comprising detecting in a biological sample derived from the subject the presence of a variation in each of HLA-DR3, HLA-DR2, TNFSF4, IRAK1, STAT4, UBE2L3, and IRF5, wherein the variation at each locus comprises, or is located at a nucleotide position corresponding to, a SNP as set forth in Table 2, wherein the presence of the variation in at least two loci indicates a prognosis of lupus in the subject.

130. The method of claim 129, wherein the presence of the variation in at least three loci, or at least four loci, or at least five loci, or at least six loci indicates a prognosis of lupus in the subject.

131. The method of claim 129, where the prognosis is an increased risk of earlier age of diagnosis of lupus in the subject compared to a subject lacking the presence of the variation in at least two loci.

132. A method of aiding prognosis of lupus in a subject, the method comprising detecting in a biological sample derived from the subject the presence of a variation in each of HLA-DR3, HLA-DR2, TNFSF4, IRAK1, STAT4, UBE2L3, and IRF5, wherein the variation at each locus comprises, or is located at a nucleotide position corresponding to, a SNP as set forth in Table 2, wherein the presence of the variation in at least two loci indicates a prognosis of lupus in the subject.

133. The method of claim 132, wherein the presence of the variation in at least three loci, or at least four loci, or at least five loci, or at least six loci indicates a prognosis of lupus in the subject.

134. The method of claim 132, where the prognosis is an increased risk of earlier age of diagnosis of lupus in the subject compared to a subject lacking the presence of the variation in at least two loci.

135. A method of prognosing a subphenotype of lupus in a subject, the method comprising detecting in a biological sample derived from the subject the presence of a variation in each of HLA-DR3, HLA-DR2, TNFSF4, IRAK1, STAT4, UBE2L3, and IRF5, wherein the variation at each locus comprises, or is located at a nucleotide position corresponding to, a SNP as set forth in Table 2, wherein the presence of the variation in at least two loci indicates a prognosis of the subphenotype of lupus in the subject.

136. The method of claim 135, wherein the presence of the variation in at least three loci, or at least four loci, or at least five, loci, or at least six loci indicates a prognosis of the subphenotype of lupus in the subject.

137. The method of claim 135, where the prognosis is an increased risk of earlier age of diagnosis of the subphenotype of lupus compared to a subject lacking the presence of the variation in at least two loci.

138. The method of claim 135, wherein the subphenotype of lupus is characterized at least in part by higher levels of interferon inducible gene expression in a biological sample derived from the subject as compared to one or more control subjects.

139. A method of aiding prognosis of a subphenotype of lupus in a subject, the method comprising detecting in a biological sample derived from the subject the presence of a variation in each of HLA-DR3, HLA-DR2, TNFSF4, IRAK1, STAT4, UBE2L3, and IRF5, wherein the variation at each locus comprises, or is located at a nucleotide position corresponding to, a SNP as set forth in Table 2, wherein the presence of the variation in at least two loci indicates a prognosis of the subphenotype of lupus in the subject.

140. The method of claim 139, wherein the presence of the variation in at least three loci, or at least four loci, or at least five loci, or at least six loci indicates a prognosis of the subphenotype of lupus in the subject.

141. The method of claim 139, where the prognosis is an increased risk of earlier age of diagnosis of the subphenotype of lupus compared to a subject lacking the presence of the variation in at least two loci.

142. The method of claim 139, wherein the subphenotype of lupus is characterized at least in part by higher levels of interferon inducible gene expression in a biological sample derived from the subject as compared to one or more control subjects.


SEQUENCE LISTING (100 sequences)

SEQ ID NO: 1 - DNA, 52 bp, Homo sapiens
aaccacaataaatgattcaggtgtccrtacaggaagtggaggggggatttca

SEQ ID NO: 2 - DNA, 801 bp, Homo sapiens
tctagttaattcacacttcattttattttttgagacagggtcttgctctgtcacccagactggagtgcagtggcacaatcatggcttactgcagcccccaactcctgggctcaagtgatcctctcacctccaccatccaaatagttgggactacacatgcatgctgctattgctctgctaaattaaaaaaaaaaaattagagatgaggtctcactatgttgcgcaggctagtcttgaactcctggcctcaatgaactcctcaaactcaaggctcacacatcagcttcccaaagtgctggaattaaaggcatgagccaccatgcccatcccacactttattttatacttactgaactgtactcaccagcttcctcaaccacaataaatgattcaggtgtccrtacaggaagtggaggggggatttcatcatctatccttggagcagttgctatccaaaatgtcaaaaatattgtaacaattgttaattagaacaatccaaaggaaattcttatattctaatattaaatataaatttaccataatttatatttaaattccgttgaagcaacattatcagtaaagttgacacttgttcattcaaggaaaaagcaaaataaattctcttaaggtacaaacccaggaggtttttgcttattctttaacatttattgttttaaaagctcaaaagtagggctgggcgcggtggctcacgcctgtaatcccagcactttcggaggccgaggcgggcagatcacaaggtcaggagatcgaaaccatcctggctaacgtggtgaaaccgtg

SEQ ID NO: 3 - DNA, 52 bp, Homo sapiens
gtatgaaaagttggtgaccaaaatgtkaatagtggttatcttatttcagtgg

SEQ ID NO: 4 - DNA, 632 bp, Homo sapiens
caacacacatgtacacattatatttttaaaaatatccacaaattatatcttttccatttttttccatttttatttaattaggtaaacatagatacacatatggaaaggttcacacttgactgttaatacggatgtctttgaaggtagtggtgtggatggaggtaaggaaaaaagaagtgggataaaaagaagtttgtaattaaaaagctacatgtatattatgatctactttatggaaaattacatgagtgtgtatgcagtaaaagtatgaaaagttggtgaccaaaatgtkaatagtggttatcttatttcagtggaatttcaggggattttttttctttcttcttagacttttcattatcatttgactttttacaaagatttgcattatttaagcaatcagaaagaaattataaagctattttcatcataacaaaaattccattggtaaaaaatttttaattaatttacataatgtgcaaaaattagaaaattagaactcctaaagcaagaagtggaaaaattattccaatctgaagaaataaaaccattctctgatgactgctgacatttacgaagcaactctcaaagtgtccctgtacattttccatggaacaaaaatctgaagtcta

SEQ ID NO: 5 - DNA, 52 bp, Homo sapiens
agctgcgcctggaaagcgagctcgggkgggtgcctacagcagggtgcgcccg

SEQ ID NO: 6 - DNA, 1000 bp, Homo sapiens
gtggccagtctagggcaccgcgccgtctggcatctccctggaggccctgggcctggcccgaggctcagcccggatctgcagttgccaggtcagtgcggggcccggagtggattcgcggggcggggcggggcactgcccgcgcccggagctcagcagcagctgcccaggggcgggggcggcaagacgcggaagtgcccggcaggttggcggaccggcgggaggcgcagcctgggcagagctcagcttggtcccgccgcccggccggtgctccctggcgcagccacgcaggcgcaccgcagacaggtgggtcccggccgccgcgctctcctctctgcgtccgcgcccggcgcgccccgagggtggcgagagcggtgcccgctactgcccccaagtctaggcctagactgggccccgcgccccccaggcactgcgggcggcgggatgaagactggagtagggcggggtccgcgtccagctgcgcctggaaagcgagctcgggkgggtgcctacagcagggtgcgcccggccggcctgggacttccaaagcgcctcccacgccccgatcggtttggggtgctggcgcccggggagcccagtgacccaggcggcggagtgggcagcgctgcggggggcgccggctctgctgctctccctccccctcgccatcgcccagaatgggggttcccgggagccccgctggaggctggcttggaccacagaggagcgaggcccgatccttactttcgatgcactcgcccttgctcttaccgggccaccctcaccctttcggaaaagaggttgaggttaaagcgttcatcccccgggatcttcaggccaatggcaggaactgtgcaagagtttgggggaagatggtgtcaggtagaggctgcgtccctgggctcgcggccgggaatggcagactctcgtcccccgagcagcggaaaaggatggggcgcaatagttcctgggctggtttcctcaggtcctgtcccagaactta

SEQ ID NO: 7 - DNA, 52 bp, Homo sapiens
taagattaaacacttatcagatcattrtctgcttttggtttttctagtaccc

SEQ ID NO: 8 - DNA, 701 bp, Homo sapiens
gctactagttttctttttatctatttatttatttaatttatgtttgaggtggagtctcgctctgtcacccaggctggattgcagtggtgcgatctcagctcactgcaacctctgcctcccgggttcaagcgattcctgtgcctcagcctccctagtagctaggactacaggtacccaccaccacaccgaaataatttttgtatttttagtagagatggggtttcaccatgttgtccaggctggtctcgaactcctgacctcaagtgatctgccccgccctcagcctcccaaagtgttgtgattgcaggcttaagccaccgctccctgcctagctactggttttcagtgacagccctcaaaccaagtccaccattcccattaggtaacctgtgcttttcctcactcaggccaggggctggggagcttcaggcaagatgtccgtagactcatggccattctgatgcaggccatttttaagattaaacacttatcagatcattrtctgcttttggtttttctagtacccagaaacaaacattttctagtacccagaaattgttgtttctgacaattttgtctagttttatacttgttttcttcaaaaagaaattggccaacttcttagacccttctgaggtgaatcttggactggattggttttatgtttcttccacttactttattgcctacagcttttgatt

SEQ ID NO: 9 - DNA, 52 bp, Homo sapiens
cagcacaggctcatgcgagcccatccrcctgcagggtgagtcactgccccgc

SEQ ID NO: 10 - DNA, 601 bp, Homo sapiens
ttttgtgtcattcttaggaagaccttttctcattctgagtcttaacattttcccatttttttctttgctaattcttcctcctagtcataggaatttaggaatccgggtatgggcccccaccgtcctctgggtggcagaacttcctctgtggtctccttctctccccacatgtcgaagttttctctgttcccacttctccccacagggtggtggttggagccccccaggagatagtggctgccaaccaaaggggcagcctctaccagtgcgactacagcacaggctcatgcgagcccatccrcctgcagggtgagtcactgccccgccgggctgggactgggattcccctgtgaacacatagggactttccaggcactcctgtgtcctggggatctgtggtggggacacaggtgcctgcctccgtaccctctcctctgcctgcagtctctaccctagacatccccaggcaacccctctgtgttcctttctttcccaagatttaggatcccccttcaccgtcagacctccttgtctcccgcatggaggtgacccctgcccagctcttccacagccttctctgtccccccaccagggtgaccat

SEQ ID NO: 11 - DNA, 52 bp, Homo sapiens
cctggcagcctccacggttcccttccrcttccagctccatcactgactgcat

SEQ ID NO: 12 - DNA, 601 bp, Homo sapiens
accttgatctgcagtgctctgtgaccgcagcctgagcagacgtacaccgtgcggacagaactgtgcacagccaccgccgagtgattcatcttcaaagcttgctgtgtgttctgttacagaaggagtgagtacaggttcttccttctctgcagtggctcacttggagaatttccgtcttgtctgaagtcaactcaaaagtcaatgcgttctcctaggtaacacccaagcacacaggaaccggccgtggaagcccccagtgaaattcctggacaggcctggcagcctccacggttcccttccrcttccagctccatcactgactgcattccgaagatcagactagaaattgttacacgcttcacgtttggctgtgacgagctctgagcagtgaccttcacagctccctttccctggtgcagaccaggaggcagcgctggcagcatgcccactgcgctgaccttagtgcagtgccactggctgggaagtctctgggccagagttcctgaggctggggggctggggaagggcaactccccagagaaaggcttggagaaagggcctttgggctctgatcaccagccctggggtctcaggttccaga

SEQ ID NO: 13 - DNA, 52 bp, Homo sapiens
ccgatcgcccgcacgctgccggacctytcctgtgaccttaacctctccaagc

SEQ ID NO: 14 - DNA, 501 bp, Homo sapiens
cggactctgcttcctcctgggccccacccagctcacccagaggctcccggtgtccaggcagggtgagtgtcgggaccaccaaggctttgaggagctcacgcacatccaattgggggtgcggtgggctagagacagtcttgccagagtggatcagaaagaagggatctggaaaaagagttaccacgtgttgcactggttcctgacgctgctgcccgcacatcctgccgatcgcccgcacgctgccggacctytcctgtgaccttaacctctccaagcctcagtttcttcatctgttggatggggataataacacacccagcactgaaagcaacacaggatgattcatggccaggggttagcacagcagctagcaccaggcgacagccccatgaaggccagctgttgttatttttagaggagaggatctattttcatccaatgggtcctgggatatgaccaattggtttgtgccgtagtttaggaaaggtcagtgaaagtgca

SEQ ID NO: 15 - DNA, 52 bp, Homo sapiens
tgctaaccatgtaccttaaataaaccrtcttctagttttttgtttcttactc

SEQ ID NO: 16 - DNA, 801 bp, Homo sapiens
agcttaagtcttggaatggtttattttttagatgccatttagccacttatattctcttctattttattgtgagaactaattcccctcttacattctgtgcttgacccatgctatacttagtgtgaacaagagccaccttcttctcatgacttctatttttttgtgaaaatttccttcactcattcacgacatttggatttgaaatcttacctacttaagtactttaaaaaatcattttctaccatctttcttatcaggaggctctagtgattccttctccacacttctaacttctcatcttcacactccttgtcttcctaacttcactacagtaagtgttttacatgtttagaactcagctcctttactatgattgctaaccatgtaccttaaataaaccrtcttctagttttttgtttcttactctcaattataccttttagaaaagaattaagagtagaaaaagactgctacatagacattcttatgatcttcagaaatgagcacagatcatgcttaatgaaaaaagatttccaaacaatgctgcatatgtccagagaaaaggtggcagaaatgactgtcgtttgggggcactattgtctggacatggccagttctcagaactccagtccctaaattcccttctaactaaaggaaaagcctcttaagggtcttatagaaatcctgccactttcacctgaaagaataatcttcagttatgtggcacatggccaagagtaaaagtctttagtcacttggaagcagacagacactgtaatgctaaataattggacataacat

SEQ ID NO: 17 - DNA, 52 bp, Homo sapiens
gacacatatgaggcagctgagagtaartgaggaccatgtggtaaaatgattg

SEQ ID NO: 18 - DNA, 801 bp, Homo sapiens
tagagatgaggtcttgctatgttgcccaggctggtctcaagctcccaggctcaaatgatcctcctgcctcggcctcccaaagtgctgggattacaggcatgagccaccttgtctggcccacttttctttttataggactttattagatgaggtgaagaacaggtaatttgggttgataatttgagaataaaatgatttattttaccaattttttctacttctcaattttaaaataaaatgatcccttattttgctaaagaaaagaaatgtgaagctatatctgtaatagaaatatgtaaaaaaaattgacattcatgttttcactatttacaaagcttagccacatgcccattttatttgattacttgtgaggtgacacatatgaggcagctgagagtaartgaggaccatgtggtaaaatgattgttgaaggcattcagcctgctggactcctttacccactccccacctgtgccagttcccatgtggaaatgtgagtaagtctgagagaaagaaacgggcaacattttaaatatctatagagtacataattgatggaatgaccccaagatctaccaccggagacatccgaaaagcttcaaaagttgttcagggaaatttgagaatgacacttcataaatttttaataataaaaaattatccactgatggaaacctcactaatttttgaggcaatcatgatggaacgacatagagacctccaggccgtagtcactgaatataatataaacagaatagcgtccttagtgaacacaacagcatagataacaaaaagagtagg

SEQ ID NO: 19 - DNA, 52 bp, Homo sapiens
aaggctgcttccatagctagtctagcygaaccatttccgagctacaaggcag

SEQ ID NO: 20 - DNA, 601 bp, Homo sapiens
ttttaaagaccaataaatctgcaggtcaggaaactgtctacttgggtggccatagtagaaaaaccataccacactgtttaactgaaagagtaacccaactccctttcccactgaggctacacaaaagggtaaaatgtaagcgtgatgagctttggtgtgaattaggagaataaaaagtgcagaatgaaccacttccctgaagtctgaagggtctgtggcatcagggcaggggcctgccctttcaactccatccccagatgtctatcaggtaccaaaggctgcttccatagctagtctagcygaaccatttccgagctacaaggcagtgaatgaaagtaaaaacaaagaaacactggttaaattttaaaaatttattctttctcttttgttgctgttgatttgttcttgagatggctacaacaccagacagaacagtgccctcatatctgatggctgtgaagggctgcactcctttgaaacattaagatctgctttgggcttccctcttcggcctcaccctccccttaaaatcaagtcatttccacactttttagtaacatactaaatgacacatcatcccttgttagccctgtaaacattt

SEQ ID NO: 21 - DNA, 52 bp, Homo sapiens
atgcagaactcactatgttgtaactaygatctgtaatgataaaaatatagaa

SEQ ID NO: 22 - DNA, 601 bp, Homo sapiens
agttgatcacagcataatgaatcctcaatttccagagtactagagatgattatatacaaaaatccagtgaaacttattttactaatttaccagttctatatcactcctaaatttccctaaaaatatggatttataaaaggtagtattctataattcacaaatatgaagagctttattataatttgagtatgaactgtgtatcacccatatcatggcttcagaaaactattgcttttcttactaacttatttaaggtttcactacatgagtcagtatgcagaactcactatgttgtaactaygatctgtaatgataaaaatatagaacctctttgactttaatctaaaaaagtcaccttatcataaaacagtgttaacataatcaaaaattaaccctgacaaaaaaaaacccacttattttggcttttaaaaatagctcatgctcatggttctttcaaagtggtgttgtagctaccgtcttttctaacagaggaatattttcatcatagcaactatttctgagccaaaaataaaccacactaaaaacagaagaaaattctcagtgtagaaaggaaaaggctcatctgttgtgcaatagcagg

SEQ ID NO: 23 - DNA, 52 bp, Homo sapiens
cattggtggggctgaaataaaaaaccycgatttagaaatctgatacaaaagc

SEQ ID NO: 24 - DNA, 601 bp, Homo sapiens
ttacataacatttacaagcatcagaaccaacatcaaaattaaactggctcttactgtgtcctaagggtgagacaagggttttaagaactgaaacttgggagctattatgattggcaaatggaaggatgggaaaagtctacagataaggttctgtacttaagaatgacccactttgtcatacacgtatatctactttcatatggatatttgcacaagggtttctttttttttctttttttaagagggggtgaaagaaggaactcagtgcacatgacattggtggggctgaaataaaaaaccycgatttagaaatctgatacaaaagcaaagtcatcgttttcaaatcaaaacttggctctgcttcattggcttgcctcgtgcttctgcacaggctttcgggggaggcttccaggcacatgtgcctagaggccatccttctaagtgtgcacactggagctgtaagggcaagggtggctgctacctcacattggcactgagaaatatccggagagtgactccttggcatgaaattcaaacaccttatgtggtgatctggtcccacttcagcccactcctcatctcccatcctccatccagccaa

SEQ ID NO: 25 - DNA, 52 bp, Homo sapiens
tcacttcagtcagctaggaaagatacmcttttggcagggtgcggtggctcac

SEQ ID NO: 26 - DNA, 701 bp, Homo sapiens
aaacaatgtaggctgccatcttgatctgtgcttttccccagggacatgagcaagggcacttctaggccaagcaaaatcaatcgtgtttcatagatttggatttagagggcgctctaaaccagctcagaggaatggtatgttttagaatcctagggaaattgagttctccaatcatcacttcagtcagctaggaaagatacmcttttggcagggtgcggtggctcacgcctgtaatcccagcactttgggaggtcgatcacaaggtccggagttcaagaccagcctggccaatatggtgaaaccctgtctctactaaaaaatacaaaaattagccaggtgtagtggcatgtgcttgtagtcccagctacttgggaggctgaggcaggagagtcgctggaaccagggaggcggagttgcagtgagccgagattgtgccacactccagcctgggcaacagagcgagactctgtctcaaaaaaataaataaataaaaaagatatccttttaataaagaaaaaaaaacaacacataaaacttaagagtgtaataatagctgctgttcattgaatgcttaccaggaactagtatgatcataaggaactcttacaatcaccctgagagggagggcatggcaagccctaattaatggatgaagaaactaaggctcaaagaggtcaagccacttttccaggcaacatggc

SEQ ID NO: 27 - DNA, 52 bp, Homo sapiens
taaaaataacagctgggttaaaaagakttcttcatgacaccgtgatactgag

SEQ ID NO: 28 - DNA, 649 bp, Homo sapiens
atcttaatttatgattaaaggaaatagtgagacttgtggatacaatcaggtaacagtcactggctatgcatcttttcagtgctgtgtaagttaatggaaagcatatgagtataagactgtcttttccacattgagaacactagatgtcaagaggaaagttgctcctacgagttttaaaaataacagctgggttaaaaagakttcttcatgacaccgtgatactgagaatgatgataataaccattcaaacaatttcatatagttagaacttccagtaagtagttcctggatcccccaaatttgagtaaacacttacctgacacatggaagacattaaatgacagttatcattttattattttttggatatgtattattttttgagtgctccctaaacgtttgacactgtaataaacacgaggaatataaaaataaatgtgaccatcaaggagtctagaaaagggagaaaggcacatacactaatcatgtgaatgccagacagacattgctatgctggaggaattcacagagaacatgaagaaaggcatctagctcaatttggatgggtggctagaaaggcttcctgtaggaacaatatttgagaagaatcttaagggaatagtgacaatagagtacgtaatttgttggg

SEQ ID NO: 29 - DNA, 52 bp, Homo sapiens
gagcaggcaacttgtgaattgcaagtygattgcattggtggctttcctggca

SEQ ID NO: 30 - DNA, 801 bp, Homo sapiens
cagggtttcatcatgttggccagactgggtcttgaactcctgacctcaggtgatccgcccgcctcggcctcccaaagtgctgggattacagacgtgagccaccacgcctggcccagtttaacattttttgataaaagtaaatttcatttcattccaagaaaggtgttagacaactgctcgcctcactggccctctggagcctgccctgccctgcttcccccagaggaatacaatccctggggacttatgacaactgaacagtgcagaaggaaccagcagagggaaccgctgcctttcttcatcactgccggcagcaaaagctgactggaagccatgtcctagtccagatccgggcatgcacaccggctggtgttgagcaggcaacttgtgaattgcaagtygattgcattggtggctttcctggcagagctttcggatctcagttcagacaaactcgtcagcacaccctgtcctgacctccccctaaactagggctggccctcttccgaccccttcttggcaccgtgcactttccttccctttgtagtgcttattatcataatgcaaagtctatgtctcccccactagacccgtgagccatgtgagggcggggtggccactgacgccatttgaaatcaggggagtcctcattgaatcattgcttttactctgggtaaaaagccagggctggaggggctggagtcggggttctctctggtacagcagtatactcccagggcctgggacagcacagatgtataggaaacatctggcaaaaggagagctggaatccatgtctgc

SEQ ID NO: 31 - DNA, 52 bp, Homo sapiens
gtgggatggagaaggtgggatccaaaygggagaatttctgggattttccatt

SEQ ID NO: 32 - DNA, 601 bp, Homo sapiens
tccaagctctggcccctacttgttggtcaatacttagccaggcttccaccccactcctctttgctccagtgcccaattttgctgctatgggctttctcagacctccatgtaggcccatgtgacctcagcccttgtccatcccctcttctcccctccctacatcttggcagactccccataccttggacagtgatggtcacaggcttggatgagaacagcgtgtagcctatgtttcctgtgcagtggtaatcaccactgtgactgtggtttgcttgtgggatggagaaggtgggatccaaaygggagaatttctgggattttccattctggaagaatgtgaccttgaccagaggcttgtccttccagctgtggcacctcagcatgatggtttctccctcctggaactccaggtgaggggtctggagcaccagccattctgaaagacacaaatatgataagaaaaagttgtaaggatagattccaagggtttttcagtctcagaggtacgttactcacagaacttgacatgatgtctggcagacagaaatgaagatgcttcatgacagatgtgagcattctcttataggcaatatatggtatt

SEQ ID NO: 33 - DNA, 52 bp, Homo sapiens
cgcctcctgcccccccgagtgtgtccrggcctcttctaagcccctgcaaaac

SEQ ID NO: 34 - DNA, 601 bp, Homo sapiens
gcacgctccgtgcccgtttgcagactctctctcaggctgagccccgcacgctctgtgcccgtttgcagacaccctctcaggctgagcctgacatgctccgtgcccgtttgcagacactctctcagactgtttaggaaatccttctcttttcttatcacagagctacccatgctttcttttcaaaggttcagctttgctttctagtccgagatcttgactccgtccggcatcgtgtgtgtgctgtgagcagcccccagctgacctgccccacaggcgcctcctgcccccccgagtgtgtccrggcctcttctaagcccctgcaaaacccctgggactggtttccattcctggaccggttccagttcaatccctgtagttttataaatcataatatccagtaggcaatttctccccttttcaaagctatcttggcaattcttggtgccttcctgtcctcctgtgttgacttcacaacacatcaagcaccctgaactcctgctgggaaggacggaatcagcatcctcaacctctctatggagcaagatgtctcgaaagggaaggggccttgcttcagtcacggctgtgttagaaaagctcttta

SEQ ID NO: 35 - DNA, 52 bp, Homo sapiens
atatttcattctgtgacttggggtgtkgttttgctatccaggatccttagag

SEQ ID NO: 36 - DNA, 1783 bp, Homo sapiens
ttactggccccaattctttcttctttactcatatagaaggttgaatcctttgcaggtgaactttttaaattttaattttttttttcttctgaggtggagtttcactcgttgcccaggccggagttcaatggagcgatcttggctcactgcaacctcctgcctcctgggttcaagcgattctcctgcctcagcctcctgagtagctgggattacaggcgtgtgccaccatgctgggctaattttgtatttttagtagagacggggtttctccatgttggtcaggctggtctcgaactcccgacctcaggtgatccgcccgccttggcctcccaaagtgctgggattacaggcatgagccaccgtgtctggcctaaatttttttttttttttttgagacagagtctcgctctgtcacccaggctggagtgcagtggggtgatctcagctcactgcaacctccacctcccgggttcaagcgattcttctgccttagcctcccgagtagctgggactacaggtgtcaccaccacgcctggctaatttttgtatttttagtagagatggggtttcaccatattggccaggctggtgttgaactcctgaccttgtgttctgcctgccttggcctcccaaagtgctggaattacaggcgtgagccaccacacccggcctcggcctaaattttaaattttaagttttattttatttttatttttttgagacaaggtctcactctgtcacccagactggagtgcagtggcatgatcatgggctcaggcaatccttccgcttcattttttaatttttttgtagagatgaggtcttactgtgtggcccaggctggtctccaactcctggcctcaagtgattttcccgcttcggcaacccaaagtgctgggattacaggagcactttgggaggccgaggcgggtggatcccttgaggttaggagttggagaccagcctggccaacatggcaaaaccccatctctactaaaaatacaaaaatgagctgggcatggtggtgcgtgcctgtagccccagctactcaggaggctgaggcataagaagcacttgagcctgggaggcggaggttggagtgagctgagatcacgcctccacactccagcctggagagcgagactccgtctcaaaacaaacaaacaaaaaacaacaaaatgtggaattactggcataagccacttcgtccaaccagcaggtgaactttttaacgtttgaagacagatgtatgtcatcttcacatttctttaggccataggttcctggaccttttgattgttcctggtaggacatggtccttgctgttcttgtcctgagcagcccttatttgatgtggtcttctaaagtatggtgtccagaactggacacagcatttggctctgaatagttcagggtggcctggacgctaaacatggcacaaacgattggtcagctaaaccagtcagtttttcatgctgctgctaaggttcatgccttgtgcttggtgaaaaattaagtgtttggaaccaagtccaggtctttactggatctctaaaatatttcattctgtgacttggggtgtkgttttgctatccaggatccttagaggtttggtctttaagcctgtattcgttccttccctgatatgtggtacctaactacatctctctcagcccacttcccaccagggtgacttcaggccaccagttctaaagccattcgattctcctcagctcttcttgatccagcctctgtttatccattttgtacagaaacattgtag

SEQ ID NO: 37 - DNA, 52 bp, Homo sapiens
cttactgtaagttaccaaatggagcaktgagttgttacaaggattccattcc

SEQ ID NO: 38 - DNA, 701 bp, Homo sapiens
tttcagagaggggaaacagatgtctacttggcaggcaccaagtttttctccacagtagaatcacctggggagcgttagaaaaatttctcatgcccaggctgcgtgcatgctgattaaatcggagtcttgtggttggagcttgtagtctgtaaagcttcccaagtgattccaatatggagccaagtttgaaaaccacagcccttgaggctggtgactcacagtgtggttctctgaccagcagcattggcatcactgggagcttgttgaaggtgcagaattcagtcctcatcccagacttcctgaatcaggatctgattttaacaaggtccccaggtgatttgcacgcacattaaagcgtgagaagcctcacgctaggatgtacctgccatttcctaaccgaagtgctctacagggctaaggaaaatgcctttctagagtgtaaccaagagaaggaaagtttatttcaaactcacacttactgtaagttaccaaatggagcaktgagttgttacaaggattccattcctgccttcacttgttttttttgttcacggaagagaaaagatagcacagataggactggggctctagggacagcttgaattctgtcaaagtagcatcttttgtggctttcatttctcagtgtttcaccttgattgttcccaaaattgtcagacagcaggccatctggactgacttgt

SEQ ID NO: 39 - DNA, 52 bp, Homo sapiens
aatgccgaaaagagaaattctccaagygatataacaggatggcttccctttt

SEQ ID NO: 40 - DNA, 601 bp, Homo sapiens
ttggcaacactacctttgaatatgatactctggattacagagatgtagtcttcaggttcctgttcagttgagatctcccatctgctttgagagatatttaataattcatagagctgatctgaactcttcactccacaaagcaaagtaactacactttttggtgaatgaagtatcttttccagaaactgacatttctttggagttaggtctctaagcaggctatttgataatatcaaaagtttacatttgtaagacgttaagttcagcaactccaaatgccgaaaagagaaattctccaagygatataacaggatggcttcccttttcacaacatgtaaaaatacttctgtcaagtacagagcccattcctcagcatcttcttcatatatcattattatatcttttgtatttcctggaaaaagaaaaaatattttcttatatttgcaatgctatcggcaggttaaattattatttctcaaaaattttaatatgctcactaggcaatatattaatttgcttataccagagaaaaactctatgaaagtccttcatagtataaaaaatcatagattaaataaaactagaattcaaagacaagttg

SEQ ID NO: 41 - DNA, 52 bp, Homo sapiens
atgcttcttcacccctctgaacccctrgctctgcaagttcctctgcttccgc

SEQ ID NO: 42 - DNA, 801 bp, Homo sapiens
ctctgacaaacgttactattgaacgagagtcacactgcctggctgcccctgaggacctgtcaccaaagccactcactcgtctgcctgccgctgacccccccaggcctctccaaagttcagcaaccaaagagtcaggcctggtacagaagggttgtcaggctgaggcccattctgctggttggtgcctgggcctgagtcaccatctagaacattcccaagtttgaactcaggtacggtgctcagtctctccaggaatcagtttcaacaagataatcctttgctcacccttctcatggaattaaatgaggctgtctgcaaatgtcatttgaaaactgctaacctttttgggatcatttaaaaggcagaactgaaacatgcttcttcacccctctgaacccctrgctctgcaagttcctctgcttccgcagctattccatccccagataaagacttgaggtgtggagaggatagagggcctgctaccttgagaacttcccactcagggcagcgacctctgtacacggtcatttcaagcacacctggtctcagtgttcattgtttatgttccccccgaccccaccctgggcacatacattttcctgctccatgagggatccttgtccctgccctccctgccctgtagagataggagttgctcttacttacttgatcaaatacacatcatacttcccagcagagcggccagatttcctttgcttaagcttccgtgtccagccttcaggcagggtggggtcatcatacatgggtccccggtcacggatgatggagcgccgctgtttg

SEQ ID NO: 43 - DNA, 52 bp, Homo sapiens
gcctattgccccaggatcaaatagacytctcacaagatggtagttcccctta

SEQ ID NO: 44 - DNA, 1001 bp, Homo sapiens
atcacgtgagtccctaaaagcagaggacctttccagaagtagatgtgacaatgggcaaatagtcagagagagatggagtgatgtggtttttgaagatggagggaggaaccatgagccactgaacatgagcaggctgtagagactggcaaaggcagggaagcagagtgtcccctgcagtctaccaaaggagtgcagccctgctgtgagactcatgtacttccaatctacaaaactgtaaaataataaatgtttgttgtttaaagccactaattttgtggtactttgttatattagaaagctaagacacatccctaattttgaaacagtttctataagctttatgaaattaaccaggtaagaagttggtgggtggggggggggggggagcacaaaaataaaccaagcttgtggcacattccacattaatcatgaggtcaactttctctctgacctccttgctcatagttgcttaatgcctattgccccaggatcaaatagacytctcacaagatggtagttccccttaactactctatagacaacaacttaagcactgtgaattgttaagttccatttgagatattcttccacgtcctacacatcaataaaactactgatccagctgatctgaaggacccaacagaagccaactcaccaaagaatgcagtttccacagcctgattatttcatcccccttatcccaaccaatcaataatcccattttccagccccttaccctccacagtcctcttaagagcttcagcccagaactccttgggcaagtggatttgcgggtctcttcccatctcctcactcaccaccctgtgattattaaattctttctctactgcaaaccctgctgtctcagtgtaattggtctgttactatgcagtggatacatgaacctgttggtccaattttggcaagaataaaaataggcatctagctatccccaaaacaaaacatgaaaaagctagtcacaacttccacctggcaaaaac

SEQ ID NO: 45 - DNA, 52 bp, Homo sapiens
cctctaacctcaaggcaatgagagacrtctttgagtaaagatgtgaactggg

SEQ ID NO: 46 - DNA, 3938 bp, Homo sapiens
ccatattttgttgttttttttagagacagagtctcactctgttacccagattggagtacagtggctcaatcatagctcactgcaacctgaaacacctgggctcaagtgatcatcctgcctgagcctcccaagttgtgtcagttaatttttttttaatttttaattttttttattttttatttttattttttgtagagacagggtctcactaagttgcttaggccagtctcaaactattgacctcaagcaactcccaccccacccctggactcccaaaacactgggactacagatgtgagccaccacacccaatcctattccatatttggtaatggtcttggtcatagtatatatacaatatcctgcccagcactctactcagttatactagtgactgctagggcacccaatataccacagacattaatcttcaatgctttctctcacatttgtgttactctgtgctgggaatattaacacctctaacctcaaggcaatgagagacrtctttgagtaaagatgtgaactgggaaattattacaggcccagagtaatatggtttggatctgtgtccccacccaaatctcatgttgaattgtcatccccaatgttggagaaggggcctagtggaaggtgactggatcatgggggcagactctccccttgccgttcttgtgattgcgagtgagttctcacaagatttggttgtttaaaagtgtgtatcagctgggctcagtggctcatgcctgtaatcccagcagtttgggaggccaaggcaagcagatcacttgaggtcaggagtttgagacgagcatggccaacatggtgaaaccctgtctctactaaaaatacaaaaattagctgggcatggtgatgcatgcctgtaatcccagctactcggaaggctgaggcaggagaatcacttgaatctggaggcagaggttgcagtgagctgagatcgtgccactgctctccatcctgggtgacagagtgagactttgtctcaaaaaaataaaacataaaagtgtgtagcgcctccttgttcactatcttccttctgctccggccacataagaggtgccttgcttccccttcaccttccaccatgattgcaagtttctctttggtcatgcttcctatacagcctgtggaaccatgagccaataaaacctcttttctttataaactacccagtctcaggtagctctttataacaatgtgaaaatggactaatacagaaaatttgtgccaagaagtgggacattgctataaagataactgaaaatgtggaagcaactttggaactgggcaacaggcaaagtctgaaagagtctggagggctcagaagaaggcaggaagatgagggagagattggaacttcctagagacttgttaaattgttgtgaccaaaatactgatagtgatacagtcagtgaagtccaggctgaggagatctcagatggagatgaggaacttattgggaactggagcaaaggtaacttctgttacacattagcaaagagattggaggcattgtgcccctgccctagggatttgtggaactttgaacttgagagtgatgatttagggtatctggtggaagaaatttttaagcagtaaaacattcaagatgtagcctggctgcatctaacagcgtatggtgataggcataagcaaagaggttatctgaaactggaatttatgtttaaaagggaagcaaagcataaaagtttagaaaatttgtaacctgaccatgtggtagaaaagaaaaacccattttggggggaggaattcgagccagctgcagaaatttgcacaagtaatgaggagtctagtgttattagccaagataatgggaaaatttcttgaaggcattccagagaccttggtggaagcctctcgcattacaaacctggtggcctaggagggaagaatggtttcgtgggctgggcccaggcctctgctgccctgcacagcctcaggacactgctccctgtgtcccagccactctagctccagccgtggctaaaatggccccagatatgtctcaggctgctgctccagagagtacaagccataagccttggcagctccacgtggtgttaagcccacaggtgcatagaggacaagagttgaggcttgggagcctctgcctagatttcagaggatatacagacatgcaaggatgtgcaggcagaagtctgctgcaggtgtggagccctcatggagaacctctactagggcaatgtggaggggaaaatgtgggattggagcccccacacagagtcgctacttgggcattgcctaatggtgctgtgagaagacagccaccatcctccaggctccgtgcacttggaaaagctgcaggcactcaacaccagcctatgaaagcagctgaggaggctgtaccctgcagagctaaaggggcagagatgcccaaggccttggaagcctgccccttactgatgggtgggctggatatgagacatggagtcaaaggaaattaatttagagatttaatatttaatgactgccctactgggtttcagacttgcatggggcctatagaatcttagttttggatgatttctcctttttggaatggatgtatttacccaatgcctataccctcattgtatcttggaagtaactaactataactaacttgttttttatttacaggctcataggtggaagggacttgccttgtctcagatgagactttggaatgtggacttttgagttaatgctgaaatgagttaagactggggggattgtagaaaagtgatgattatattttgcaatgtgagaaggacttgagatttgggaggggacaggggtggaaaagatatggtttggatttgtgtccctgcccaaatctcatgttgagttgtaacccccagtgttggaggaagggcctggtgggaggtgattggatcatgggggcagatctcccccttgctgttctcatgatagtgagttctcttgagatctggttgcttaaaattatgtagcacctcctgctttgctctccttctcctgctccagccatgtaagatgagcctgctttcctctcaccttccaccattattgtaagtttcctgaggcttcctcagtcaggcttgctgtgcagcatatgaaactgtgagccaataaaactgcttttctttataaactactcagtcgcaggtagttctttataacaatgtgagaatggactaatgcacagagtaatctgtatcagagcataatgcaacagagtgtggactctggaatcagatgcctggtttggatctgtttctgttctttaccagctatgtgatctagaacaagattgttattctctctgctttgattttctcatctgttaaatgagtatataataattcgtaactcatagtaattctgtgaggaataatgagttaaaataagtaaagtgcttagaatagtgcctcacagagtaactgttattttaggtattagttatcattttactcatttattttttattttatttattttctttttagaaggagtcttacttttttggccaggctggagtgcactagcatgatctcggctcactggaacctcttcttcctgggctcaaacgattctcctgcctaagcttcccaagtaactgggattacaggtgcacaccaccacacccagctaattttttgtatcttcagtagagataggatttcactttgttggccaggctggtcttgaactcctgacctcaagttatccatctgcctcagcctcccaaagtgctgggattacaggcatgagccactgtgcctggccttattcatttattttttattgaggtaaaatagtacccattattattgtatgtgctattggctctggttacagggactgactggaaagggagtgacttccatgaccagcccaaatagaggttggttttt

SEQ ID NO: 47 - DNA, 52 bp, Homo sapiens
tcctctcttcctactattactattatycctaaaacaatttggctgttgcagc

SEQ ID NO: 48 - DNA, 501 bp, Homo sapiens
ctcccctcataaccacacttgagcagctctcagacaatccttttgtaatgtaaaccactcactcctccttttcaaccccttcccctgttggcccattagcagggactcaattgttggctccctgtcaccacaatcacagttaagtcactattgtgcttctcccaacggaatggctgcagttccaactatttaaaattattattaataattcctgtagtaattgctcctctcttcctactattactattatycctaaaacaatttggctgttgcagctcaagacaggtgctaaatatgtacttgttgattgataaatggatgtacataaggttacactaaaaaaactagagctccttagctaggaaattcaaaggctcaggggagtaatttgaaaaggtttataaaaccacaagcaaaattgatactgagcacatggacttgtttattaaatattagaattttgagccaaggggaacacacacacacacacacacacacaca

SEQ ID NO: 49 - DNA, 52 bp, Homo sapiens
attaaaaataataaaagcaacaaaaaragaaaaagtacagctttgaagccag

SEQ ID NO: 50 - DNA, 836 bp, Homo sapiens
aagatcacttttgtttcagatagacaccacccacctctatttccccaggcttcctttctcttcctcatgtctttctaagctctctaattcagaaaactcatggatctggagcttcctccacagaaaggcagcaagaccttgcatcagtaaagaagtgcagcttttaggctgggcgtggtggctcattcctataatcccagcactttgggaggctcacgtgggaggatcacctgaggtcaggagttcgagaccagcctggccaatatggtgaaaccctgtctctactaaaaacacaaaagttagccaggtgtggtggtgggcacctgtaatcccagctacctgggaggctgaggcagaagaattgcttgggcccgggaggcagagtttgcagtgagcagagatcgcgccattgcactccagcctgggtgacagagtgagactccatctcaaaaaacaaacaaacaaaaaagaagtgcagcttttagatagaaggaatagattctaggttctacagcactgtagggtgactataattaaagacaatgtattgtatattccaaatagccggaagagcaaattttgaatgttcccaacacaaagaaataatacattaaaaataataaaagcaacaaaaaragaaaaagtacagctttgaagccagaccaagttccggttgtacctgagacctttgattacctagcacaggttcatctctgggttttactttcctcatacacaaagtaccagggacaaagtaggcacatacgtgtttatcccttttctaccccccacgtcttttgtctctgaccctcatagacatttatagctactctgtt

SEQ ID NO: 51 - DNA, 52 bp, Homo sapiens
aatgtaagctgtctagtccaccataargcacttatgtacctcccttagaaac

SEQ ID NO: 52 - DNA, 601 bp, Homo sapiens
accttccccgcggattctcttgggtgaaactcacagtggcctgggaagaaaggctcatggggagactgagctagcttcacagatggagttaagtggggagagggaggtaaatgcaaaagctctttgcaaacctaactgcatttggtaaattgaaaaagggctttctaaaccgtctgtggggcacataataaagttctttgtgtggagcaaagttcttgctcaagtataagatactctctgcaaagtgttccagttgtaatggaatgttccgtaaaatgtaagctgtctagtccaccataargcacttatgtacctcccttagaaaccgtaagttgcacactgtgaagtcctttccaaagggcacaatgcaattctgagagcacaccgagtgcagtgcagcgctcagcactgtagtgcaggccgggactgctgacgctttgggatctgtcatgcactttgtaagtgatgactgcacagacatcgtgaagggttttggaaaggacaaggaactttagaaaataccacactctctggccacaccaggcacctgggccttcgacaggagggcctcatcagagacaggaggagcccccgcccaccc

SEQ ID NO: 53 - DNA, 52 bp, Homo sapiens
gtcctttatttgaacgcctaagatagrgtgcccattgccaccgccatccgat

SEQ ID NO: 54 - DNA, 501 bp, Homo sapiens
tctttcctttcaaaaaaaaaaaaaaagcagggatgaaaattaccttcgctagttgacagagcacccaagcgctaacgatgccaagtctgggtgccggccccctgggagtgacccgctgcggcagcgccgccaggctctggctcccgaagtcctgccctcaaattcgcgtcccgggctcgcttactttctgttctgttccccggtgcttgggtgtttgcttcctagtcctttatttgaacgcctaagatagrgtgcccattgccaccgccatccgatcacttttgctcccttcggaccccccaaccccacttcctgagttgtgggggggcgtttgtccctgggagagagggaggaggggactcggtgactgaagcaggaagaagagaggctaggggaggggtggagacagctaaaagcaaagagacagctgctgaccctgggaggagaagagccccaggcagtggtttttttgttgttgttgttgttttgtttgtttgtttg

SEQ ID NO: 55 - DNA, 52 bp, Homo sapiens
gggggaggggggagggatagcattagragatatacctaatgctaaatgatga

SEQ ID NO: 56 - DNA, 511 bp, Homo sapiens
tgctatgtctgtgatcaggcacacattttactggacttttactgtcagggccgtcatttagtgccaagatgtctagagagttcttaataagtgtactcaattggctgagaaaatgtgtccatgcaaaaaaccaaacaccgcatgttctcactcatagatgggaattgaacaatgagaatacttggacacaggaaggggaacatcacactctggggactgttgtggggtggggggaggggggagggatagcattagragatatacctaatgctaaatgatgagttaatgggtgcagcacaccagcatggcacatgtatacatatgtaactaacctgcacattgtgcacatgtaccctaaaacttaaagtataataataataaaaaaatgtgtccatggctctgggaggagcatgtttgttttcctcatttcccagtctgtaaataagcaaattgaaaggggttagtgataatgtccatctccagaagctgtcagatttcctttgtcaaactc

SEQ ID NO: 57 - DNA, 52 bp, Homo sapiens
taataccacttgttactttggctgcarcgctggattcacactcataggagac

SEQ ID NO: 58 - DNA, 511 bp, Homo sapiens
taaatagctcttgcaggagcctcccttgttatgaagggagtgaaaagcagttattccagattagccttaactctgttaacgcttcttttaattgggaggtctctgaatggaaacattttctcacaacaggcatagcatcacttcctactccaggggtgcaatgtccagccctcaccactcctgggcagggaaccatgtactgtaggcatcatccgggaacctttggttttaataccacttgttactttggctgcarcgctggattcacactcataggagacagcactctcagctgcagaccttcaggacaatggacagcagtaactccagcatgcagaggtaaggtgaaaaggagcaggcagttctgaacctgccttcctgcaagtactcaagctagccattgtgcctgtatgttgaagtctcacccaaccacaaccactactccaagtgtcatctcccttatgaaatccacaccaatcccattagaagtaatgtgtcacaggcatttcac

SEQ ID NO: 59 - DNA, 52 bp, Homo sapiens
atgatctggggccccagcccacctgcrgtctccgggggtgcccggcccatgt

SEQ ID NO: 60 - DNA, 1003 bp, Homo sapiens
ccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcrgtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcgtagtggggggcccaggttcggtgccggtactgcagccaggctgtggagccgcgggcctccttcctgcggtgggccgtggggctgactccctctccctt

SEQ ID NO: 61 - DNA, 52 bp, Homo sapiens
ccgcggcctgtctgccggctggccgamtgccttgtgagccttggccttcttc

SEQ ID NO: 62 - DNA, 601 bp, Homo sapiens
gcacaggtagtggctggagtcggccgtcaggcggaaatagccgtccaccagcgacacgaaggacagcgccgcagcccgggaaggcaagctcagctcctgccagccaggggcgcatcaggtgggtgtcctcccaggccatgatgggccctagcccagcccctaccctgggcctcaccaggcacttgttgtcctgccggtggatgctgacacagtgctctttcagcaccacgtgggtgatgtcccggaagtcacagaagtaggcccacagtggctcccgcggcctgtctgccggctggccgamtgccttgtgagccttggccttcttcccaaacaggctggcttgggggttcctgccactgctgccactagaaccctgaacacccagcaagtggggcagaggatggagagggcaatgcccgtttatacctcctgcactttcccctggggagagactggagccagaagggccaggcaccaacctaggttgacctcccaagtcccagtgtcacaggtgggatggggacccaccccaaaatcatacacatgctggaagccggacccttagggaaggcagagagaagaatcctgaccccctcacacc

SEQ ID NO: 63 - DNA, 52 bp, Homo sapiens
agtgcagcaatcttctccccacaaaayagtctaaaaagttttcctaatatga

SEQ ID NO: 64 - DNA, 709 bp, Homo sapiens
ttaaacctaaataaagttaggacagcagaatttaactgtccatttgtgattcagattttgggaattaaaaaaaaaaatctggaaatcttcccccacccactttgttaaaaacataaaatcagatgatagaagctggtagggcaactctaagtgcactcatggaaggaagaagtttctagaagtgcagcaatcttctccccacaaaayagtctaaaaagttttcctaatatgactgaaatcagaagaatatacagcaaacaatttaaaaaacaaatgacaagaagaagaaatgaagtcgatattcactgcataccctccttccctgttcctcagtgcggtaggcgtgtctgtataaacaagagaacacagctccctgaggcataaattcactggtctcattccaaccgtgggtgtggcaaagacgagaagcgtctccctggcacttgcggtacaggacagggtccagcctataaggttaagagttaaaagataaccactaattaatagacatttccttctaacacaggctcagggttttttatgaggtgtttgcttgtatactcattgataatcacttccaggggcgtggagacagaagatttgtaaaagtatattcctgaattttgtccttttggttttttttttttgacacgggatctctcaatctgtcacccaggctggagtgcagtggtgctatcttgactcactg

SEQ ID NO: 65 - DNA, 52 bp, Homo sapiens
gagggacccctgcccaattttaaactyattaataaatatgccatggggccag

SEQ ID NO: 66 - DNA, 1025 bp, Homo sapiens
atgctgctcccttcctctctcacggaggaaatcagtagagaaagccacgatgaagggggaaaaaggattcagggagtgcagtgagagtggttctcccacctgaaagatgcttggggaagtctctcaaaaacgcaccttagcaaaagaaaaaactcaaggttgaatttcagccctgagggacccctgcccaattttaaactyattaataaatatgccatggggccaggcacggtggctcacacctgtaatcccagcactttgggaggccgaggagggcagatcacttgagatcaggagttcgagaccagcctggccaacatggtgaaaccctgtctctactaaaaatacaaaaattagtctggcgtggtggtgtgcacctgtagtcccagctactcaggagactgaggcaggagaatggcttgaacccgggaggcagaggttgcagtgacccaagatcaggccactgcactccagcctgggtgacagagactctgtctcaaaacaaaaaaacaaaaaacacggtgggtctacccatgtatttcaaatatggtcatccatcacttaatgatgaggacacaagaaatgcatcgttagctgattttgtggttgtgtaaacatcatagactgtacttacacaatgtcagagcctactacacacctaggctgtttggtattagcctattgcttctaggctgcacacctgtacagcatgttactgtaccatatactgtaggcaattctaacacaatggtatttgtgtatctaaacatctctaaatgcagaaaaggtacaattaaaagaccatgttataatcttaggggaccaccatcatatgcactctgtcattgaccaaaacatcctcattccacgcatgaccatatgtgattgatctcttactacatcccattactttatgtaattggtatcttaccacatatctatactgggggaatagtgcacctcagcacccagacacactcttgcacacacaccaccacagttccatcagggacctgtcagaggca

SEQ ID NO: 67 - DNA, 52 bp, Homo sapiens
acactgtgatttttcttgaatcttcaycgtccgaacttaatagggattggga

SEQ ID NO: 68 - DNA, 565 bp, Homo sapiens
actatgtaaaagccttttttcccaggataatagacctttgttatgaaggcgtacttcacaatgattaatcttccccttccacttccaaagccacactgtgatttttcttgaatcttcaycgtccgaacttaatagggattgggaaggtaaaatgatgaaagtgaggaatgccctgaagaacgtggcccctgggagttactcacactaatacacactcagcctttaacaattcatcaaagttgtcatttacatatttttactagttcatagctgcagaggatactgcttcaggtaaacacatcttgactgtggctctctggatttacctgtctccccagataccagagaggaagttgtcctgcaacctacattctctggtgtatctaagaaaatttacaaatgtttggtttgttgaccattttctaggagtagaaagaaatgacttcaacataataaaatccatatgtgaaaaacacacagggaatattagactcaatgtgaaagtctaaaaaatttacctccaacatcaggatcaaggcaagcatacccacttttgccatttcta

SEQ ID NO: 69 - DNA, 52 bp, Homo sapiens
gacaatcggtctagtttcgagtctatrgaaacagaatttaaaggataatctg

SEQ ID NO: 70 - DNA, 1707 bp, Homo sapiens
cagactctaaaatcctataaaacaacatctgtgatagataatcttcaagccagacataataatctaaataataaaaataatgtttcagagaaaaattaagtattcgtaacactttttaaaattaaacttctcaaacaaagtcttggctagaatgccatgttacatttctaacaagacaatcggtctagtttcgagtctatrgaaacagaatttaaaggataatctgaagattgctatgagattccaccattttcagtaattcaagaagataaatttttaaagttttcgagctatgcattgtagcaaaaaaatccttttatttatttatttattgttatacattaagttctgggatacatgtgcagaaagtacaggtttgttacataagtgtacacctgccatggtggtttgctgcacctgtcaacccatcatctacattaggtatttctcccaatgctatccctccccaaccccccacccccaaacaggcccagtgtgtggtgttcccctccctgtgtccatgtgttctcattgttcagctcccacttatgagtgagaacatgtggtgttttgttctctgttcctgtgttagtttgctgagaatgatggtttccagcttcatctatgtccctacaaaggacatgaactcattctttttatggctgcatagtattccatggtgtatatgtgcaacattttctttatccagtctgtcattgatgggcatctgggttggttccaagtctttgatattgtgaatagtgctgcaataaacatacgtgtgcatgtgtcctttgggtatatatccagtaatgggatggctgggtcaaatggtatttctggttctagatccttgaggaattgccacactgtcttccacagtggttgaactaatttacactcccaccaacagtgtaaaagtgttcctatttcttcacatcctctccagcatctgttgtttcctgactttttaatgatcgccattctaactggcatgagatggtatctcattgtggttttgatttgcatttctctaatgaccagtgatgatgagttttttttcatatttttttggccacataaatgtcttcttttgagaagtgtctgtttgtatccttcacccactttttgatggggttgtttgtttttttcttataaatttgttggagttttttgtagattcaggatattagccatttgtcagatggatagattgcaaaaattttctcccattctgtaggttgtctgttcactctgaatgcctacaggagaaagtgggaaagatctaaaattgacactctaacatcacaatgaaaagaattagagaagcaagagcaaataaattctaaagctagcagaagaaaaaaaatagctaagatcagagcagaactgaaggagatagatacatgaaaaacccttcaaaaaatcaatgaatccaggagctggttttttggaaagattgacaaaatagatagactgctagctagagaatcataaagaagaaaagagagaagaatcaaatagacataataaaaaatgacaaaggagatatcaccactgatcccaccaaaatacaaactaccatcagagaatactataaaaattccttttcgaagacactactgaaaaaaataatttttccttgacttatattctgctttgcaaaaccttaaaaaatatatttgtgatattaagtgtactactctg

SEQ ID NO: 71 - DNA, 52 bp, Homo sapiens
caatcttaaaacctttcattattttaygataaggtagcactttagacgggag

SEQ ID NO: 72 - DNA, 700 bp, Homo sapiens
ctgtgtaaataaaatttcttaatggcccaacatattagttacaattacttttatggcatttaggaaaaagaggaaattaaaaagtccttatatttgaagtatcagcataaatgaaaatgtctttggcaagaaatgattataataaatttgtagggcattacatttctgttttttttttgagatggagtctcgctctgtcacccaggctgaagtgcagtggcacaatctccgctcactgcaagctccgcctcctgggtgcacgccattctcctgcctcagcctcccgagtagctgagactataggcgcccgccaccacgcctggctaattttttttgtatttttagtagagactgggtttcaccatgttagccaggatggtctcaatctcctgacctcgtgatctgcccatctcagcctcccaaagtgctgggattacaggcgtgagccaccgcacccagcctacatttcttagcaatcttaaaacctttcattattttaygataaggtagcactttagacgggagaaggttttagaccctgagttttgatatgagattatattatactacattgtctaggaatgaaaaaaaatcatatcatagtagtcatcatctattgtatcacagtagtatgatagtagtttgaggtctgtctcatcaaagcatggatgaaaatgttcttcatttgaccagaatgtcc

SEQ ID NO: 73 - DNA, 52 bp, Homo sapiens
aagttatgtgttaagaaaatcttcatkactttgagatcaaaagagttgtgaa

SEQ ID NO: 74 - DNA, 401 bp, Homo sapiens
gtcatcttgtcttttgaatcatctggaactaagaagtatgctatggcaggaaatacagctagagcctaggaatagaatccttcaaatgcatagggcaggctccttctcttataattgattcctttaatggagagtggtctgtgttaagaggaaactagttagcaggaagaattaaagttatgtgttaagaaaatcttcatkactttgagatcaaaagagttgtgaaatttccacctctatctgcctaaaatcatgaggggcaagtgactgtgggggcagtgagggggcagtgtttcactaggatagctgcccctcttgaaagatctttgattttaagctgagcctgggtttctggcacctgctgatgttggtccaaatgctctaggctgtcaggagcttgt

SEQ ID NO: 75 - DNA, 52 bp, Homo sapiens
ggtgggactgcctcactcctctggtgygtgggttgctttgagctgatactat

SEQ ID NO: 76 - DNA, 7197 bp, Homo sapiens
atgaggttttgaatccattttttggtatgtgaaagttttttttatatatactttaagttctagggtacatgtgcacaacgtgcaggtttgttacataggtatacatgtgccatgttggtttgctgcacccattaacttgtcatttacattaggtatatctcctaatgctatcccttcccccaccacaggccccagtgtgtgatgttccccgccccatgtccacgtgttctcattgttcaattcccacctatgagtgagaacatgcagtgtttggttttctgtccttgtgatagtttgctgagaatgatagtttccagcttcatccatgtctctgcaaaggatatgaactcatcttttttatggctgcatagtattccttggtgtatatatgccacattttcttaaaccagtctatcattaatgaacatttgagttggttccaagactttgctattgtgaatagtgccacaataaacatatgtgtgcatgtgtctttatagtagcatgatttataatcctttgggtgtatacccagtaatgggatcactgggtcaaatggtatttccagttctagatccctgaggaatcgccacactgacttccacaatggttgaactagtttacagtcccaccaacagtgtaaaagtgttcctgtttctccacatcctctccagcacctgttgtttcctgactttttaatgattgccattctaactggtgtgagatggtatctcactgtggttttgatttgcatttctctgatggccagtgatgatgagcattttttcatgtgtctgttggctgcataaatgtcttcttttgagaagtgtctgttcatatcctttgcccactttttgatgaggttgtttgtttttttcttgtaaatttgtttaagttctttgtagattctggatattacccctttatcagatgggtagattacaaaaattttctcccattctgtaggttgcctgttcactctgatggtagtttcttttgctgtgcagaagctctttagtttaattagatcccatttgtcaattttggcttttgttgccattgcttttggtgttttagtcatgaagtccttgcccatgcctatgtcctgaatgctattgcctaggttttcttctagggtttttatggttttaggtctaacatttaagtctttaatccatcttgaattaatttttgtataaggtgtaaggaaggggtccagttttggctttctacatatggctagccagttttcccagcaccatttattagatagggaatcctttccccatttcttgtttttgtcaggtttgtcaaagatcagatgattgtagatgtgtggtgttatttctgaggcctctgttctgttccattggtctatatctctgttttggtaccagtaccatgctgttttggttactgtagccttgtcatatagtttgaagtcaagtagtgtgatgcctcgagctttgttctttttgcttaggattgtcttggcaatttgggctcttttttggttccatataaagtttaaagtagttttttctaactctgtgaagaaagtcattggtagcttgatggggatgacattgaatctataaattaccttgggcagtatggccattgtcacgatattgagtcttcctatccataagcatagaatgttcttccatttgtctgtgcccacttttatttcattgagcagtggtttgtagttctccttgaagagggccttcacatcccttgtaagctggattcctaggtattttattctctttgtagcaattgtgaaagggagttcactcatgatttggctctctgtttgtctgttattggtgtataggaatacttgtgatttttgtacattgattttgtatcctgagactttgctgaagttgcttatcagcttaaggagattttaggctgagatgatggggttttctagatatacaatcatatcatctgcaatcagggacaatttgacttcctcttttcctaattgaataccctttatttatttctcctgcctgattgccctggccagaactgccaatactatgttgaataggagtggtgagagagggcatccttgtcttgtgccggttttcaaagggaatgcttccagtttttgcccattcagtatgatattggctgtgggtttgtcataaatagctcttattattttgagatatgttacatcaatatctagtttattgagagtttttagcatgaagggctgttgacctttgttgaaggccttttctgcatccgttgagataatcgtgtggtttttgtcgttggttctgtttatgtgatggattacatttattgatttgcgtatgttgaaccagccttgcatcccagggatgaagctgactttattgtggtggataagctttttgatgtgctgctggattcagtttgccagtattttattgaggattttcacatcaatgttcatcagggatattggtctaaaattctctttttttgttgtgctctgccaggctttggtatcaagatgatgttggcctcataaaatgagttaggaaggattctctctttttctattgattggaataatttcagaaggaatggtaccagcttctctttgtacttctggtagaattcggctgtgaatctgtctgatcctggattttttttggttggtaggctattaattattgcctcaatttcagagcctgttattggtctattgagagattcaacttcttcctggtttagtcttgggagggtgtacatgtccaggaatttatccatttcttctagattttctagtttatttgcgtagaggtgtttatagtattctctgatggtaatttgtatttctgtgggatcggtggtgataagccctttatcattttttattgcatctttttgattcttctctcttttcttctttattagtcttgctagcggtctatcaattttgttgatcttttcaaaaaaccagctcctggattcactgattttttgaagggatttttgtgtctctgtctccttcagttctgctctgatcttagttatttcttgccttctgctagcttttgaatgtgtttgctcttgcttctctagttcttttaattgtgatgttagggtgtcaattttagatctttcctgctttctcttgtgggcatttagtgctataaatttccctctacacactgctttaaatgtgtcccagagattctgatatgttgtgtctttgttctcattggtttcaaagaacatctttatttctgccttcaattcgatatttacccagtagtcattcaggagcaggttgttcatttcccatgtatttgtgcagttttgagtgagtttcttaatcctgagttctaattgattgcactgtggtctgagagacagtttgttgtgatttctgttcttttacatttgctgaggagtgttttacttccaactatgtggtcaattttggactaagtatgatgtggtactgagaagaatgtatattctgttgatttggggtggagagttctgtagatgtctattaggtccgcttggtgcagagctgagttcaagtcctgggtatccttgttaacctctgtctcgttgatctgtctaatacagagagtggggtgttaaagtctcccattattaatgtgtgggagtctaagtctctttgtaggtctctaaggacttgctttatgaatctgggtgctcctatattgggtgcatatatacttaggatagttagctcttcttgtcgaattgatccctttaccattatgtaatggccttctttgtctcttttgatctttgttggtttaaagtctgttttatcagaggttaggattgcaacccctgcttttttttgttttccatttgcttggtagatctttctccatccctttattttgagcctatgtgtgtctctgcatgtgagatgggtctcctgaatacagcacactgatgggtcttgactctttatccaatttgtgtctgtgtcttttaattgggacatttagcccattttcatttaaggttaatattgttatgtgtggatttgatcctgtcattatgatgttagctggttattttgcccattagttgatgcagtttcttcctagcctcgatggtctttacaatttggcatgtttttgcagtggctggtaccggttgttcctttccatgttcagtgcttccttcaggaactcttgtaagtcaggcccggtggtgacaaaatctctcagcatttgcttgtctttaaaggattttatttctccttcacttatgaagcttagtttggctggatatgaaattctgggttgaaaattctttttttaagaatgttgaatattggtccccactctcttctggcttatagagtttctgccaagatatctgttgttagtctgatgggcttccctttgtgggtaacctgacctttctctctggctgcccttaacattttttccttcatttcaactttggtgaatctgacaattatgtgtcttggggttgctcttttcaaggagtatctttgtggtgttctctgtatttcctgaatttgaatgctggcctgccttgctatgttggggaagttctcctggataatatcgtgaaaagtgttttccaacttggttctgttctccctgtcactttcaggtacaccagtcagacgtagattttgtcttttcacatagtcccatatttcttggaggctttgttcatttctttttactcttttttctctaaacttctcttcttgctttatttcattaatttgatcttgaatcactgataccctttcttccacttgatcaaattggctattgaagcttatgcatgtgtcacgtagttctcatgccatggttttcagctctatcaggtcatttaaggtcttctctacactgtttattctagttagccatttgtctaatcttttttcaaggtttttagcttccttgcgatgggttcaaacattctcctttagctcagagaagtttgtcattaccgaccttctgaagtctacttctgtcagcttgtcaaagtcattctttgtccagctttgttctgctgctggcgaggagctgcgaccctttggaggagaagagactttctggtgtttagaattttcagcttttctgctctggtttctccccatctttgtggtttttttctacctttggtctttgatgttagtgacctacagatggggttttggtgtggatgtccttttagttgatgttgatgctattcctttctgtttgttagttttccttctaacaggtccctcagctgcaggtctgttggagtttgctggaggtccactctagaccctgtttccctgggtatcaccagcagaggctgcagaacagtaaatattgcagaacagcaaatatggctgcctgatccttcttctggaagcttcgtcccagaggggcacctgcctgtatgaggtgtcagtcgacccctactgggaggtgtctcccagttaggctacacgggggtcaggcacccacttgaggaggcagtctgtccattctcagagctcaaaccctgtgctggcagaaccactgctctcttcagagctgtcagacagggacgtttaagtctgcagaagtttctgctgccttttgttcagctatgccctgcccccagaggtggagactacagaggcagcaggccttggtgagctgaggtgggctccacccagttcaagcttccctggctgctttgtttacctactcaagcctcagcaatggcggacacccctccccctgccaggcttgctgcctcgcagttctatctcagactaacagtgagcaaggttctgtgggtgtggcatataatctcctggtgtgccgtttgcaagaccattggaaaagtgcactatttgggtgggagtgtcccatttttccaggtacagtctgttacgcttcccctggctaggaaaaggaaatcccctgaccccttgtgcttcctggatgaggcaatgccccgccctgctttgagttgccctctgtgggctgcacccactgtccaaccagtcctggtgagatgaaccaggtacctcagttggaaatgcagaaatcacccatcttctgcatctatcacgctgggagctgcagactggagctgttcctattcggccatcttggaatggaatcttttttttttttttttttttttagatggagtcttgctctgtcgccaggctggagggctagagtgcagtggtgtgatctcagctcactgtaacctccgtgtcctaggttcaagcaattctcctgcctcagcctcctgaatagcttggattacaggcaagcaccactacacctggctaatttttgtatttttagtagagatggggtttcaccatgttggtcagactggtcttgaactcctgacctcatgatccgcctgcctcagcctcccaaagtgctgggattataggcataagccattgcacctggccgaggttttcttttatctgtttggcctgggaaaaattgttgagagtaggccatataggggcatacgattgtccccagcaaagtggtgctatttgcaacctcctctccccagctcacactctgcattccccaggtgggactgcctcactcctctggtgygtgggttgctttgagctgatactattttttcttcattcaatggagtggccttctatctctcatattctaatattgacagtttttcacattttaacatttctgaaacaggttgtggcttaaaattgatggcatcttatagtttatgacatatggcagctgactcagactcacctgtcagccattctcaggccaggtttgtgtcctgatgttgcccctggggtgaaattaaagcttctttttatttttagtttttaagtttgtgtgtgtgtgtgtgtgttttgagatggagtcttattctgtcacccaggctggagtgcagtggcgcagtgatctcggctcactgcaacctccgcatcctgggttcaagcaattttcctgcatcagcctccacagcaggtaggattacagatgtgccaccatcacacctggctaatttttgtattttcagtagagatggggtttcaccatgtttgccaggatggtctcgaactcctgacctcaagtgattcacccacctcagcctcccaaagtgctgggattataggcatgagccaccttgctggtgcaattaaagcttctt

SEQ ID NO: 77 - DNA, 52 bp, Homo sapiens
gtgagggggagagcaggattcacggcrtggactgtggagctcagccccttcc

SEQ ID NO: 78 - DNA, 401 bp, Homo sapiens
ctccagcctaggcaacagagtgatacccaatcttgaaaacaaacaaaaaaccagaacacatttttttgtaccagcaaactattacctggaagccgtcatgtggtgtagcatttgcaaagcgctctaccgataaggacgttctggtttagccataccctacgcttttacattcctgtgagggggagagcaggattcacggcrtggactgtggagctcagccccttcctggctgggtgatgactgaccatggtcacagcccctccaccaccttacgttcccttccctggaagcagactttgaatgtgacggcttcatttgggaggtgcaggaaatggtgttgggagtgaggaaagtaaggcagccagtaaagggtgtgttattaaaactgcagcagtgtgcaa

SEQ ID NO: 79 - DNA, 52 bp, Homo sapiens
gaactctgaagattaaatcaaggattkgaagatagggaacttatcctgaatt

SEQ ID NO: 80 - DNA, 888 bp, Homo sapiens
ttgattacagagatttattagcacagcgggggaatatgtaggggccatctcagaactgtgttgacaaaggcacaaactagcaagaatacagagagacttcttggaagggagggatggtgtcagggagtaaggacagagaggggaatgaggtgggtgacagagtggcaacatgtttttgttggacataggtcattacatagtatggtgggaagaataacgaagacttcacctcccaatccctgggatctgtgaatgtgtcaccttacatggcaaaaggaactctgaagattaaatcaaggattkgaagatagggaacttatcctgaattgtctgggtgtgtgccatgtaatcataaggctccttattggggaaagatggaggcaggaccaggagaaagggagatgtgatgatggaagcaggggtgtaagtgatgcctttaatctggctttgaagatagaaggggccacgagccaaagaatgtgggcagcctccagacactggaaagggtaagcaagtgaattctctcatagagcctcctctaaggaacacagtctgttgacacattgagtttagcccaggctgactcattttggacttctgacctgcagaactgtgagataataaacttgtgttgttttaagccaataagcttatggtaaatttgttacagcagcaatagtaaactaattcccacaggaatccagggggcttccttcttggtcctcctatgtgtctggcacaaatgagaggcccttgttgtagctgacaaagagggccctgttactcaaaccagcagtgctgacacagcagaaactgcttcagcactgaagcgaaggagcgtgggcgggaggagcccccgactgtgtggagagggctggggaggcaggt

SEQ ID NO: 81 - DNA, 52 bp, Homo sapiens
gacagaggtattgaatggtccgatgtytgtgtctcactgtgctgtgctggat

SEQ ID NO: 82 - DNA, 401 bp, Homo sapiens
gaccccaccaagcccacaccttagtcttgtctgattgctaagagggggctctctaggtccagcatagagtccaagtgacctcacaggaaggtttgtggctggaggaaggggaggtcaagtgatgattggtcagtcagaagatcctagtggtggctgggccaggggacttctaaggacagaggtattgaatggtccgatgtytgtgtctcactgtgctgtgctggatgtcactttccacacctgctttgaaagtctgcagcgggcaagaatcacaacgtccagtcctacaggtcaggccagtgtggatgagagagtgtgctcacaggaagcagagggtgacacacagcagggcagtcccaggaagcagagacttggagttaaagataactcagaggaacaag

SEQ ID NO: 83 - DNA, 52 bp, Homo sapiens
tgagagtcacagaattttacatatgascattttttaataaatccataataca

SEQ ID NO: 84 - DNA, 201 bp, Homo sapiens
agagaaaactagagggcttatggaatgatgctaaaaacttagtaacacacatttttaaaacgcagaatcctaattgagagtcacagaattttacatatgascattttttaataaatccataatacatagaaaatacctgaagtttacttgtggaaggattgcagcttgtgagttattctgaagtgatggatggatgataat

SEQ ID NO: 85 - DNA, 52 bp, Homo sapiens
ctttgtcaaagtgtgaggattctcccrctccaatgaaagtaacaccaatagt

SEQ ID NO: 86 - DNA, 501 bp, Homo sapiens
cagatttctacaatgttaatgtttatcatactttctgcatatattacttattagaaatataatttaaaataaataaatgttttcatagtctcactttccctgggtcataacaattacaaaaaaagtatttctttagcttcatgcctaagcactatgaagatactccagctcacaaagtcaaaggctgaaagaactgatggctatgcctaactgtcatattacagctttgtcaaagtgtgaggattctcccrctccaatgaaagtaacaccaatagtgctcctgataccttttaagaaagagatgttataccaggcttctggcggagggccgccaggcttgaattcttggataggctgtgtcactaccaaattgtactgagtagcacctctttctgcttaaccttgtgtagctttctgaacctaatttttgtgcctcacccgtatttccccttaggcttgccaagttcctcaagagacaatttggtttctttttatccttca

SEQ ID NO: 87 - DNA, 52 bp, Homo sapiens
acacctccctccgtgaagccaaaaccsgggtgggaggagaggacagatatca

SEQ ID NO: 88 - DNA, 672 bp, Homo sapiens
gggcctctgcccttctggccagtgtgggagagggtggacatgagtgtacacaagggaactgatggaacaatgggagagacggagggaaggggaggggctctaatccgtcaaggagatacctgcctttttgtggatagggtggggccgggcggcaggagccagcatctggtaacaatatccacaaagatgacagcacctgctctgttccgggctgttctaggggctctaccatatgctcgtctaatcttcacagcaacctcatgactagacaggattacaatctacattttggagaggggacaccaaggcctcgaggggttagaaacctgcccaaggtcacacaactgggcgggaggtgctcagccccacagacagcttcagagctggagcacttgagcccacacctccctccgtgaagccaaaaccsgggtgggaggagaggacagatatcagggaagaacctgcagatgcccagagcttgtgccaatgagccttgcatttgacagattcgagggagtgggttctagaaagcaccactcaacagctcacctagtgagcacctactatgtgccacgggcttgaggcagcagccggcggggaggagtccaggcttgccagcccaactccatgcaccttccagtgaccacactgtgcccagaggggtgggatgta

SEQ ID NO: 89 - DNA, 52 bp, Homo sapiens
tgtgtgggtcacaagagttaaagctcyttggaaattttatggttaagccata

SEQ ID NO: 90 - DNA, 601 bp, Homo sapiens
tactttacaggtgataatacactaaaggaagaacaagctcatctgggagagagaacccaggactagaccttggaaactgtagttctaaacccaaatcttaccccttctggctgtggcccttcagatgggtcacttaaactatctgggcttttgtggcttcatatgtagaatgagagtgttatattaaagagatgaccaagattttttccagctttattttttatttttttaaagaggcttgtgaggagctatttttcagctttaaattgtagtttgtgtgggtcacaagagttaaagctcyttggaaattttatggttaagccatacagtaatgacttagcttcattacataaccacttcatttgtggttagttgctcaattctggtgccatgatcttcagacttcacaaatagatttgtgcaaagcattgtgtaagcatggcatatatagtttaaagaattccttgttgcggaaactccaggttttgttattcgtttgaagggtatgtgtgtttcctcctcctttacttcctccacccccaaatttaggacagggatactagaggcagggaagttttagaaagcacttaaacttttaatg

SEQ ID NO: 91 - DNA, 52 bp, Homo sapiens
ttattatttgacagcttatcaggggcwttcctcctttggttatctcctctct

SEQ ID NO: 92 - DNA, 714 bp, Homo sapiens
tgtaatattattcagctgctctccattgcacacaggaaactagaacatcactaaataaaaatttcctcttattttcaccacccaataaaactcaaaccaaaatttacagccggttcttcattttcccttctttggttgcattggaaaaagctccaatctcatcaaaggccctgatgggctttatttcttattatttgacagcttatcaggggcwttcctcctttggttatctcctctctcactcatcagtgtttctttcttctctggatctttcttatcaccattctaacatggtttcattcctcccataattcacacaagaatgtttttatatatacattaccctgatccctcatctccttccagctgccacccatttccctacttctttttatgggcaactgtctcaaaaaattatgtttgcctcctctctaattccgcacagttcactttctcttcaacctccaccccaatttggcttacgcctccaccacgtctgagattatttttccctgatcaccaatgccttcaaatattgccaaatctaatagattcatctctgtcctcaacctaattaagctctcaatgatttgtcaccattgattactctatctttttgtaatatctcatataatactgtaaggaaagtgaaaaaaaaagaatggttatatgggtactcaaagaagtgtagtttctactgaatgagtattgctt

SEQ ID NO: 93 - DNA, 52 bp, Homo sapiens
cacagaattctactccagtttagtttycaatgttttaacatgagacagaaga

SEQ ID NO: 94 - DNA, 944 bp, Homo sapiens
agctggtcatggtagcacacacctgtactaccagctgctcgggaggctgaggcaggagaatcgcttgaacccgggaggcggaggttgcagtgagccgagatcgcaccactacattccagcctggcaacaaagtgagattccatctcaataacaaaataaaacaaaacaaaacaacaacaacaacaaaaactacctatcagacactatgcttattacctgggtgacaaaataatctgtacaccaaactcccatgacatgcaatttacctatgtaacaaacctgcacgtgtagccctgaacttaaaacaaaagttaaagacatgatttaaacaaatcatattatagatactgataaagctagttaagcactatgcatccaataattattacactaaggcaaaaatacattgatttaaagtctttcttattgactaaataaataacatgactttcttattgcaaattatacttcaatcacagaattctactccagtttagtttycaatgttttaacatgagacagaagagacaaggaatttacgataagatacacttgaatttaaggcttggctctatcagtaaatctcatgtgagatggggcaagtttattgaaccctttctgtctcattttcttttgggtaacattgggatgctgtgaatattaagttggaataatcatatctaacttgtctggtgcatgttaaaagtgccacaaaagttagttctcttttctaattttttgtggcatttattggataccagactagtggcatttattggaaactacttttgaagtatatacatacatacatatatatatatatatatatatatatatatatatatatatatatatatatatatatcttttctctttcctgcattcatttgtaaaagccttgctaattatttttcaattatactcttacaagtcaataatagaaa

SEQ ID NO: 95 - DNA, 52 bp, Homo sapiens
ccaatttttttactgtccgttaaacayggcaggcactgctatcttgtctgtt

SEQ ID NO: 96 - DNA, 801 bp, Homo sapiens
aattccccaagcctctaaatcttactcccagaagtgaggaccattagagcttggtatatatctttccagaactctcctatgtcagtatacccccacacctgactcacacgcacttttttgaaacaagttatttgacagtttctgactctagctgctcaaccttctaatcaagtggttctcaaagtgtgatccttagaccagcagcatcagcatcatctgggaacttgttagaactgcaaattctcggattccactccagaactaatgaatcagaaactctgaaggtcgagcccaggaatatgggttttaataagctctcaaggtaatgctggtgcacactaaagattgagaaccactgctctaagccagccaagccaatttttttactgtccgttaaacayggcaggcactgctatcttgtctgttttatcagccccgattcaaaatgttttcctgtccttttgattaattctgaccctatccacctgtcaaagcccagaaagtcaaaacccaaagtggtctcaccaccacatctcatactgatccttcttgccccctttccccagctcccatgatacagctagtccccctaccatataatttaacaattatatatacgctgacttcaacattagagaacagtgctttactcttccccaacagaccataacctccttgaagacatgaatcatgtttttgtatgtcttctcccacccctgttctaagcacataaatagaatcataatagagttgctagttaatgtctttaaggatgaattcactgagaagaaatactgataa

SEQ ID NO: 97 - DNA, 52 bp, Homo sapiens
tccgggggacgccccactccaaccccrgaggggctctgccacctgcagcccg

SEQ ID NO: 98 - DNA, 401 bp, Homo sapiens
agctgcagccgacacccaagggcctacgtgatggggcgtgatggggcgcgctgggacgcccaggacggggaaaggaaggaaaccccagaagccgaattgggctctgaggaccctgggtccctcccacgctgactgcaccgggccagcccctcacatagtccaagtgactgaggatccgggggacgccccactccaaccccrgaggggctctgccacctgcagcccggctctgacaggccctggggcttcggcaaggcctgtgactgccccgcacctcctcaggggtgagccggacgaagccagcaggccggggccctctcagatgcacacaggaccccccaaacctgggaggagacgaggctggactcagacccaccaagtggagcacaccagggccctct

SEQ ID NO: 99 - DNA, 52 bp, Homo sapiens
tctggccaaaaaaaaaaaaaagggagmaaatcaaaacaaaatgaaacccact

SEQ ID NO: 100 - DNA, 701 bp, Homo sapiens
ttaattcttgtagaatctatacgtgtatagaatctcataagttcttgagtttaatgctattctattcctatcaaattaactttagtaaaattagtataaaccaattggttccatttgtaggtaactacaataaaccttcgttttggtatcctaatgtcatcaaaaagagaatgctctggccaaaaaaaaaaaaaagggagmaaatcaaaacaaaatgaaacccactgtaaacaaaatcaattggcaaatgacaagcacttgggtaattggcagaacctaaactagtaaatcactttgaaggatggtctgtcagtatacagtaagttacacatgcatttacatttgacccaccaagtgcatttctaaatatataccttgacagtttgcctccaatattatgaaaatatatattgacatgtttatttacaaaaaaaatccttcaaattataaaatattgaaatttctttaaattatactttaagttctagggtacatgtgcacaacgtgcaggtttgttgcatatgtatacatgtgccatgttggtgtgctgcacccattaactcatcatttacattaggtatatctcctaattctattcctccctgctccccccaccccacgacaggccctggtgtgtgatgctacccatcctgtgaccatgtgttctcattgttcaattcccacctatgagtgagaacatg